(19) United States
(12) Patent Application Publication
(10) Pub. No.: US 2011/0212933 A1
(43) Pub. Date: Sep. 1, 2011

(54) Antiseptic Compositions for the Treatment of Infections

(76) Inventor: Allan Shapiro, Ladner (CA)

(21) Appl. No.: 12/933,358

(22) PCT Filed: Mar. 20, 2008
(86) PCT No.: PCT/CA08/00536

§ 371 Date: May 18, 2011

(51) Int. Cl. A61K 31/573; A61K 31/045; A61K 31/05; A61K 31/14; A61K 31/221; A61K 31/085; A61P 31/00; A61P 29/00

(52) U.S. Cl. 514/171; 514/724; 514/736; 514/643; 514/546; 514/718

(57) ABSTRACT

The invention provides antiseptic compositions that comprise a wide spectrum antiseptic component (WSAC) and optionally one or more other active ingredients. The WSAC comprises an alcohol, one or more antimicrobial phenol compounds, one or more antimicrobial agents, a solvent and optionally, one or more non-ionic detergents. Additional components such as additional active agents, and thickeners can also be optionally added. The antiseptic compositions have a wide-spectrum of antiseptic activity against microorganisms and exhibit minimal toxicity and minimal irritation and can be used to treat and/or prevent dermal, mucosal, cuticle (such as nail or hoof) or genital infections in a patient.

### FIELD OF THE INVENTION

The invention relates to the field of compositions having antiseptic activity, and in particular to such compositions having a wide-spectrum of antiseptic activity and their use for the treatment and/or prevention of dermal, mucosal, cuticle and genital infections.

### BACKGROUND OF THE INVENTION

Anti-inflammatory ointments, creams, lotions, gels and liquids which contain steroids, such as hydrocortisone or betamethasone, are available for the treatment of dermal, mucosal, cuticle and genital infections. Antibiotic ointments, creams, lotions, gels and liquids are also available. Often, to deal with an inflamed dermal, mucosal, cuticle or genital infection, such as a rash, insect bite or athlete's foot, a physician will prescribe an antibiotic preparation and a separate anti-inflammatory preparation and advise the patient to apply both to the affected area. Some dermal, mucosal, cuticle and genital infections are caused by bacteria, viruses, fungi or protozoans or from combinations of these micro-organisms and, in such cases an antibiotic treatment alone will not be completely effective. In contrast, an antiseptic treatment will kill all classes of micro-organisms because an antiseptic has a much broader spectrum than an individual antibiotic or combinations of antibiotics.

Several products are commercially available that offer either antibiotic or anti-inflammatory properties, or both. However, commercially available products that have antibiotic activity do not provide a broad spectrum antiseptic activity. Furthermore, products containing certain types of antibiotics, when used for prolonged periods of time, can result in the development of superinfections by microorganisms that are resistant to the antibiotics. Examples of commercially available products offering only antibiotic effects include Polysporin® and Neosporin®. Examples of commercially available products offering antibiotic and anti-inflammatory effects include Fucidin® H topicals such as Fucidin® H Ointment and Fucidin® H Cream (having sodium fusidate and hydrocortisone acetate as active ingredients), ratio-Triacomb (having triamcinolone acetonide, neomycin, nystatin, and a gramicidin compound as active ingredients), and Lotriderm cream (having clotrimazole and betamethasone as active ingredients). Although some of these products may be considered wide spectrum antibiotics and may be effective against a number of different bacteria or a class of bacteria, such as gram-negative or gram-positive bacteria, these products do not exhibit antiseptic activity against a wide spectrum of microorganisms, such as for example, against bacteria and fungi or bacteria and viruses.

U.S. Pat. No. 7,338,927 describes a wide spectrum disinfectant including as components an alcohol, O-phenylphenol (OPP), chlorhexidine gluconate, nonoxynol-9, benzalkonium chloride, and deionised, double-distilled water, and methods of preparing same. The wide spectrum disinfectant is described as a safe and effective disinfectant that can be used to disinfect surfaces.

United States Patent Publication No. 2007/0036831 describes nanoemulsion compositions having anti-inflammatory activity with low toxicity that demonstrate broad spectrum inactivation of microorganisms or prevention of diseases. The nanoemulsions contain an aqueous phase, an oil phase comprising an oil and an organic solvent, at least one anti-inflammatory agent, and one or more surfactants, and can be used in the prevention or treatment of a number of dermal, mucosal, cuticle or genital infections. The nanoemulsions are described as having sporicidal activity. The application also provides data indicating that small particle size nanoemulsions are more stable and more effective in inactivating bacteria than standard emulsions. The preparation of nanoemulsion compositions is not necessarily as straightforward as the preparation of solutions, and generally requires specialized equipment. Furthermore, nanoemulsions and emulsions are generally not as stable as solutions over long periods of time.

U.S. Pat. No. 4,604,384 describes a composition for use for the treatment of burns, cuts, wounds, abrasions and the like, which comprises a pharmaceutically acceptable glycol, preferably propylene glycol, and a cellulose derivative, which is heat sterilizable, preferably hydroxyethyl cellulose. The composition optionally contains an antiseptic, an antibiotic and/or a topical corticosteroid.

U.S. Patent Publication No. 2006/0051385 describes antimicrobial compositions, especially those useful when applied topically, particularly to mucosal tissues (i.e., mucous membranes), including a cationic antiseptic such as biguanides and bisbiguanides such as chlorhexidine and its various salts; polymeric quaternary ammonium compounds such as polyhexamethylenebiguanide; silver and various silver complexes; small molecule quaternary ammonium compounds such as benzalkonium chloride and alkyl substituted derivatives; di-long chain alkyl (C8-C18) quaternary ammonium compounds; cetylpyridinium halides and their derivatives; benzethonium chloride and its alkyl substituted derivatives; and octenidine. The compositions can also include an enhancer component, a surfactant, a hydrophobic component, and/or a hydrophilic component. These compositions are described as being useful in the treatment and/or prevention of conditions that are caused, or aggravated by, microorganisms (including viruses).

U.S. Patent Publication No. 2007/0248565 describes a stable preparation of a combination drug, comprising an anti-inflammatory agent and an anti-infective agent. The anti-inflammatory agent is a corticosteroid, and the anti-infective agent is a derivative of quinolone, amino-glycoside or their pharmaceutically acceptable salts. The combination drug essentially comprises i) an anti-inflammatory agent which is a corticosteroid, ii) an anti-infective agent selected from the group comprising derivatives of quinolone, aminoglycoside and their pharmaceutically acceptable salts; iii) a complexation enhancing polymer; iv) a solubiliser exhibiting an inclusion phenomenon, along with pharmaceutically acceptable excipients with a suitable carrier system. Benzalkonium chloride can be included in the preparation as preservative.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide an antiseptic composition for the treatment of infections. In accordance with an aspect of the present invention, there is provided a use of an antiseptic composition comprising a wide spectrum antiseptic component, said wide spectrum antiseptic component comprising an alcohol, one or more antimicrobial phenol compounds, one or more antimicrobial agents, a solvent, and optionally one or more non-ionic detergents, for the treatment of a dermal, mucosal, cuticle or genital infection.

In accordance with another aspect of the invention, there is provided a use of a wide spectrum antiseptic component comprising an alcohol, one or more antimicrobial phenol compounds, one or more antimicrobial agents, a solvent, and optionally one or more non-ionic detergents, in the preparation of an antiseptic composition for the treatment of a dermal, mucosal, cuticle or genital infection.

In accordance with another aspect of the invention, there is provided an antiseptic composition comprising a wide spectrum antiseptic component and optionally one or more of an anti-inflammatory agent, an analgesic, or an anaesthetic, said wide spectrum antiseptic component comprising an alcohol, one or more anti-microbial phenol compounds, one or more anti-microbial agents, a solvent, and optionally one or more non-ionic detergents, with the proviso that when one or more of the anti-inflammatory agent, analgesic, or anaesthetic is absent, the one or more non-ionic detergents are also absent, and when the wide spectrum antiseptic component comprises one or more non-ionic detergents, the antiseptic composition comprises one or more of an anti-inflammatory agent, an analgesic, or an anaesthetic.

In accordance with another aspect of the invention, there is provided a kit comprising the antiseptic composition according to any one of claims 44-78, and instructions for use.

In accordance with another aspect of the invention, there is provided a method of treating a dermal, fungal, cuticle or genital infection in a subject, comprising administering to said subject an effective amount of an antiseptic composition comprising a wide spectrum antiseptic component, said wide spectrum antiseptic component comprising an alcohol, one or more antimicrobial phenol compounds, one or more antimicrobial agents, a solvent, and optionally one or more non-ionic detergents.

### DETAILED DESCRIPTION OF THE INVENTION

The invention provides antiseptic compositions that comprise a wide spectrum antiseptic component (WSAC) and optionally one or more other active agents. The WSAC comprises an alcohol, one or more antimicrobial phenol compounds, one or more antimicrobial agents, a solvent and optionally, one or more non-ionic detergents. The WSAC is prepared as a solution, however, additional components such as thickeners, can optionally be added to this solution in order provide the final antiseptic composition. Accordingly, the antiseptic composition of the invention can be, for example, a liquid, semi-liquid, gel, cream, lotion, foam or the like.

The antiseptic compositions have a wide-spectrum of antiseptic activity against microorganisms. For example in one embodiment, the antiseptic compositions are able to kill bacteria, fungi, viruses and protozoans. The antiseptic compositions exhibit minimal toxicity and minimal irritation. As such the antiseptic compositions can be used to treat and/or prevent dermal, mucosal, cuticle (such as nail or hoof) or genital infections in a patient. Accordingly, methods of using these compositions for the treatment of dermal, mucosal, cuticle or genital infections are also provided.

It is further contemplated that one or more other active agents may optionally be included in the antiseptic composition, for example anti-inflammatory agents to provide a composition with antiseptic and anti-inflammatory activity. Such antiseptic compositions can be used to treat or prevent dermal, mucosal, cuticle, or genital infections that may or may not be accompanied by inflammation.

As indicated above, U.S. Patent Publication No. 2005/0282727 describes a wide spectrum disinfectant including as components an alcohol, O-phenylphenol (OPP), chlorhexidine gluconate, nonoxynol-9, benzalkonium chloride, and deionised, double-distilled water, and methods of preparing same. The wide spectrum disinfectant is safe and effective for disinfection of surfaces, and for preventing establishment of infections in other contexts such as preventing or decreasing the transmission of sexually related diseases.

As described herein, this and related disinfectants have now been found to be surprisingly effective in treating established infections in vivo, and as such, are suitable for inclusion in antiseptic compositions for use to treat such infections in a subject. As also described herein, in various embodiments of the invention, antiseptic compositions comprising diluted versions of these disinfectants as the WSAC unexpectedly retain their efficacy in treating these infections.

In addition, in various embodiments, the antiseptic compositions exhibit one or more useful properties. For example, in one embodiment of the invention, the antiseptic compositions exhibit good stability and thus have a long shelf life. In one embodiment, the antiseptic composition according to the invention is stable when stored for periods of time up to 10 years, at room temperature.

As noted above, the antiseptic compositions exhibit minimal irritation. Thus, in one embodiment, use of the antiseptic composition according to the invention can result in greater patient compliance, in view of minimal irritation to skin that is exhibited by the antiseptic composition.

In one embodiment, the antiseptic composition according to the invention is easily prepared using standard equipment and reagents, i.e. no specialized equipment or reagents are required.

The antiseptic composition according to the invention can be used to treat a variety of established microbial infections. In one embodiment, for example, the antiseptic composition according to the invention can be used to treat one or more of athlete's foot, eczema, ringworm, jock itch, hand and palm infections, impetigo, acne, shingles, seborrheic dermatitis, warts (human papilloma virus), Staphylococcus infections, Tinea versicolor rashes or insect bites. In one embodiment, the antiseptic compositions according to the invention can be used to treat one or more of eczema, Tinea versicolor rashes, Staphylococcus infections, or athlete's foot. In one embodiment, the antiseptic compositions according to the invention can be used to treat one or more of eczema, Tinea versicolor rashes, or athlete's foot.

In one embodiment, the antiseptic composition according to the invention can be used to treat one or more of the preceding conditions wherein the preceding condition is accompanied by inflammation.

### DEFINITIONS

Unless defined otherwise, all technical and scientific terms used herein have the same meaning as commonly understood by one of ordinary skill in the art to which this invention belongs.

As used herein, the term “about” refers to approximately a +/−10% variation from the stated value. It is to be understood that such a variation is always included in any given value provided herein, whether or not it is specifically referred to.

The term “wide spectrum of antiseptic activity” refers to the ability of a composition to kill or inhibit the growth of several types of microorganisms. In one embodiment, a composition having a wide spectrum of antiseptic activity is able to kill, inhibit, or prevent the growth of bacteria, fungi, viruses and protozoans. In another embodiment, a composition having a wide spectrum of antiseptic activity is able to kill, inhibit or prevent the growth of bacteria, fungi, and viruses.

The term “weight percent” or “% (w/w)” refers to a percentage of a component in a solution that is calculated on the basis of weight for the component and the solvent. For example, a 1% (w/w) solution of a component would have 1 g of the component dissolved in a 100 g of solvent. The term “volume percent” or “% (v/v)” refers to a percentage of a component in a solution that is calculated on the basis of volume for the component and the solvent. For example, a 1% (v/v) solution of a component would have 1 ml of the component dissolved in a 100 ml of solvent. The term “weight/volume percent” or “% (w/v)” refers to a percentage of a component in a solution that is calculated on the basis of weight for the component and on the basis of volume for the solvent. For example, a 1% (w/v) solution of a component would have 1 g of the component dissolved in a 100 ml of solvent.

The term “anti-inflammatory agent,” as used herein refers to an agent which is able to reduce inflammation in a subject. Anti-inflammatory agents can be steroidal or non-steroidal, as is known in the art.

The term “antiseptic,” as used herein refers to the property of attenuating an established infection, sepsis, or putrefaction on living tissue or skin. In one embodiment, antiseptic refers to the property of being able to kill a broad spectrum of microorganisms, for example one or more of bacteria, fungi, viruses, or protozoans.

The term “antimicrobial agent” refers to a substance that kills, inhibits, or prevents the growth of microbes such as bacteria, fungi, and viruses.

The term “solution,” as used herein refers to a substantially homogeneous mixture composed of two or more substances. In such a mixture, one or more solute is dissolved in one or more liquids, referred to as solvents.

The term “emulsion,” as used herein refers to a mixture of two or immiscible (unblendable) substances in which at least one substance (the dispersed phase) is dispersed in at least one other (the continuous phase). The term “nanoemulsion,” as used herein refers to emulsions in which the sizes of the particles in the dispersed phase are defined as less than 1000 nanometers.

The terms “therapy” and “treatment,” as used interchangeably herein, refer to an intervention performed with the intention of improving a subject's status. The improvement can be subjective or objective and is related to ameliorating the symptoms associated with, preventing the development of, or altering the pathology of a disease or disorder being treated. Thus, the terms therapy and treatment are used in the broadest sense, and include one or more of prevention (prophylaxis), moderation, reduction, and curing of a disease or disorder at various stages. Preventing deterioration of a subject's status is also encompassed by the term. Subjects in need of therapy/treatment thus include those already having the disease or disorder as well as those prone to, or at risk of developing, the disease or disorder and those in whom the disease or disorder is to be prevented.

The term “ameliorate” includes the arrest, prevention, decrease, or improvement in one or more the symptoms, signs, and features of the disease or disorder being treated, both temporary and long-term.

The term “subject” or “patient” as used herein refers to an animal in need of treatment.

The term “animal,” as used herein, refers to both human and non-human animals, including, but not limited to, mammals, birds and fish.

The term “inhibit,” as used herein, means to prevent, decrease, slow-down or arrest. In one embodiment, a composition of the invention is considered to inhibit a process or reaction when the amount or rate of the process or reaction that takes place in the presence of the composition is decreased by at least about 10% when compared to the amount or rate in the absence of the composition. In another embodiment, a composition is considered to inhibit a process or reaction when the amount or rate of the process or reaction that takes place in the presence of the composition is decreased by at least about 20% when compared to the amount or rate in the absence of the composition. In other embodiments, a composition is considered to inhibit a process or reaction when the amount or rate of the process or reaction that takes place in the presence of the composition is decreased by at least about 25%, about 30%, about 40%, about 50%, about 60%, about 70%, about 75% or about 80% when compared to the amount or rate in the absence of the composition. In one embodiment, a process or reaction is growth of a microorganism.

The term “active agent” as used herein refers to an agent that provides a therapeutic benefit to a subject. For example, an active agent includes anti-inflammatory compounds, analgesics, and anesthetics as non-limiting examples.

### Antiseptic Compositions

As noted above, the antiseptic compositions according to the invention comprise a wide spectrum antiseptic component (WSAC). The compositions may optionally further comprise one or more other active agents and/or one or more formulating agents. Thus in its simplest embodiment, the invention provides for an antiseptic composition consisting of a WSAC. In other embodiments, the invention provides for antiseptic compositions comprising a WSAC and one or more other components such as active agents and/or formulating agents.

In one embodiment, the antiseptic composition comprises a wide spectrum antiseptic component and optionally one or more of an anti-inflammatory agent, an analgesic, or an anaesthetic, said wide spectrum antiseptic component comprising an alcohol, one or more anti-microbial phenol compounds, one or more anti-microbial agents, a solvent, and optionally one or more non-ionic detergents, with the proviso that when one or more of the anti-inflammatory agent, analgesic, or anaesthetic is absent, the one or more non-ionic detergents are also absent, and when the wide spectrum antiseptic component comprises one or more non-ionic detergents, the antiseptic composition comprises one or more of an anti-inflammatory agent, an analgesic, or an anaesthetic.

The WSAC included in the antiseptic compositions according to the invention comprises an alcohol, one or more antimicrobial phenol compounds, one or more antimicrobial agents, one or more non-ionic detergents, and double-distilled, deionized water. Thus in one embodiment, the WSAC comprises an alcohol, one antimicrobial phenol compound, one antimicrobial agent, one non-ionic detergent, and deionised, double-distilled water. In one embodiment, the WSAC comprises an alcohol, one antimicrobial phenol compound, two antimicrobial agents, one non-ionic detergent, and deionised, double-distilled water. In one embodiment, the WSAC comprises an alcohol, one antimicrobial phenol compound, two antimicrobial agents, two non-ionic detergents, and deionised, double-distilled water. In one embodiment, the WSAC comprises an alcohol, one antimicrobial phenol compound, one antimicrobial agent, two non-ionic detergents, and deionised, double-distilled water.

In one embodiment, the WSAC comprises an alcohol, two antimicrobial phenol compounds, one antimicrobial agent, one non-ionic detergent, and deionised, double-distilled water. In one embodiment, the WSAC comprises an alcohol, two antimicrobial phenol compounds, two antimicrobial agents, one non-ionic detergent, and deionised, double-distilled water. In one embodiment, the WSAC comprises an alcohol, two antimicrobial phenol compounds, two antimicrobial agents, two non-ionic detergents, and deionised, double-distilled water. In one embodiment, the WSAC comprises an alcohol, two antimicrobial phenol compounds, one antimicrobial agent, two non-ionic detergents, and deionised, double-distilled water.

As indicated above, the antiseptic compositions can include an anti-inflammatory agent. Thus in one embodiment, the antiseptic composition comprises a WSAC as described above, and an anti-inflammatory agent.

In accordance with the invention, the antiseptic composition has a neutral pH and low ionic strength, and the amounts of the individual components that make up the composition are therefore selected such that the final composition retains a neutral pH and low ionic strength. By neutral pH it is meant a pH between about 5 to about 9. By low ionic strength it is meant an ionic strength between about 0.0001M and 0.3M. Appropriate amounts of the recited components can be readily selected by a worker skilled in the art.

As indicated above, the antiseptic compositions according to the invention are stable over long periods of time at room temperature. In one embodiment, the antiseptic compositions according to the invention are stable for up to 3 years. In one embodiment, the antiseptic compositions according to the invention are stable for up to 5 years. In one embodiment, the antiseptic compositions according to the invention are stable for up to 8 years. In one embodiment, the antiseptic compositions according to the invention are stable for up to 10 years.

In one embodiment, the WSAC comprises at least 50% (w/w) alcohol, appropriate amounts of one or more antimicrobial phenol compounds, one or more antimicrobial agents, and one or more non-ionic detergents to provide a neutral pH, and low ionic strength. In one embodiment, the WSAC comprises at least 50% (w/w) alcohol and from about 0.001% to about 5% of an antimicrobial phenol compound, and appropriate amounts of the other components to provide a neutral pH and low ionic strength. In one embodiment, the WSAC comprises at least 50% (w/w) alcohol, from about 0.001% to about 5% of a quaternary ammonium compound as one or more antimicrobial agents, and appropriate amounts of the other components to provide a neutral pH, and low ionic strength. In one embodiment, the WSAC comprises at least 50% (w/w) alcohol, from about 0.01% to about 10% of a biguanide compound as one or more antimicrobial agents, and appropriate amounts of the other components to provide a neutral pH, and low ionic strength. In one embodiment, the WSAC comprises at least 50% (w/w) alcohol, from about 0.01% to about 10% of a non-ionic detergent, and appropriate amounts of the other components to provide a neutral pH, and low ionic strength. In one embodiment, the WSAC comprises at least 50% (w/w) alcohol, from about 0.01% to about 10% of an anti-inflammatory agent, and appropriate amounts of the other components to provide a neutral pH, and low ionic strength.

In one embodiment, the WSAC comprises the following amounts of its constituent components:
from about 50% to about 95% (w/w) alcohol;
from about 0.001% to about 5% (w/w) of each of one or more antimicrobial phenol compound;
from about 0.001% to about 5% (w/w) of each of one or more antimicrobial agents;
from about 0.02% to about 1% (w/w) of each of one or more non-ionic detergents; and
from about 5% to 50% (w/w) of deionised, double-distilled water;

The preceding ranges refer to amounts of constituent components of the WSAC required to prepare a 1× concentration of the WSAC. As will be described in more detail in relation to the preparation of the WSAC, the WSAC can be prepared as concentrates of up to 100× and diluted prior to use. The WSAC can be included in the antiseptic composition, however, from about 0.01× to about 2× concentration. In one embodiment, the WSAC is included in the antiseptic composition from about 0.01× to about 1× concentration.

As noted above, the antiseptic composition is able to kill or inhibit the growth of microorganisms. In one embodiment the antiseptic composition is able to kill or inhibit the growth of microorganisms including bacteria. Examples of such bacteria include Gram positive and Gram negative aerobic and anaerobic bacteria, including Baccilus, Enterobacter, Aeromonas, Aquaspirillum, Nitrosovibrio, Kurthia, Aeromonas, Cytophaga, Scytonema, Azomonas, Gardnerella, Staphylococci, Enterococci, Streptococci, Haemophilus, Moraxella, Escherichia, Chlamydia, Rickettsiae, Pseudomonas, Serratia, Propionibacterium, Mycobacterium, Salmonella, and Mycoplasma, for example. Non-limiting examples of such bacteria include those that are members of the genera. Exemplary species of these genera include Bacillus megaterium, Enterobacter gergoviae, Aeromonas hydrophila, Aquaspirillum gracile, Nitrosovibrio tenuis, Enterobacter gergoviae, Kurthia gibsonii, Cytophaga agarovorans, Scytonema sp., Enterobacter gergoviae, Bacillus acidocaldarius, Cytophaga succinicans, Aquaspirillum itersonii, Azomonas insignis, Aquaspirillum aquaticum, Gardnerella vaginalis, Staphylococcus epidermis, Staphylococcus aureus, Staphylococcus hominis, Pseudomona fluorsecens, Pseudomonas facilis, Pseudomonas aeruginosa, Serratia marcescens, Propionibacterium acne, Enterococcus faecalis, Streptococcus pneumoniae, Haemophilus. influenza, Escherichia coli, Moraxella catarrhalis, Mycoplasma pneumoniae, and Mycobacterium tuberculosis.

In one embodiment, the antiseptic composition is able to kill or inhibit the growth of microorganisms including fungi. Non-limiting examples of such fungi include those that are members of the genera Candida, Trichophyton, Cryptococcus, Microsporum, Arthroderma, Epdermophyton, and Aspergillus. Exemplary species falling within these genera include Candida albicans, Trichophyton mentagrophytes, Aspergillus niger, Cryptococcus neoformans, Cryptococcus gatti, Microsporum audouinii, Microsporum canis, Microsporum canis var. distortum, Microsporum cookei, Microsporum equinum, Microsporum ferrugineum, Microsporum fulvum, Microsporum gallinae, Microsporum gypseum, Microsporum nanurn, Microsporum persicolor, Arthroderma gertleri; Arthroderma gloriae; Arthroderma gruby, Epidermophyton floccosum and Aspergillus fumigatus. In one embodiment, the antiseptic composition is able to kill or inhibit the growth of fungi belonging to the genera Candida, Trichophyton, and Aspergillus.

In one embodiment, the antiseptic composition is capable of killing or inhibiting the growth of microorganisms including viruses. Non-limiting examples of viruses include Human papillomavirus (HPV), human immunodeficiency virus-1 (HIV-1), Herpes (such as Herpes Simplex Virus-1, or Herpes zoster), Epstein-Bar virus or Polioviruses (such as Poliovirus-1). Additional non-limiting examples of such viruses include human cytomegalovirus, and varicella zoster virus.

In one embodiment, the antiseptic composition is capable of killing or inhibiting the growth of microorganisms including protozoans. Non-limiting examples of such protozoans include Trichomonas vaginalis, Leishmania major, Leishmania tropica, Leishmania aethiopica, Leishmania mexicana and Leishmania (Viannia) braziliensis.

In one embodiment, the antiseptic composition is capable of killing or inhibiting the growth of bacteria, fungi, viruses and protozoans.

In one embodiment the antiseptic composition is a mixture other than a nanoemulsion. Examples of such mixtures include, but are not limited to, solutions, standard emulsions and colloidal dispersions.

### Alcohols

Alcohols that can be used in the WSACs of the invention include ethanol, methanol, 1-propanol, or combinations of thereof. As is known in the art, there are two forms of ethanol generally available: denatured ethanol, and potable alcohol. Both denatured and potable ethanols can be used in the preparation of the WSACs noted above. In one embodiment, the alcohol included in the WSAC is a potable ethanol. In another embodiment, the alcohol of the WSAC is a denatured ethanol. Denatured ethanol contains additives for the purpose of preventing or reducing abuse or consumption of the alcohol. In one embodiment of the invention, the alcohol of the WSAC is an ethanol that includes additives such as for example, isopropanol, or Bitrex™, that do not adversely affect the efficacy of the antiseptic composition, or that are not toxic to the subject being treated with the antiseptic composition. In one embodiment the alcohol included in the WSAC is a specially denatured alcohol (SDAG). Examples of SDAGs include SDAG-3, SDAG-6 and SDAG-9. SDAG-3 contains Bitrex™ at 700 mg per 100 litres of ethanol, SDAG-6 contains Bitrex™ at 1 g per 100 litres of ethanol, and SDAG-9 contains iso-propyl alcohol at 5 litres in 100 litres of ethanol. In one embodiment, the WSAC comprises an SDAG alcohol that is denatured with an additive that does not render the antiseptic composition toxic to a subject, or that otherwise interferes with the antiseptic and/or anti-inflammatory activity of the antiseptic composition.

In one embodiment, the antiseptic composition comprises an alchohol wherein the alcohol is present in an amount from about 50% (w/w) to about 95% (w/w). In one embodiment, the antiseptic composition comprises an alchohol wherein the alcohol is present in an amount from about 60% (w/w) to about 80% (w/w).

### Antimicrobial Phenol Compounds

As indicated above, the WSACs of the invention include one or more microbicidal phenol compounds. Suitable microbicidal phenol compounds are selected on the basis that they exhibit minimal toxicity and irritation, and maintain the non-ionic nature of the antiseptic composition. Microbicidal phenols suitable for inclusion in the WSACs of the invention have the following general formula:

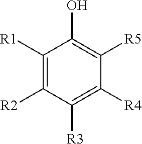

wherein:
R1, R2, R3, R4 and R5 are independently hydrogen, halogen, hydroxy, phenyl, C1-C6 alkyl, C2-C6 alkenyl, C2-C6 alkynyl, C3-C7 cycloalkyl, C3-C7 cycloalkenyl, C1-C6-O-alkyl, C2-C6-O-alkenyl, C2-C6-O-alkynyl or —(CH₂)ₙPh (or C6-C10 aryl), where n is 0-4;
wherein phenyl, cycloalkyl, cycloalkenyl, alkyl, alkenyl and alkynyl are optionally substituted with one or more of halogen, C1-C6 alkyl, C1-C6 alkoxy or hydroxy.

Non-limiting examples of suitable microbicidal phenol compounds include o-phenylphenol (2-oxydiphenyl, 2-phenylphenol, or OPP), benzylphenol, p-chloro-m-cresol, 2,3,4,6-tetrachlorophenol, 2,4-dichlorophenol, monochlorophenylphenol, o-benzyl-p-chlorophenol, 2-cyclopentyl-4-chlorophenol, chlorinated xylenols, resorcinol, thymol (3-hydroxy-p-cymol), eugenol (4-hydroxy-3-methoxy-1-allylbenzene) and carvacrol (2-hydroxy-1-methyl-4-isopropylbenzene). In one embodiment, the WSAC according to the invention includes antimicrobial phenols in the free form of the phenols. In one embodiment, the WSAC according to the invention includes antimicrobial phenols in the salt form of the phenols. These antimicrobial phenol compounds can be prepared by art-known methods or are commercially available from suppliers such as Sigma-Aldrich, TG International Chemical Co. and others.

In one embodiment, the WSAC comprises one or more antimicrobial phenol compounds wherein each antimicrobial phenol compound is present in an amount of from about 0.001% (w/w) to about 5% (w/w). In one embodiment, the WSAC comprises one or more antimicrobial phenol compounds wherein each antimicrobial phenol compound is present in an amount of from about 0.1% (w/w) to about 0.5% (w/w).

### Antimicrobial Agents

The WSAC according to the invention comprises antimicrobial agents. Suitable antimicrobial agents are selected on the basis that they exhibit minimal toxicity and irritation, and maintain the non-ionic nature of the antiseptic composition. Non-limiting examples of suitable antimicrobial agents include quaternary ammonium compounds, anionic and cationic surfactants, and biguanide derivatives. In one embodiment of the invention, the WSAC includes one or more antimicrobial agents. In another embodiment, two or more antimicrobial agents are included in the WSAC. In still another embodiment, three or more antimicrobial agents are included in the WSAC. In yet another embodiment, four or more antimicrobial agents are included in the WSAC.

### Quaternary Ammonium Compounds

Quaternary ammonium compounds are also suitable antimicrobial agents for inclusion in the WSAC. Examples of quaternary ammonium compounds include but are not limited to benzalkonium chloride (BZK), dimethylbenzyldodecylammonium chloride, dimethylbenzyltetradecylammonium chloride, dimethylbenzyl decylammonium bromide, dimethylbenzyloctylammonium chloride and cocosalkyldimethylbenzylammonium chloride, in which the cocosalkyl residue is obtained from the hydrogenated fatty acid mixture of coconut oil. Additional non-limiting examples of quaternary ammonium compounds include cetylpyridinium halides, cetyltrimethylammonium halides, cetyldimethylethylammonium halides, cetyldimethylbenzylammonium halides, cetyltributylphosphonium halides, dodecyltrimethylammonium halides, tetradecyltrimethylammonium halides, alkylbenzyldimethylammonium salts and combinations comprising one or more of the foregoing compounds. Suitable halides in the cationic halogen-containing compounds include chloride, fluoride, bromide and iodide.

### Cationic Surfactants

Cationic surfactants are also antimicrobial agents suitable for inclusion in the WSACs of the invention. Non-limiting examples of suitable cationic surfactants include those based on quaternary ammonium cations such as cetyl trimethylammonium bromide (CTAB, hexadecyl trimethyl ammonium bromide), and other alkyltrimethylammonium salts, cetylpyridinium chloride, polyethoxylated tallow amine (POEA), benzalkonium chloride (BZK), benzethonium chloride (BZT), and chlorhexidine gluconate.

### Biguanide Derivatives

Biguanide derivatives are also suitable antimicrobial agents for inclusion in the WSAC of the invention. Examples of such biguanide compounds are oligohexamethylene biguanide salts having a chain corresponding to the following formula:

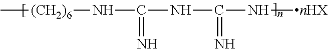

in which HX is a salt-forming acid component and n is a number of at least 2 and preferably of from 4 to 6. Oligomeric biguanides such as these and their production are described in British Patent No. 702,268, in British Patent No. 1,152,243 and in British Patent No. 1 434 040. Examples of salts of these biguanides which are suitable for incorporation in the combinations according to the invention are the corresponding water-soluble mineral acid salts, for example oligohexamethylene biguanide hydrochloride. Bisbiguanides such as these have been repeatedly described in the relevant patent literature, for example, U.S. Pat. Nos. 4,420,484, 2,684,924, 2,990,425, 3,468,898, 4,022,834, 4,053,636, British patent 1,344,042 and U.S. Pat. No. 4,198,392. The following are specific examples of the bisbiguanides that can be included in the WSAC of the present invention: 1,2-bis-(N⁵-p-chlorophenyl-N¹-biguanido)-ethane, 1,2-bis-(N⁵-p-nitrophenyl-N¹-biguanido)-ethane, chlorobenzyl-N¹-biguanido)-ethane, 1,2-bis-(N⁵-p-bromophenyl-N⁵-hexyl-N¹-biguanido)-ethane, 1,2-bis-(N⁵-p-chlorophenyl-N⁵-2-ethylphenyl-N¹-biguanido)-ethane, 1,2-bis-(N⁵-p-chlorophenyl-N¹-ethyl-N-biguanido)-ethane, 1,2-bis-(N⁵-p-methoxyphenyl-N-biguanido)-ethane, 1,2-bis-(N⁵-p-methylphenyl-N¹-biguanido)-ethane, 1,2-bis-(N⁵-3,5-dimethylphenyl-N biguanido)-ethane, 1,2-bis(N⁵-2,6-dichlorophenyl-N′-biguanido)-ethane, 1,2-bis(N⁵-2,6-dimethylphenyl-N¹-biguanido)-ethane, 1,4-bis(N⁵-p-chlorophenyl-N¹-biguanido)-butane, bis-(N⁵-p-chlorophenyl-N-biguanido)-methane and 1,3-bis-(N⁵-p-chlorophenyl-N¹-biguanido)-propane and water-soluble, non-toxic addition salts thereof, particularly gluconates, hydrochlorides and acetates. Another exemplary bisbiguanide is 1,1′-hexamethylene-bis-[5(4-chlorophenyl)-biguanide] in the form of its salts, for example the acetate, hydrochloride or gluconate.

In one embodiment, the WSAC comprises one or more antimicrobial agents wherein each are present in an amount of from about 0.001% (w/w) to about 5% (w/w). In one embodiment, the WSAC comprises one or more antimicrobial agents wherein each are present in an amount of from about 0.005% (w/w) to about 2% (w/w). In one embodiment, the WSAC comprises an antimicrobial agent wherein the antimicrobial agent is present in an amount from 0.1% (w/w) to about 2% (w/w). In one embodiment, the WSAC comprises an antimicrobial agent wherein the antimicrobial agent is present in an amount from 0.005% (w/w) to about 0.5% (w/w).

### Non-Ionic Detergents

Non-ionic detergents suitable for inclusion in the WSAC of the invention include alkyl poly(ethylene oxide) detergents, such as for example nonoxynol-9, nonoxynol-10, and nonoxynol-11, copolymers of poly(ethylene oxide) and poly(propylene oxide) (commercially called Poloxamers or Poloxamines), alkyl polyglucosides such as octyl glucoside and decyl maltoside, fatty alcohols such as cetyl alcohol and oleyl alcohol, or cocamide MEA, cocamide DEA, or cocamide TEA.

In one embodiment the WSAC comprises a non-ionic detergent wherein the non-ionic detergent is present in an amount from about 0.2% (w/w) to about 10% (w/w). In one embodiment the WSAC comprises a non-ionic detergent wherein the non-ionic detergent is present in an amount from about 0.04% (w/w) to about 0.1% (w/w).

### Deionized Double-Distilled Water

The WSACs of the present invention are prepared in a solvent that provides a non-ionic environment in order to solubilize antimicrobial agents such as the phenol compounds. Exemplary, non-limiting examples of such a solvent include water, including deionized water, distilled water, double-distilled water, deionized distilled water, deionized double-distilled water, glycerol, dimethyl sulphoxide, ethylene glycol, diethylene glycol, monoethyl and monobutyl ethers of ethylene glycol and mono methyl, mono ethyl and mono butyl ethers of diethylene glycol. In one embodiment, the WSAC is prepared in water, deionized water, distilled water, double-distilled water, deionized distilled water, or deionized double-distilled water.

### Optional Anti-Inflammatory Agents

The antiseptic compositions of the invention optionally include one or more anti-inflammatory agents such as steroidal and non-steroidal anti-inflammatory compounds. In one embodiment, the antiseptic composition includes one or more steroids. As is known in the art, steroids can be classified as very potent, potent, moderately potent, or mild.

Very potent steroids include, for example, betamethasone dipropionate (Diprolene), clobetasol 17-propionate (Dermovate), halobetasolpropionate (Ultravate), halcinonide (Halog).

Potent steroids include, for example, amcinonide (Cyclocort), betamethasone dipropionate (Diprolene, generics), betamethasone valerate (Betaderm, Belestoderm, Prevex), desoximetasone (Desoxi, Topicort), diflucortolone valerate (Nerisone), fluocinonlone acetonide (Derma, Fluoderm, Synalar), fluocinonide (Lidemol, Lidex, Tyderm, Tiamol, Topsyn), and mometasone furoate.

Moderately potent steroids include, for example, betamethasone valerate (Betnovate), betamethasone valerate (Celestoderm), clobetasone 17-butyrate (Eumovate), desonide (Desocort), hydrocortisone valerate (Westcort, Hydroval), prednicarbate (Dermatop), triamcinolone acetonide (Kenalog, Traiderm).

Mild steroids include, for example, loratodine (Claratin), desonide (Desocort), hydrocortisone (Cortate, Cortoderm), hydrocortisone acetate (Cortef, Hyderm), or a combination thereof.

In one embodiment, the antiseptic composition comprises one or more anti-inflammatory agent that can be very potent, potent, moderately potent or mild. In another embodiment, the antiseptic composition comprises one or more anti-inflammatory agent that is moderate or moderately potent or mild.

In one embodiment, the antiseptic compositions according to the invention comprise an anti-inflammatory agent that is a non-steroidal anti-inflammatory drug. Non-limiting examples of suitable non-steroidal anti-inflammatory drugs include aspirin (Anacin, Ascriptin, Bayer, Bufferin, Ecotrin, Excedrin), choline and magnesium salicylates (CMT, Tricosal, Trilisate), choline salicylate (Arthropan), celecoxib (Celebrex), diclofenac potassium (Cataflam), diclofenac sodium (Voltaren, Voltaren XR), diclofenac sodium with misoprostol (Arthrotec), diflunisal (Dolobid), etodolac (Lodine, Lodine XL), fenoprofen calcium (Nalfon), flurbiprofen (Ansaid), ibuprofen (Advil, Motrin, Motrin IB, Nuprin), indomethacin (Indocin, Indocin SR), ketoprofen (Actron, Orudis, Orudis KT, Oruvail), magnesium salicylate (Arthritab, Bayer Select, Doan's Pills, Magan, Mobidin, Mobogesic), meclofenamate sodium (Meclomen), mefenamic acid (Ponstel), meloxicam (Mobic), nabumetone (Relafen), naproxen (Naprosyn, Naprelan), naproxen sodium (Aleve, Anaprox), oxaprozin (Daypro), piroxicam (Feldene), rofecoxib (Vioxx), salsalate (Amigesic, Anaflex 750, Disalcid, Marthritic, Mono-Gesic, Salflex, Salsitab), sodium salicylate, sulindac (Clinoril), tolmetin sodium (Tolectin), valdecoxib (Bextra), or a combination thereof.

As is known in the art, the type and amount of anti-inflammatory agent included in the antiseptic composition will vary depending on, for example, the potency of the anti-inflammatory agent used, the condition and or subject to be treated, the severity of the condition, and the like.

In general, a potent steroid will be used in an amount less than that of a mild steroid. The antiseptic composition according to the present invention comprises an anti-inflammatory agent in an amount from about 0.01% (w/w) to about 10% (w/w). In one embodiment, the antiseptic composition of the invention comprises an anti-inflammatory agent in an amount from about 0.05% (w/w) to about 5% (w/w). In another embodiment, the antiseptic composition of the invention comprises an anti-inflammatory agent in an amount from about 0.15% (w/w) to about 3% (w/w). In still another embodiment, the antiseptic composition of the invention comprises an anti-inflammatory agent in an amount from about 0.05% (w/w) to about 0.5% (w/w). In another embodiment, the antiseptic composition of the invention comprises an anti-inflammatory agent in an amount from about 0.1% (w/w) to about 0.4% (w/w). In yet another embodiment, the antiseptic composition of the invention comprises an anti-inflammatory agent in an amount from about 1% (w/w) to about 4% (w/w). In yet another embodiment, the antiseptic composition of the invention comprises an anti-inflammatory agent in an amount from about 0.01% (w/w) to about 5% (w/w). In yet another embodiment, the antiseptic composition of the invention comprises an anti-inflammatory agent in an amount from about 0.01% (w/w) to about 2% (w/w).

As is also known in the art, the anti-inflammatory agent to be included in the antiseptic composition will depend on the condition to be treated. For example, hydrocortisone is suitable for treating eczema, Tinea versicolor, and Tinea pedis (athlete's foot), while betamethasone is suitable for treating Tinea versicolor, dermal eczema with secondary Staphylococcus infections, and Tinea pedis.

### Other Additives

In one embodiment, other additives such as analgesics, anaesthetics, and the like can be included in the antiseptic compositions according to the invention. Non-limiting examples of analgesics include acetylsalicylic acid, codeine, ibuprofen, acetaminophen, or tea tree oil. Non-limiting examples of anaesthetics include xylocalne, prilocaine or benzocaine.

### Preparation of the Antiseptic Compositions

The antiseptic compositions of the invention are prepared as generally outlined below. In one embodiment, the wide spectrum antiseptic component (WSAC) of the antiseptic composition is prepared, and the WSAC is then formulated if necessary depending on its intended use. Optional ingredients such as the anti-inflammatory agents when included can be added either during the preparation of the WSAC, or to the prepared WSAC.

### Preparation of the WSAC

The WSAC can be prepared as a 1× composition, or, as is known in the art, the WSAC alternatively can be prepared in more concentrated form and diluted prior to use or during formulation of the antiseptic composition for therapeutic use. Methods of preparing the WASC are known in the art, for example, as described in U.S. Pat. No. 7,338,927.

An exemplary method of preparing the WSAC includes the following steps:
dissolving one or more antimicrobial phenol compound in alcohol, with stirring, to provide an alcohol solution;
dissolving at least one or more antimicrobial agents in deionised, double-distilled water, to provide an aqueous solution;
adding one or more non-ionic detergents to the alcohol solution while continuing to stir; and
adding the aqueous solution to the alcohol solution at a sufficiently slow rate to prevent points of nucleation, to provide the WSAC.

As indicated above, where an anti-inflammatory agent or other active agent is included in the antiseptic composition, the agent can be added to the WSAC after combining the alcohol solution with the aqueous solution. Alternatively, the agent can be added to either the alcohol solution or the aqueous solution prior to combining the two solutions, depending on whether agent is soluble in aqueous solution or in alcohol.

The sequence of addition of ingredients can be varied to a certain extent from that described above, as would be known to a worker skilled in the art, depending on the solubilities of the components to be included in the WSAC. For example, if an antimicrobial agent is soluble in alcohol, and not soluble in water, it can be added to the alcohol solution instead of to the deionised, double-distilled water solution. However, the antimicrobial phenol compound should be dissolved in the alcohol prior to adding other components of the WSAC to the alcohol solution, and prior to adding the aqueous solution to the alcohol solution. The antimicrobial phenol compound should be added slowly to the alcohol to allow complete miscibility in the alcohol, and to facilitate the formation of a complex of the antimicrobial compound in free radical form.

During preparation of the WSAC, care must be taken to slowly add the aqueous solution to the alcohol solution, to avoid causing the antimicrobial phenol compound or other components to precipitate from solution.

Further, in one embodiment, double-distilled deionized water is used, providing a non-ionic environment so as to avoid points of nucleation that would cause the antimicrobial phenol to leave solution. Use of regular water may allow the antimicrobial phenol compound to leave the solution, ('points of nucleation'). As is known in the art, certain antimicrobial phenol compounds such as OPP, in free or non-salt form have a natural tendency to want to leave or form a guam thus separating from the alcohol when exposed to regular water.

The resulting WSAC can optionally be sterilized prior to use, as is known in the art. For example, the WSACs, when prepared in solution form, can be filter sterilized using, for example a 0.20 micron filter, or a 20 nm filter prior to storage. The optional step of filtration through a 0.20 micron filter ensures the WSAC is substantially free of impurities, and free of any vegetative growth which could potentially compromise its quality and efficacy.

As indicated above, the resulting antiseptic compositions according to the invention have a neutral pH. In one embodiment, the pH of the resulting antiseptic composition is within the range of 5 to 9. In one embodiment, the pH of the resulting antiseptic composition is within the range of 6.5 to 8.5. In one embodiment, the pH of the resulting antiseptic composition is within the range of 7.0 to 9.0. In one embodiment, the pH of the resulting antiseptic composition is within the range of 8 to 9. In one embodiment, the pH of the resulting antiseptic composition is within the range of 6.5 to 7.5. In one embodiment, the pH of the resulting antiseptic compositions can be adjusted to neutral pH as long as the resulting antiseptic composition still have a low ionic strength.

The antiseptic compositions according to the invention have a low ionic strength. Ionic strength of the antiseptic compositions can be calculated as is known in the art, for example by the Debye and Hückel method, in which the ionic strength, I, of a solution is a function of the concentration of all ions present in a solution.

$$I = \frac{1}{2} \sum {C}_{i} {z}_{i}^{2}$$

where Cᵢ is a molar concentration of i^{th} ion present in the solution and zᵢ is its charge. Summation is done for all charged molecules present in the solution.

Furthermore, the ionic strength of a solution can be measured as is known in the art, for example by the use of ion selective electrodes. In one embodiment, the ionic strength of the antiseptic compositions according to the invention is between about 0.0001M and 0.3M. In one embodiment, the ionic strength of the antiseptic compositions according to the invention is between about 0.0005M and 0.1M. In one embodiment, the ionic strength of the antiseptic compositions according to the invention is between about 0.001M and 0.05M. In one embodiment, the ionic strength of the antiseptic compositions according to the invention is between about 0.001M and 0.005M.

### Formulation of WSAC

The WSAC can be used as an antiseptic composition without any further formulation, or it can be formulated as a pharmaceutical composition for administration by combining it with one or more formulating agents, for example, non-toxic pharmaceutically acceptable carriers, diluents, excipients and/or adjuvants. If desired, other active agents may be included in the compositions, as noted above. Such antiseptic compositions are used in the treatment of various conditions in animals, including humans.

The pharmaceutical compositions may comprise from about 1% to about 95% of a WSAC. Compositions formulated for administration in a single dose form may comprise, for example, about 20% to about 90% of the WSAC, whereas compositions that are not in a single dose form may comprise, for example, from about 5% to about 20% of the WSAC. Non-limiting examples of unit dose forms include ampoules, vials, suppositories, pessories and capsules.

For administration to an animal, the pharmaceutical compositions can be formulated for administration by a variety of routes. For example, the compositions can be formulated for topical (including topical formulations for the oral cavity), vaginal or rectal administration or for administration by inhalation or spray.

In one embodiment, the pharmaceutical composition is formulated as a solution. In one embodiment, the pharmaceutical composition is substantially free of oil.

Pharmaceutical compositions formulated as suspensions or gels contain the WSAC in admixture with one or more suitable excipients, for example, with suspending agents, such as sodium carboxymethylcellulose, methyl cellulose, hydropropylmethylcellulose, sodium alginate, polyvinylpyrrolidone, hydroxypropyl-β-cyclodextrin, gum tragacanth and gum acacia; dispersing or wetting agents such as a naturally-occurring phosphatide, for example, lecithin, or condensation products of an alkylene oxide with fatty acids, for example, polyoxyethyene stearate, or condensation products of ethylene oxide with long chain aliphatic alcohols, for example, hepta-decaethyleneoxycetanol, or condensation products of ethylene oxide with partial esters derived from fatty acids and a hexitol for example, polyoxyethylene sorbitol monooleate, or condensation products of ethylene oxide with partial esters derived from fatty acids and hexitol anhydrides, for example, polyethylene sorbitan monooleate. The suspensions or gels may also contain one or more preservatives, for example ethyl, or n-propyl p-hydroxy-benzoate, one or more colouring agents and the like.

Pharmaceutical compositions can be formulated as oily suspensions by suspending the WSAC in a vegetable oil, for example, arachis oil, olive oil, sesame oil or coconut oil, or in a mineral oil such as liquid paraffin. The oily suspensions may contain a thickening agent, for example, beeswax, hard paraffin or cetyl alcohol. These compositions can be preserved by the addition of an anti-oxidant such as ascorbic acid.

Pharmaceutical compositions of the invention can also be formulated as oil-in-water emulsions. The oil phase can be a vegetable oil, for example, olive oil or arachis oil, or a mineral oil, for example, liquid paraffin, or it may be a mixture of these oils. Suitable emulsifying agents for inclusion in these compositions include naturally-occurring gums, for example, gum acacia or gum tragacanth; naturally-occurring phosphatides, for example, soy bean, lecithin; or esters or partial esters derived from fatty acids and hexitol, anhydrides, for example, sorbitan monoleate, and condensation products of the said partial esters with ethylene oxide, for example, polyoxyethylene sorbitan monoleate.

Various additives known in the art may be included in formulations of the composition. Examples of additives include, but are not limited to, solubilizers, skin permeation enhancers, opacifiers, preservatives (for example, anti-oxidants), gelling agents, buffering agents, emulsifiers, emollients, thickening agents, stabilizers, humectants, colorants, fragrance, and the like.

Examples of solubilizers include, but are not limited to, the following: hydrophilic ethers such as diethylene glycol monoethyl ether (ethoxydiglycol, available commercially as Transcutol™) and diethylene glycol monoethyl ether oleate (available commercially as Softcutol™); polyethylene castor oil derivatives such as polyoxy 35 castor oil, polyoxy 40 hydrogenated castor oil, etc.; polyethylene glycol, particularly lower molecular weight polyethylene glycols such as PEG 300 and PEG 400, and polyethylene glycol derivatives such as PEG-8 caprylic/capric glycerides (available commercially as Labrasol™); alkyl methyl sulfoxides such as DMSO; pyrrolidones such as 2-pyrrolidone and N-methyl-2-pyrrolidone; and DMA. Many solubilizers can also act as absorption enhancers. A single solubilizer may be incorporated into the formulation, or a mixture of solubilizers may be incorporated therein.

Suitable emulsifiers and co-emulsifiers are known in the art. Emollients include, for example, propylene glycol, glycerol, isopropyl myristate, polypropylene glycol-2 (PPG-2) myristyl ether propionate, and the like.

Other pharmaceutical compositions and methods of preparing pharmaceutical compositions are known in the art and are described, for example, in “Remington: The Science and Practice of Pharmacy” (formerly “Remington Pharmaceutical Sciences”); Gennaro, A., Lippincott, Williams & Wilkins, Philadelphia, Pa. (2000).

### Testing of the Antiseptic Compositions

The antiseptic compositions according to the invention are capable of killing microbial cells. In one embodiment, the antiseptic compositions are capable of killing bacteria, fungi, protozoans and viruses rapidly. Antiseptic compositions comprising an anti-inflammatory agent are not only capable of killing these microbial cells, but are also capable of reducing inflammation. The antiseptic compositions exhibit minimal toxicity and irritation, and thus are suitable for topical administration to a subject for the treatment and/or prevention of dermal, genital, cuticle or mucosal infections. The ability of the antiseptic compositions according to the present invention to kill, or inhibit the growth of bacteria, fungi, protozoans and/or viruses, as well as the toxicity and irritation to a subject can be tested using standard techniques known in the art. Although the methods described herein refer to testing of candidate antiseptic compositions, the same methods can be used to test the candidate WSACs or candidate combinations of WSAC and other active agents (such as anti-inflammatory agents). The candidate antiseptic compositions can be tested in vitro and/or in vivo to determine their efficacy, toxicity, and irritant properties. Exemplary methods of testing these candidate antiseptic compositions are provided below and in the Examples included herein. One skilled in the art will understand that other methods of testing the antiseptic compositions are known in the art and are also suitable for testing candidate antiseptic compositions.

### A. In Vitro Testing

In vitro methods of determining the ability of candidate antiseptic compositions to kill or inhibit the growth of microbial cells are well-known in the art. In general, these methods involve contacting a culture of the cells of interest with various concentrations of the candidate antiseptic compositions and monitoring the growth of the cell culture relative to an untreated control culture. A second control culture comprising cells contacted with a known anti-microbial agent may also be included in such tests, if desired.

For example, the ability of a candidate antiseptic composition to inhibit the growth of microbial cells can readily be determined by measurement of the minimum inhibitory concentration (MIC) for the antiseptic composition. The MIC is defined as the lowest concentration that inhibits growth of the organism to a pre-determined extent. For example, a MIC₁₀₀ value is defined as the lowest concentration that completely inhibits growth of the organism, whereas a MIC₉₀ value is defined as the lowest concentration that inhibits growth by 90% and a MIC₅₀ value is defined as the lowest concentration that inhibits growth by 50%. MIC values are sometimes expressed as ranges, for example, the MIC₁₀₀ for an antiseptic composition may be expressed as the concentration at which no growth is observed or as a range between the concentration at which no growth is observed and the concentration of the dilution which immediately follows.

Anti-bacterial MICs for candidate antiseptic compositions can be measured using a broth macro- or microdilution assay (see Amsterdam, D. (1996) “Susceptibility testing of antimicrobials in liquid media,” pp. 52-111. In Loman, V., ed. Antibiotics in Laboratory Medicine, 4th ed. Williams and Wilkins, Baltimore, Md.). A standardised anti-bacterial susceptibility test is provided by the National Committee for Clinical Laboratory Standards (NCCLS) as NCCLS, 2000; document M7-A58.

In the classical broth microdilution method, the candidate antiseptic composition is diluted in culture medium in a sterile, covered 96-well microtiter plate. An overnight culture of a single bacterial colony is diluted in sterile medium such that, after inoculation, each well in the microtiter plate contains an appropriate number of colony forming units (CFU)/ml (typically, approximately 5×10⁵ CFU/ml). Culture medium only (containing no bacteria) is also included as a negative control for each plate and known antibiotics are often included as positive controls. The inoculated microtiter plate is subsequently incubated at an appropriate temperature (for example, 35° C.-37° C. for 16-48 hours). The turbidity of each well is then determined by visual inspection and/or by measuring the absorbance, or optical density (OD), at 595 nm or 600 nm using a microplate reader and is used as an indication of the extent of bacterial growth.

Techniques for determining anti-fungal MIC values for candidate antiseptic compositions are similar to those outlined above for anti-bacterial MICs and include both macrodilution and microdilution methods (see, for example, Pfaller, M. A., Rex, J. H., Rinaldi, M. G., Clin. Infect, Dis., (1997) 24:776-84). A standardised anti-fungal susceptibility test method, NCCLS M27-T, has been proposed by the NCCLS (see, Ghannoum, M. A., Rex, J. H. and Galgiani J. N., J. Clin. Microbiol., (1996) 34:489-495; Pfaller, M. A. and Yu, W. L., Infect. Dis. Clin. North Amer., (2001) 15:1227-1261).

In accordance with one embodiment of the invention, an antiseptic composition is considered to have an anti-microbial effect against a given micro-organism when used alone when the MIC of the antiseptic composition for complete inhibition of growth of the organism is less than about 75 μg/ml. In one embodiment, the antiseptic composition has a MIC less than about 50 μ/ml for the relevant micro-organism. In another embodiment, the antiseptic composition has a MIC of less than about 35 μg/ml. In other embodiments, the antiseptic composition has a MIC of less than about 25 μg/ml, less than about 16 μg/ml and less than about 12.5 μg/ml for the relevant micro-organism.

Anti-microbial effects may also be expressed as the percentage (%) inhibition of growth of a given micro-organism over a pre-determined period of time by treatment with a single concentration of a candidate antiseptic composition. This method provides a rapid method of assessing the ability of an antiseptic composition to inhibit microbial growth, for example, prior to conducting more in-depth tests, such as MIC determinations or in vivo testing.

### Antibacterial Activity

The ability of the antiseptic compositions to kill or inhibit the growth of bacteria can be tested using methods well-known in the art, including the broth dilution method described above. Methods and protocols for testing compositions against specific bacteria can be found, for example, in Official Methods of Analysis of the AOAC, 15^{th} Ed., Arlington Va. 22201, USA (Association of Official Analytical Chemists (AOAC), Inc. 1990), Designation: E 1054-91 “Practices for Evaluation Inactivators of Antimicrobial Agents Used in Disinfectant, Sanitizer, Antiseptic, or Preserved Products” (American Society for Testing and Materials (ASTM), 1991). As is also known in the art, in vitro Time-Kill evaluations can be performed using a modification of the methods described in the Draft European Standard, prEN 12054, “Chemical Disinfectants and Antiseptics—Products for Hygienic and Surgical Handrub and Handwash—Bactericidal Activity—Test Method and Requirements (1995).” The ability of candidate antiseptic compositions to kill mycobacteria can be assessed using methods based on those described in the European Standard, EN 14348 “Chemical Disinfectants and Antiseptics—Quantitative Suspension Test for the Evaluation of Mycobatericidal Activity of Chemical Disinfectants in the Medical Area Including Instrument Disinfectants—Test Method and Requirements (2005)”, Test methods by the American Standard Test Methods (ASTM) Designation: E 1054-91, and AOAC methods (1990) which are modified and used for evaluation of antimicrobial effectiveness of a disinfectant.

An exemplary method of testing antibacterial activity of candidate antiseptic compositions is described as follows. An inoculum of test bacteria (about 10⁵ cfu/mL, for example, as is known in the art, the number of cfu in the inoculum may vary depending on the species of bacteria being tested) is coated onto sterile glass coverslips, and air dried. Coverslips containing the inoculum are contacted with the candidate antiseptic compositions for varying periods of time, and the number of surviving bacteria is determined, by transferring each coverslip to an aliquot of appropriate broth, incubating the broth and coverslips, and determining the number of surviving bacteria in the broth. The number of surviving bacteria on coverslips that have not been exposed to candidate antiseptic compositions (positive control) is also determined. For comparison, the candidate antiseptic compositions can be tested against known reagents with anti-bacterial activity, such as Septol (at 1%, for example), ethanol (at 70%, for example), and/or phenol (at 1/60, for example).

Additional methods that may be used include the log reduction test, proliferation testing, the AOAC use dilution test, or the zone of inhibition test.

### Anti-Viral Activity

Candidate antiseptic compositions can be tested to determine their anti-viral activity as is known in the art. For example, a Virucidal Suspension Test (In-Vitro Time-Kill) can be performed using a modification of the methods described in the European Standard, EN 14476 “Chemical Disinfectants and Antiseptics—Virucidal Quantitative Suspension Test for Chemical Disinfectants and Antiseptics Used in Human Medicine (2005)”

The anti-viral activity of candidate antiseptic compositions can determined using a procedure similar to that described above for testing anti-bacterial activity, but using an inoculum of virus on the coverslips, with other minor modifications to the procedure in order to quantitate the number of surviving viruses.

### Anti Fungal Activity

The ability of candidate antiseptic compositions to kill or inhibit the growth of fungi can also be determined as is known in the art, including the broth dilution method described above. For example, an In-vitro Time-Kill evaluation can be performed using a modification of the methods described in the Draft European Standard, prEN 13624, “Chemical Disinfectants and Antiseptics—Quantitative Suspension Test for the Evaluation of Fungicidal Activity of Chemical Disinfectants for Instruments Used in the Medical Area—Test Method and Requirements (2003) and “CAN/CGSB-2.161-97, Assessment of Efficacy of Antimicrobial Agents for Use on Environmental Surfaces and Medical Devices.”

In general, these methods involve contacting a culture of the fimgal cells of interest with various concentrations of the candidate antiseptic compositions and monitoring the growth of the cell culture relative to an untreated control culture. A second control culture comprising cells contacted with a known anti-fungal agent may also be included in such tests, if desired.

As an example, the anti-fungal activity of candidate antiseptic compositions can determined using a procedure similar to that described above for testing anti-bacterial activity, but using an inoculum of fungi on the coverslips, with other minor modifications to the procedure in order to quantitate the number of surviving fungal cells.

### Anti-Protozoan Activity

Wide spectrum antimicrobial agents such as chlorhexidine gluconate, chlorhexidine diacetate, polyhexamethylene biguanide, benzalkonium chloride are known to be effective against protozoans in addition to other microorganisms (see for example: McDonnell G. & Russell A. D. 1999 Antiseptics and Disinfectants: Activity, Action, and Resistance. Clin. Microbiol. Rev. 12(1): 147-179; Khunkitti W, Hann A C, Lloyd D, Furr J R, Russell A D. 1998. Biguanide-induced changes in Acanthamoeba castellanii: an electron microscopic study. J Appl Microbiol. 84(1):53-62; Lloyd D, Turner N A, Khunkitti W, Hann A C, Furr J R, Russell A D. 2001. Encystation in Acanthamoeba castellanii: development of biocide resistance. J Eukaryot Microbiol. 48(1):11-6; Turner N A, Russell A D, Furr J R, Lloyd D. 2000. Emergence of resistance to biocides during differentiation of Acanthamoeba castellanii. J Antimicrob Chemother. 46(1):27-34). Thus it can be predicted that antiseptic compositions according to the invention would be useful in the treatment of protozoan-related infections.

The protozoicidal effect of the compositions can be determined using standard techniques known in the art. For example, the ability of the compositions to kill representative protozoa such as Paramecium caudatum, Euglena spp., Tetrahymena pyriformis, Polytomella papillata, Amoeba proteus and Chaos spp. (all commercially available, for example, from Carolina Biological Supply Company, Burlington, N.C.) and Trichimoas vaginalis (available from the American Type Culture Collection (ATCC)) can be tested as follows. First different concentrations of the composition are prepared in an appropriate volume of medium. Appropriate media for the maintenance of protozoa are known in the art and can be obtained commercially, for example from Carolina Biological Supply Company. The dilutions are poured into multiwell dishes and an appropriate number of protozoa, for example, 10 microorganisms, are added to each well and incubated at an appropriate temperature (generally between about 22° C. and about 28° C.). The protozoa in the plates are observed periodically under a microscope over a period of about 24 hours to determine when all the protozoa have been killed. The protozoa are presumed to be killed when all of the protozoa in the well have stopped moving. Results are generally expressed as the time in minutes for a 100% kill. In order to determine whether the protozoa have been killed rather than only temporarily inactivated, the organisms are centrifuged, the supernatant removed, and the protozoa placed into fresh medium. When there is no revival of the organisms, it can be concluded that they are dead rather than only temporarily inactivated.

Other methods are known in the art, such as determination of minimum lethal concentrations (MLCs) and kill rates (see, for example, Nix D. E., Tyrell R. and Müller R M.; Antimicrob. Agents Chemother. 1995, 39(8):1848-1852).

### Kill Rates

In one embodiment, the antiseptic compositions according to the present invention are able to rapidly kill or inhibit the growth of microorganisms such as bacteria, fungi, viruses, and/or protozoans. The speed at which the antiseptic compositions are able to kill can be measured in vitro by the tests indicated above and described as a kill rate, i.e. the kill rate is defined as the length of time it takes for the antiseptic composition to kill all the microorganisms present in a test sample. As is known in the art, the kill rate can vary depending on the assay conditions used. The following kill rates are measured according to the assays described above and in accordance with the assays described in the Example section of the application. Thus, in one embodiment, the antiseptic compositions have a kill rate of less than 15 minutes. In one embodiment, the antiseptic compositions have a kill rate of less than 10 minutes. In one embodiment, the antiseptic compositions have a kill rate of less than 5 minutes. In one embodiment, the antiseptic compositions have a kill rate of less than 3 minutes. In another embodiment, the antiseptic compositions have a kill rate of between about 1 to 5 minutes.

It should be understood that although the antiseptic compositions of the invention have the kill rates described above when tested in vitro, the efficacy of the antiseptic compositions when used in a therapeutic context may only be evident over longer periods of time, such as for example in terms of hours, days, or weeks, depending on the severity of the infection, and the treatment regime used.

### B. In Vivo Testing

The antimicrobial activity of the antiseptic compositions according to the invention can also be tested in vivo using standard techniques. A number of animal models are known in the art that are suitable for testing the antimicrobial activity of the antiseptic compositions and are readily available, such as “EN 1499, Chemical Disinfectants and Antiseptics-Hygienic Handwash-Test Method and Requirements”, “prEN 1500, Chemical Disinfectants and Antiseptics-Hygienic Handrub- Test Method and Requirements”, and “EN 12791, Chemical Disinfectants and Antiseptics-Surgical Hand Disinfection-Test Method and Requirements”.

Examples of mouse models for topical treatment of skin infections are the burnt skin model (Akiyama, et al. 1994. Staphylococcus aureus infection on experimental croton oil-inflamed skin in mice. J. Dermatol. Sci. 8:1-10; Stieritz, et al., 1982. A burned mouse model to evaluate anti-Pseudomonas activity of topical agents. J. Antimicrob. Chemother. 9:133-140; and Yarnakawa, et al., 2002. In vitro and in vivo antibacterial activity of T-3912, a novel non-fluorinated topical quinolone. J. Antimicrob. Chemother. 49:455-465) and the skin suture-wound model (Boon, et al., 1987. Response of Streptococcus pyogenes to therapy with amoxicillin or amoxicillin-clavulanic acid in a mouse model of mixed infection caused by Staphylococcus aureus and Streptococcus pyogenes. Antimicrob. Agents Chemother. 31:1204-1209. and Gisby, J., and J. Bryant. 2000. Efficacy of a new cream formulation of mupirocin: comparison with oral and topical agents in experimental skin infections. Antimicrob. Agents Chemother. 44:255-260). In the burnt skin model, the skin of the mouse is traumatized by burning and bacteria are introduced into the skin by injection in the traumatized skin area. Candidate antiseptic compositions can be applied to the traumatized area in order to determine their efficacy. In the skin suture-wound model a bacterium-impregnated nylon suture is implanted into an artificial wound (such as a scalpel incision through all skin layers) which is then sewn or stapled shut. Candidate antiseptic compositions are applied to the wound to test their efficacy. For both models, after a suitable period of time, the animal is killed, and the infected area of the skin is cut out, and the number of bacteria in the sample is assayed by standard methods.

An additional skin infection model has been described by Kugelberg et al., in Establishment of a Superficial Skin Infection Model in Mice by Using Staphylococcus aureus and Streptococcus pyogenes. Antimicrobial Agents and Chemotherapy, August 2005, p. 3435-3441, Vol. 49. In this model, the skin barrier is disrupted by partial removal of the epidermal layer. Infection is established by application of bacteria to this site. Candidate antiseptic compositions can be administered topically to the site to determine their efficacy. Efficacy is determined by excising and homogenizing the test area after a suitable period of time, for example 3 to 5 days, and then plating the homogenate to determine the number of bacteria present.

### Toxicity

The candidate antiseptic compositions can be tested to determine their toxicity according to methods known in the art, and guidelines established by various regulatory bodies such as for example, the United States Environmental Protection Agency's Office of Prevention, Pesticides, and Toxic Substances (OPPTS), such as “Acute Dermal Toxicity Study in Rats—Limit Test, Health Effects Test Guidelines, OPPTS 870.1200 (1998)”, “Primary Eye Irritation Study in Rabbits, Health Effects Test Guidelines, OPPTS 870.2400 (1998)”, “Primary Skin Irritation Study in Rabbits, Health Effects Test Guidelines, OPPTS 870.2500 (1998)” and “Dermal Sensitization Study in Guinea Pigs (Buehler Method) Health Effects Test Guidelines, OPPTS 870.2600 (1998)”.

It is important that the antiseptic compositions of the invention exhibit low toxicity in vivo. Toxicity tests for potential drugs are well-known in the art (see, for example, Hayes, A. W., ed., (1994), Principles and Methods of Toxicology, 3^{rd} ed., Raven Press, NY; Maines, M., ed., Current Protocols in Toxicology, John Wiley & Sons, Inc., NY).

In accordance with one embodiment of the present invention, an antiseptic composition according to the invention for use in vivo shows both good anti-microbial activity and low or no toxicity at the concentration at which it would be administered as an anti-microbial treatment.

In vitro acute toxicity testing of an antiseptic composition can be performed using mammalian cell lines (see, for example, Ekwall, B., Ann. N.Y. Acad. Sci., (1983) 407:64-77). Selection of an appropriate cell line is dependent on the potential application of the candidate antiseptic composition and can be readily determined by one skilled in the art.

In vivo toxicity testing can be performed by standard methodology, for example, by injecting varying concentrations of the candidate antiseptic composition into an appropriate animal model. The antiseptic composition can be injected once, or administration can be repeated over several days. The toxic effects of the antiseptic composition can be evaluated over an appropriate time period by monitoring the general health and body weight of the animals. After the completion of the period of assessment, the animals can be sacrificed and the appearance and weight of the relevant organs determined.

For example, the candidate antiseptic compositions can be tested in an acute inhalation toxicity model in rats to determine the potential for the candidate antiseptic composition to produce toxicity from single or multiple exposures via the inhalation route. Briefly, in this model healthy rats are exposed to the candidate antiseptic composition in an inhalation chamber for a suitable period of time, such as for example 4 hours. The rats are observed for mortality, signs of gross toxicity, and behavioral changes over an observation period after exposure. A suitable observation period is 14 days, for example. Body weights of the rats can be measured at intervals, and necropsies can be performed at the end of the observation period in order to determine whether the antiseptic composition is toxic. In one embodiment, the LC₅₀ of the antiseptic composition is greater than 2 mg/L when measured in an acute inhalation toxicity model.

As another example, the oral toxicity of candidate antiseptic compositions can also be determined in a rat acute toxicity model. In this model, the toxicity of single or multiple oral doses of a candidate antiseptic composition can be determined over an observation period after administration to a healthy rat. The antiseptic composition can be administered, for example, by oral gavage. As indicated above, mortality, behavioral changes and signs of gross mortality in the rats are monitored over the observation period to determine the toxicity of the candidate antiseptic composition. In one embodiment, the LD₅₀ of the antiseptic composition is greater than 1000 mg/kg body weight when measured in this type of model.

### Irritation

Candidate antiseptic compositions can be tested to determine whether they are irritants. Methods of testing candidate compositions to determine their potential to be irritants are known in the art, and include use of models such as for example, primary skin irritation in rabbits, primary eye irritation in rabbits, dermal sensitization in guinea pigs, and a pig skin irritation study. Some of these models are described briefly below.

Testing of candidate antiseptic compositions in the primary skin irritation model in rabbits can be carried out as follows. The candidate antiseptic composition is applied to an area of exposed skin (test site) on the rabbit for a period of time (for example 4 hours), and then washed away. The test site is then monitored at intervals over a period of time such as, for example 72 hours, according to the Draize scoring system (Draize, et al., J. Pharmacol. Exp. Ther. (1944) 82:377-390) to identify signs of irritation, such as erythema and edema. The rats can also be observed further to identify additional signs of irritation and toxicity including, gross evaluation of skin and fur, eyes and mucous membranes, tremors, convulsions, salivation, diarrhea and coma, for example.

In the primary eye irritation model, the candidate antiseptic composition is instilled into one eye of a rabbit (the other eye remains untreated and serves as a control). In one group of rabbits, the candidate antiseptic composition is then rinsed out of the eye, and in another group, the candidate antiseptic composition remains in the eye. Ocular irritation is then evaluated at intervals of hours or days over a suitable period of time (for example, 21 days). The symptoms of irritation such as corneal opacity, iritis, and conjunctivitis are measured and scored using the Draize scoring system (Draize, J. H. et al., supra).

In the guinea pig dermal sensitization model, the antiseptic compositions can be tested in the following way. The fur of the animals is removed from the test area and the test substance is applied to one side of the test area at a suitable frequency (for example, once a week), for a suitable period of time (for example 3 weeks). Local reactions or erythema are determined using a standard scoring system.

### Administration and Delivery

The antiseptic compositions according to the invention are administered in an amount effective to achieve the intended purpose. Thus the term “therapeutically effective dose” refers to the amount of the antiseptic composition that improves the status of the subject to be treated, for example, by ameliorating the symptoms of the disease or disorder to be treated, preventing the disease or disorder, or altering the pathology of the disease. Determination of a therapeutically effective dose of an antiseptic composition is well within the capability of those skilled in the art. For example, the therapeutically effective dose can be estimated initially either in cell culture assays, or in animal models, such as those described herein. Animal models can also be used to determine the appropriate concentration range and route of administration. Such information can then be used to determine useful doses and routes for administration in other animals, including humans using standard methods known in those of ordinary skill in the art.

Therapeutic efficacy and toxicity can also be determined by standard pharmaceutical procedures such as, for example, by determination of the median effective dose, or ED₅₀ (i.e. the dose therapeutically effective in 50% of the population) and the median lethal dose, or LD₅₀ (i.e. the dose lethal to 50% of the population). The dose ratio between therapeutic and toxic effects is known as the “therapeutic index,” which can be expressed as the ratio, LD₅₀/ED₅₀. The data obtained from cell culture assays and animal studies can be used to formulate a range of dosage for human or animal use. The dosage contained in such compositions is usually within a range of concentrations that include the ED₅₀ and demonstrate little or no toxicity. The dosage varies within this range depending upon the dosage form employed, sensitivity of the subject, and the route of administration and the like.

The exact dosage to be administered to a subject can be determined by the practitioner, in light of factors related to the subject requiring treatment. Dosage and administration are adjusted to provide sufficient levels of the antiseptic composition and/or to maintain the desired effect. Factors which may be taken into account when determining an appropriate dosage include the severity of the disease state, general health of the subject, age, weight, and gender of the subject, diet, time and frequency of administration, drug combination(s), reaction sensitivities, and tolerance/response to therapy. Dosing regimens can be designed by the practitioner depending on the above factors.

The antiseptic compositions according to the invention can be delivered using a variety of techniques. These techniques may be varied depending on the subject to be treated. Typically, the compositions are delivered to the skin and/or mucosal tissue in a manner that allows them to penetrate into the skin and/or mucosal tissue, as opposed to through the tissue into the blood stream. This concentrates the compositions locally at the site in need of treatment. This delivery can be accomplished by spraying, dipping, wiping, dropping, pouring, toweling, inhaling, or the like, onto the area to be treated.

The antiseptic compositions according to the invention may be provided as a formulation suitable for delivery depending on the subject to be treated. For example, the antiseptic compositions according to the invention may be provided as a formulation suitable for delivery to mammalian tissue such as, for example, skin and/or mucosal surfaces. Suitable formulations can include, but are not limited to, creams, liquid creams, gels, foams, ointments, lotions, balms, waxes, sticks, granules, salves, solutions, suspensions, dispersions, water in oil or oil in water emulsions, microemulsions, pastes, powders, oils, lozenges, boluses, aerosol sprays, on sponges or cotton applicators, or as a solution or a suspension in an aqueous liquid, a non-aqueous liquid, an oil-in-water emulsion, or a water-in-oil liquid emulsion and the like. In one embodiment of the invention, the antiseptic compositions are formulated for topical administration.

In one embodiment, the antiseptic compositions may be sprayed from a pressurized container. The pressure may be supplied by an external means such as squeezing the container, through the use of a mechanical pump, or with the use of a propellant. Suitable propellants include chlorofluorocarbons (CFCs), hydrochlorofluorocarbons (HCFCs), hydrofluorocarbons (HFCs), hydrofluoroethers (HFEs), perfluorinated alkanes, and (C1-C5) alkanes as well as nitrous oxide and dimethyl ether.

If delivered as a foam, the composition may be dispensed from an aerating dispenser such as the F2 Finger Pump Foamer available from Air Spray International Pompano Beach, Fla. Alternatively, the foam may be generated using a suitable propellant such as those described above.

For very high viscosity formulations the composition may be delivered in essentially a solid dosage form by placing the composition in or on the tissue to be treated. For example, a small suppository type delivery could be placed into the anterior nares for eradication of Staphylococcus sp.

Various other modes of administration can be used as well known to one of skill in the art depending on the desired location for contact of the antimicrobial compositions of the present invention. For example, afflictions of the middle ear (e.g., otitis media or infection of the middle ear) may be treated with the antiseptic compositions according to the invention by administration through the nose and into the Eustachian tubes or they can be instilled directly into the middle ear through the tympanic membrane. The formulations may traverse the tympanic membrane with the aid of a syringe or do so by diffusion. Penetration enhancers may be used to enhance diffusion across the tympanic membrane.

For application to skin or mucosal tissue, for example, the antiseptic compositions may be applied directly to the tissue from a collapsible container such as a flexible tube, blow/fill/seal container, pouch, capsule, etc. In this embodiment, the primary container itself is used to dispense the antiseptic composition directly onto the tissue or it can be used to dispense the composition onto a separate applicator. For example, for delivery to the nose or other topical tissue, the antiseptic composition could be dispensed directly from a tube and spread by a number of means including squeezing the outside of the nose together repeatedly, wiping with the tip of the tube or with a separate device such as a spatula, cotton, rayon, or other natural or synthetic based fiber swab.

Other application devices may also be suitable including applicators with foam tips, brushes, and the like. Importantly, the applicator must be able to deliver the requisite amount of the antiseptic composition to the tissue. Therefore, in most instances applicator devices such as webs and swabs are coated on the applicator web at greater than 50% by weight of the dry web and preferably in excess of 100% by weight of the dry web (on a swab, this would include the weight only of the web).

An antimicrobial composition may be applied to a mucosal surface with the use of a delivery device such as cervical caps, diaphragms and solid matrices such as tampons, cotton sponges, cotton swabs, foam sponges, and suppositories. Accordingly, compositions of the present invention can also be incorporated in or delivered from cloth, sponges, paper products (such as, for example, paper towels, towelettes, and wipes), tampons, undercast padding, and dental floss, for example.

In some embodiments, an applicator may be used to place the device and/or antiseptic composition in the proper location, for example, on the mucosal surface of a vagina, nasal cavity, rectum, or the like. Examples of such applicators include, for example, cardboard or plastic tube applicators commonly used for inserting tampons or suppositories.

The compositions of the present invention can be delivered from various substrates for delivery to the tissue. For example, the compositions can be delivered from a wipe or pad which when contacted to tissue will deliver at least a portion of the composition to the tissue. For application to nasal cavities the compositions may be provided by a non-woven swab such as a “Q-tip” brand cotton swab, into a foam tip applicator, and the like. The substrate may be used to deliver the composition essentially instantaneously or may be left in contact with the tissue. For example, a substrate in a tubular form could be delivered to the anterior nares using a suitable applicator and left in the anterior nares. The annular nature of the device is designed to allow delivery of the active while allowing the patient to freely breathe through the nose.

In one embodiment, antiseptic compositions of the invention can be coated onto medical devices that contact skin, mucous membranes, wounds, for example. Examples of such devices include catheters such as urinary tract catheters and vascular access catheters.

It is contemplated that the antiseptic compositions of the invention can be used with delivery systems designed to deliver the composition through the skin or mucosa to treat dermal, mucosal, cuticle or genital conditions that are below the surface of the skin or mucosa. Examples of such delivery systems include liposomes, for example. Other examples of such delivery systems include the use of absorption enhancers, such as dimethylsulphoxide (DMSO) the terpene derivatives linalool, alpha terpineol, carvacrol, limonene, menthone, and eugenol or the polyphenol, Phloretin, active transfer methods such as iontophoresis; sonophoresis and electroporation and use of vasodilators, such as nitroglycerin, nicotine, and caffeine.

### Uses

The antiseptic compositions of the invention are used in the treatment of dermal, mucosal, cuticle or genital infections that result from microbial infections (caused by bacteria, viruses, protozoans or fungi) and that may or may not be accompanied by inflammation. In one embodiment, the antiseptic compositions can be used to prevent secondary infections in a subject suffering from a non-infectious condition. For example, the antiseptic compositions can be used to treat eczema with and without secondary bacterial, viral or fungal infections, dermatitis with and without secondary bacterial, viral or fungal infections and inflamed insect bites with or without secondary bacterial, viral or fungal infections.

In one embodiment, the antiseptic compositions can be used to treat dermal, mucosal, cuticle or genital infections such as, but not limited to, Tinea pedis (athlete's foot), Tinea corporis (ring worm), Tinea versicolar, Tinea cruris (jock itch), Tinea manuum (hand and palm infections), and impetigo.

In one embodiment, the antiseptic compositions can be used to treat dermal, mucosal, cuticle or genital conditions such as acne (Propionibacterium acnes), cold sores (Herpes simplex I), Herpes zoster (shingles), seborrheic dermatitis, genital warts, or plantar warts, for example.

In one embodiment the antiseptic composition is used to treat dermal, mucosal, cuticle or genital conditions that are known in the art as weeping infections, such as, for example, Staphylococcus infections.

In one embodiment, the antiseptic composition is used to treat dermal, mucosal, cuticle or genital conditions caused by bacteria of the genera Staphylococcus, Salmonella, Pseudomonas, Enterococcus, Escherichia, and Proteus. Such conditions include post-surgical wound infections and vulvo-vaginal infections.

In one embodiment, the antiseptic composition is used to treat dermal, mucosal, cuticle or genital conditions caused by fungi of the genera Trichophyton, Candida, and Aspergillus. Such conditions include Tinea pedis, vulvo-vaginal infections and paronychia.

In one embodiment, the antiseptic composition is used to treat dermal, mucosal, cuticle or genital conditions caused by viruses of the genera Herpes. Such conditions include Herpes zoster, herpes genitalis, oral herpetic infections, and aphthous ulcers.

In one embodiment, the antiseptic composition is used to treat cuticle infections. Such cuticle infections include onchomycosis.

In one embodiment, the antiseptic composition is used to treat dermal infections. Such dermal infections include cellulitis, furuncles, carbuncles, ulcerations, planters warts and infected abrasions.

In one embodiment, the antiseptic compositions according to the invention can be used in veterinary applications, including the treatment of dermal infections such as ringworm, infections of the paws and hooves and ear infections.

The efficacy of treatment of the above-noted dermal, mucosal, cuticle or genital conditions is determined by monitoring the symptoms that accompany the condition. For example, successful treatment of the condition could be indicated by an improvement in the condition of the tissue at the site of the affliction, for example, by either by softening or thinning of the skin, or decreasing the fluid or exudate at the site of the affliction, disappearance of a rash, reduction in pain, fever (if present) and fatigue, improved libido, improved appetite, improved function of the affected area and the like.

As is known in the art, the appropriate treatment regime for dermal, mucosal, cuticle and genital conditions will vary from patient to patient, depending on the patient's responsiveness to the treatment and the severity of the condition. For example, the severity of the condition may dictate the amount of the antiseptic composition to be used, the frequency of application of the antiseptic composition, the components to be included in the antiseptic composition, and the overall length of treatment with the antiseptic composition. For example, an antiseptic composition comprising a particular anti-inflammatory agent may not be as effective a treatment as an antiseptic composition comprising a different anti-inflammatory agent, depending on the condition to be treated, or depending on the patient to be treated. The determination of an appropriate treatment regime is well within the knowledge of one skilled in the art.

### Pharmaceutical Kits

The invention additionally provides for therapeutic kits or packs containing the antiseptic composition or a pharmaceutical composition comprising the antiseptic composition for use in the treatment of dermal, genital, cuticle or mucosal infections. It is further contemplated that the WSAC and other active agents, such as anti-inflammatory agents of the antiseptic composition may be provided in the kit as separate components and mixed prior to use, or may be provided premixed. Individual components of the kit can be packaged in separate containers, associated with which, when applicable, can be a notice in the form prescribed by a governmental agency regulating the manufacture, use or sale of pharmaceuticals or biological products, which notice reflects approval by the agency of manufacture, use or sale for human or animal administration. The kit can optionally further contain one or more other therapeutic agents for use in combination with the antiseptic compositions of the invention. The kit may optionally contain instructions or directions outlining the method of use or dosing regimen for the antiseptic compositions and/or additional therapeutic agents.

When one or more components of the kit are provided as solutions, for example an aqueous solution, or a sterile aqueous solution, the container means may itself be an inhalant, syringe, pipette, eye dropper, or other such like apparatus, from which the solution may be administered to a subject or applied to and mixed with the other components of the kit.

The components of the kit may also be provided in dried or lyophilised form and the kit can additionally contain a suitable solvent for reconstitution of the lyophilised components. Irrespective of the number or type of containers, the kits of the invention also may comprise an instrument for assisting with the administration of the composition to a patient. Such an instrument may be an inhalant, syringe, pipette, forceps, measured spoon, eye dropper or similar medically approved delivery vehicle.

To gain a better understanding of the invention described herein, the following examples are set forth. It will be understood that these examples are intended to describe illustrative embodiments of the invention and are not intended to limit the scope of the invention in any way.

### EXAMPLES

### Example 1

### Preparation of Antiseptic Composition 1 (ASC-1)

An antiseptic composition according to the present invention (ASC-1) was prepared containing constituent components in the concentrations listed in the following table:

TABLE 1
Concentration of components in ASC-1

| | | Final concentration |
|---|---|---|
| | | of each ingredient in |
| Component | Amount | ASC-1 (w/w) |
| SDAG-3 ethanol, | 73.68 grams | 70% |
| (95%, containing 5% | | |
| water and 700 mg/100 | | |
| L Bitrex ™) | | |
| o-phenylphenol (OPP) | 0.28 grams | 0.28% |
| Benzalkonium chloride | 0.40 grams | 0.20% |
| (BZK) (50% solution | | |
| in water) | | |
| Chlorhexidine | 0.01 grams | 0.01% |
| gluconate (CHG) | | |
| Nonoxynol-9 | 0.05 grams | 0.05% |
| Deionised, double- | 29.36 grams | 29.36% |
| distilled water | | |
| (including water | | |
| contained in 95% | | |
| Ethanol and 50% BZK | | |
| solution) | | |
| Lemon fragrance No. 431 | 0.1 grams | 0.10% |

ASC-1 was prepared by:
adding the SDAG-3 ethanol to a grounded mixing tank;
adding OPP to the mixing tank containing ethanol slowly until the OPP was fully dissolved. Once fully dissolved, continued mixing was carried out for an additional 15 minutes;
adding and continually mixing the fragrance, chlorhexidine gluconate and nonoxynol-9 to the grounded mixing tank;
bleeding double distilled deionized water into the mixing tank at a sufficiently slow rate to prevent shock to the solution in the tank and avoid points of nucleation,
adding and mixing benzalkonium chloride to the mixing tank, with continued mixing for an additional 30 minutes.

The above steps were carried out in the order listed above, and the mixture was then filtered using a 0.20 micron filter and stored in sterilized bottles.

### Example 2

### Preparation of Antiseptic Composition 2 (ASC-2)

An antiseptic composition including hydrocortisone as an anti-inflammatory agent (ASC-2) and formulated for topical use was prepared containing the components as listed in the following table:

TABLE 2
Components in ASC-2
Component

SDAG-3 ethanol, 95%, containing 700
mg/100 L Bitrex ™)
o-phenylphenol (OPP)
Benzalkonium chloride (BZK)
Chlorhexidine gluconate (CHG)
Nonoxynol-9
Deionised, double-distilled water
Lemon fragrance No. 431
Hydrocortisone
Hydroxypropylcellulose

As indicated in Table 2, ASC-2 contained 2.00% hydrocortisone and was prepared as follows. ASC-1 was prepared as described in Example 1. Approximately 900 ml of ASC-1 was poured into a 1 L glass beaker. Hydrocortisone USP powder (20 g) was added to the solution and allowed to dissolve with gentle stirring.

In order to formulate the resulting solution as a geI for topical use, hydroxypropylcellulose USP (20 g) was then added slowly to the solution with constant and vigorous stirring. The final concentration of hydroxypropylcellulose USP in the formulation was 2.00%. Once the mixture was gelled the volume of the formulated composition was adjusted to 1000 ml with an additional volume of ASC-1 and mixed for another 1 minute until the whole mixture was homogenous, to obtain ASC-2. Individual plastic tubes were each filled with 15 g of ASC-2 using a 30 ml or 60 ml syringe, and the tubes were then sealed and labeled.

### Example 3

### Preparation Of Antiseptic Composition 3 (ASC-3)

Another antiseptic composition including betamethasone valerate as an anti-inflammatory agent (ASC-3) and formulated for topical use was prepared containing the components as listed in the following table:

TABLE 3
Components in ASC-3
Component

SDAG-3 ethanol, 95%, containing 700
mg/100 L Bitrex ™)
o-phenylphenol (OPP)
Benzalkonium chloride (BZK)
Chlorhexidine gluconate (CHG)
Nonoxynol-9
Deionised, double-distilled water
Lemon fragrance No. 431
Betamethasone valerate
Hydroxypropylcellulose

As indicated in Table 3, ASC-3 contained 0.1% betamethasone valerate and was prepared as follows. ASC-1 was prepared as described in Example 1. Approximately 900 ml of ASC-1 was poured into a 1 L glass beaker. Betamethasone valerate USP powder (1 g) was added to the solution and allowed to dissolve with gentle stirring.

In order to formulate the resulting solution as a gel for topical use, hydroxypropylcellulose USP (20 g) was then added slowly to the solution with constant and vigorous stirring. The final concentration of hydroxypropylcellulose USP in the formulation was 2.00%. Once the mixture was gelled the volume of the formulated composition was adjusted to 1000 ml with an additional volume of ASC-1 and mixed for another 1 minute until the whole mixture was homogenous, to obtain ASC-3. Individual plastic tubes were each filled with 15 g of ASC-3 using a 30 ml or 60 ml syringe, and the tubes were then sealed and labeled.

### Example 4

### Preparation of Antiseptic Composition 4 (ASC-4)

Another antiseptic composition including betamethasone valerate as an anti-inflammatory agent (ASC-4) and formulated for topical use was prepared containing the components as listed in the following table:

TABLE 4
Components in ASC-4
Component

SDAG-3 ethanol, 95%, containing 700
mg/100 L Bitrex ™)
o-phenylphenol (OPP)
Benzalkonium chloride (BZK)
Chlorhexidine gluconate (CHG)
Nonoxynol-9
Deionised, double-distilled water
Lemon fragrance No. 431
Betamethasone valerate
Hydroxypropylcellulose

As indicated in Table 4, ASC-3 contained 0.1% betamethasone valerate and was prepared as follows. ASC-1 was prepared as described in Example 1. Approximately 500 ml of ASC-1 was mixed with 500 ml of distilled water in a conical graduate to provide a 0.5×ASC-1 composition. Approximately 900 ml of this composition was poured into a 1 L glass beaker. Betamethasone valerate USP powder (1 g) was then added and allowed to dissolve with gentle stirring.

In order to formulate the resulting composition as a gel for topical use, hydroxypropylcellulose USP (20 g) was then added slowly with constant and vigorous stirring. The final concentration of hydroxypropylcellulose USP in the formulation was 2.00%. Once the mixture was gelled the volume of the formulated composition was adjusted to 1000 ml with an additional volume of 0.5× ASC-1 and mixed for another 1 minute until the whole mixture was homogenous, to obtain ASC-4. Individual plastic tubes were each filled with 15 g of ASC-4 using a 30 ml or 60 ml syringe, and the tubes were then sealed and labeled.

### Example 5

### Ability of ASC-1 to Kill Staphylococcus Aureus, Salmonella Choleraesuis and Pseudomonas Aeruginosa

The following example demonstrates the ability of three preparations of ASC-1 to kill Staphylococcus aureus, Salmonella choleraesuis and Pseudomonas aeruginosa. Testing was carried out according to the AOAC “Official Methods of Analysis, Germicidal Spray Products as Disinfectants, Test Method 961.02” (1990).

### Protocol:

Test Organisms: Staphylococcus aureus (ATCC #6538), Salmonella choleraesuis (ATCC #10708) and Pseudomonas aeruginosa (ATCC #15442).

Test Product: As indicated above, three preparations or lots of ASC-1 were tested: ASC-1a, ASC-1b, and ASC-1c.

Growth & Subculture Media: S. aureus and S. cholerasuis were grown in synthetic broth; P. aeruginosa was grown in nutrient broth. Letheen Broth was the subculture media. The broth culture was supplemented with 0.25 mL of fetal bovine serum for each 4.75 mL of broth culture to yield a 5% soil load.

Carriers & Carrier Contamination: Non-corrosive 18 mm×36 mm glass slides were placed in glass Petri dishes and sterilized in an air oven for 2 hours at approximately 180° C. Using a biological safety hood, individual sterile plastic Petri dishes were matted with 2 pieces of 9 cm filter paper and then a sterile glass slide was transferred into each.

The test cultures were thoroughly mixed and allowed to settle for 10 minutes prior to use and then a 5% soil load, as described above, was added to each culture. Individual glass slides were inoculated with 0.01 mL culture using a calibrated pipettor and the inoculum was uniformly spread over the entire surface of the slide in the Petri dish. The dish was then covered immediately and the procedure repeated for all slides. The slides were allowed to dry for 30 minutes at 36° C.

Test Product: The test product was not diluted prior to use. Three batches of test substance were assayed, as indicated above.

Medication (Exposure): For each batch of the test product, 50 carriers were sprayed individually at staggered intervals for 2 or 3 sprays at a distance of 8-9 inches. Each carrier remained in contact with the germicide for 3 minutes at room temperature (21° C.).

Subculture: Following the spray treatment, the remaining liquid was drained off. Each medicated carrier was then transferred using sterile forceps at identical staggered intervals to 20 mL aliquots of Letheen Broth contained in tubes.

Incubation & Observation: The subculture tubes were incubated for approximately 44-48 hours at 36° C. Following incubation, the medium in each bottle was observed for the presence of visible growth. Subculture broths showing growth were inoculated onto appropriate agar, incubated and the resultant growth characterized in order to confirm or rule out the presence of the test organism.

Neutralization Confirmation: A minimum of 10% of the subculture broths showing no growth were challenged with low levels of the organism to confirm the neutralization of the test substance (Neutralization control). The results of this experiment are shown in Table 6 below.

Carrier Quantification: The numbers of each test organism present on representative unexposed inoculated dried carriers were determined as follows:
1. Inoculated dried carriers were added to neutralizing broth at a ratio of 1 carrier to 10 mL neutralizing formula and the mixture was vortex mixed; the neutralizing formula was produced by adding the following product neutralizers to a liter of Butterfield's Phosphate buffer solution: KH₂PO₄ (0.4 grams), Na₂HPO₄ (10.3 grams), Triton X-100 (1.0 gram), Polysorbate 80 (50.0 grams), Lecithin (200 grams), and Sodium Oleate (6.0 grams), prior to sterilization.
2. Appropriate serial 10-fold dilutions were prepared and spread-plated on TSA containing 5% sheep blood (BAP) and then incubated 24-25 hours at 36° C.; and
3. The colonies were enumerated and the following calculation performed:

[(avg # colonies on plates at dilution used)×(dilution factor)×(volume of neutralizer)] divided by the number of carriers tested
The results of carrier quantification are shown in Table 7 below.

Phenol Resistance Control: The phenol resistance of the test organisms were determined per the AOAC method. The results of this control are depicted in Table 8 following.

Test Controls: The following controls were run to demonstrate study validity:
1. Purity Control: A “streak plate for isolation” was performed on each organism culture and, following incubation, was examined to confirm the presence of a pure culture.
2. Viability Control: The viability of a representative inoculated carrier of each test organism was confirmed by subculture.
3. Organic Soil Sterility Control: Uninoculated media were incubated and observed for lack of growth to confirm sterility.
4. Carrier Sterility Control: A representative sterile carrier was added to the subculture medium, incubated and then observed for lack of growth to confirm sterility.

The results of these controls are shown in Table 9 below

### Results:

The results showing the effect on ASC-1a, ASC-1b and ASC-1c are shown in Table 5 below.

TABLE 5
Evaluation of Growth in Carrier Subculture

Number of Carriers

| Sample | | Sample | | Showing |
|---|---|---|---|---|
| ID | Organism | Dilution | Exposed | Growth |
| ASC-1a | S. aureus | None | 60 | 0 |
| | S. cholerasuis | None | 60 | 0 |
| | P. aeruginosa | None | 60 | 1 |
| ASC-1b | S. aureus | None | 60 | 0 |
| | S. cholerasuis | None | 60 | 0 |
| | P. aeruginosa | None | 60 | 0 |
| ASC-1c | S. aureus | None | 60 | 1 |
| | S. cholerasuis | None | 60 | 0 |
| | P. aeruginosa | None | 60 | 0 |

Showing Growth = Number of carriers showing growth of the test organism.

The subcultures of positive tubes (i.e. tubes showing growth) demonstrated a pure culture of the control organism.

TABLE 6
Neutralization Confirmation Results:

| | | Neutralization Confirmation | | |
|---|---|---|---|---|
| Sample | | Inoculum | # Subculture | Number |
| ID | Organism | CFU/mL | Tubes Tested | Positive |
| ASC-1a | S. aureus | 16 | 6 | 6 |
| | S. cholerasuis | 4 | 6 | 6 |
| | P. aeruginosa | 3 | 6 | 6 |
| ASC-1b | S. aureus | 4 | 6 | 6 |
| | S. cholerasuis | 4 | 6 | 6 |
| | P. aeruginosa | 3 | 6 | 6 |
| ASC-1c | S. aureus | 4 | 6 | 6 |
| | S. cholerasuis | 4 | 6 | 6 |
| | P. aeruginosa | 3 | 6 | 6 |

The neutralization controls showed growth, eliminating bacteriostasis as the cause of lack of growth in the test system.

TABLE 7
Carrier Quantitation Results:

| Test Organism | Result |
|---|---|
| Staphylococcus aureus | 6.1 × 10⁶ CFU/carrier |
| Salmonella cholerasuis | 1.7 × 10⁴ CFU/carrier |
| Pseudomonas aeruginosa | 1.9 × 10⁶ CFU/carrier |

CFU = Colony Forming Unit

TABLE 8
Phenol Resistance of Organism:

| | | Exposure Time | | |
|---|---|---|---|---|
| Organism | Dilution | 5 Minutes | 10 Minutes | 15 Minutes |
| S. aureus | 1:60 | + | + | 0 |
| | 1:70 | + | + | + |
| S. cholerasuis | 1:90 | + | 0 | 0 |
| | 1:100 | + | + | 0 |
| P. aeruginosa | 1:80 | 0 | 0 | 0 |
| | 1:90 | + | + | + |

+ = Growth of organism in subculture bottles.
0 = No growth in subculture bottles.

Final phenol dilutions were made the day of use from stock phenol within manufacturer's stability specification.

TABLE 9
Results of Test Controls:

| | Results | | |
|---|---|---|---|
| Type of Control | S. aureus | S. cholerasuis | P. aeruginosa |
| Purity Control | Pure | Pure | Pure |
| Viability Control | Growth | Growth | Growth |

| | |
|---|---|
| Organic Soil | No Growth |
| Sterility Control | |
| Medium Sterility Control | No Growth |
| Carrier Sterility Control | No Growth |

The results from Table 9 confirmed study validity.

### Analysis & Conclusions:

ASC-1a and ASC-1b did not demonstrate growth of S. aureus in any of the 60 subculture tubes following 3 minute exposure period in the presence of a 5% soil load. ASC-1c demonstrated growth of S. aureus in 1 of the 60 subculture tubes following 3 minute exposure period in the presence of a 5% soil load. ASC-1a, ASC-1b and ASC-1c demonstrated efficacy against Staphylococcus aureus.

ASC-1a, ASC-1b and ASC-1c did not demonstrate growth of S. cholerasuis in any of the 60 subculture tubes following 3 minute exposure period in the presence of a 5% soil load. Under the conditions of this investigation, these preparations of antiseptic compositions demonstrated efficacy against Salmonella cholerasuis.

ASC-1b and ASC-1c did not demonstrate growth of P. aeruginosa in any of the 60 subculture tubes following 3 minute exposure period in the presence of a 5% soil load. ASC-1a demonstrated growth of S. aureus in 1 of the 60 subculture bottles following 3 minute exposure period in the presence of a 5% soil load. Under the conditions of this investigation, ASC-1a, ASC-1b and ASC-1c demonstrated efficacy against Salmonella cholerasuis.

These example indicates that ASC-1 demonstrates efficacy against gram positive and gram negative bacteria.

### Example 6

### Evaluation of the Ability of ASC-1 to Kill Tricophyton Mentagrophytes

The following example demonstrates the ability of the antiseptic: compositions according to the invention to kill fungi of the genus Trichophyton. The ability of two preparations of ASC-1 to kill Trichophyton mentagrophytes was tested according to the AOAC “Official Methods of Analysis, Germicidal Spray Products as Disinfectants, Test Method 961.02” (1990), modified for fungi.

### Protocol:

Test Organisms: Trichophyton mentagrophytes (ATCC #9533).

Test Product: Test substances ASC-1b and ASC-1c were prepared as described in Example 1 used undiluted.

Subculture Media: Sabourauds Dextrose Both containing 0.07% Lecithin and 0.5% Polysorbate (Tween 80).

Soil Load Description: 0.35 mL broth culture +4.75 mL fetal bovine serum (5%).

Preparation of Conidial Suspension: From the stock culture of Trichophyton mentagrophytes, a conidial suspension was prepared by inoculating Sabourand Dextrose agar plates and incubating for 10-15 days at 25-30° C. Following incubation, the mycelia were removed from all plates using a sterile spatula or swab. The mycelia were transferred to a glass screw-cap vessel containing sterile glass beads and 300 mL saline. The mixture was vortex mixed and filtered through sterile gauze to remove hyphal fragments. The conidial concentration was estimated using a hemacytometer. This Stock Spore Suspension was stored for ≦4 weeks at 2-8° C. On the day of testing, 0.2 mL aliquot of Triton X-100 and the solid load previously described were added to a 5.0 mL aliquot of the spore suspension.

Carriers: Non-corrosive 1 mm×1 mm glass slides were placed in glass Petri dishes and sterilized in an air oven for 2 hours at approximately 180° C. Using a biological safety hood, individual sterile plastic Petri dishes were matted with 2 pieces of 9 cm filter paper and then a sterile glass slide was transferred into each.

Carrier Contamination: The glass slides were each inoculated with 0.01 mL conidial suspensions (using a 10 μL pipettor) uniformly spreading the culture over the entire surface of the slide in the Petri dish. The dish was then covered immediately and the procedure repeated for all slides. The slides were allowed to dry for 30 minutes at 36° C.

Test substance Use-Dilution: The test substance was not diluted prior to use. The test substance was in solution as determined by visual observation.

Medication: Each slide carrier was sprayed with the germicidal spray product for 2-3 sprays at a distance of 8-9 inches. Each carrier remained in contact with the test substance for 3 minutes at room temperature (26° C.).

Subculture: Following the spray treatment, the remaining liquid was drained off. Each medicated carrier was then transferred using sterile forceps at staggered intervals to 20 mL aliquots of Sabourands Dextrose Broth with 0.07% Lecithin and 0.5% Tween. Secondary subcultures were not required for this study.

Incubation & Observation: The subculture bottles were incubated for a10 days at 27-28° C. Following incubation, the bottles were observed for the presence or absence of visible growth. Subculture broths showing growth were inoculated onto appropriate agar for confirmation of the test organism.

Neutralization Confirmation: The neutralization of the test substance was confirmed by exposing sterile carriers to the test substance and transferring them to primary subculture tubes containing 20 mL of subculture media. The subculture tubes were challenged with low levels of the organism (Neutralization control), incubated as in test and observed for the presence of growth. The results are shown in Table 11.

Carrier Quantification: The numbers of each test organism present on representative unexposed inoculated dried carriers were determines as follows:
4. Inoculated carriers were added to neutralizing broth at a ratio of 1 carrier to 10 mL neutralizing broth and the mixture was vortex mixed;
5. Appropriate serial 10-fold dilutions were prepared and spread-plated on Potato Dextrose Agar and then incubated for 4 days at 26-27° C.; and
6. The colonies were enumerated and the following calculation performed:

(avg # colonies on plates at dilution used)×(dilution factor)×(volume of [neutralizer)] divided by the number of carriers tested
The results are shown in Table 12

Phenol Resistance Control: The phenol resistance of Trichophyton mentagrophytes was determined per the AOAC method. Final phenol dilutions were made the day of use from stock phenol within manufacturer's stability specification. The results are shown in Table 13.

Various test controls were also carried out to determine study validity. The results of these test controls are shown in Table 14 and confirm study validity.

### Results:

TABLE 10
Effect of antiseptic compositions on growth of T. mentagrophytes

| | | | Number of Carriers | |
|---|---|---|---|---|
| Sample | | Sample | | Showing |
| ID | Organism | Dilution | Exposed | Growth |
| ASC-1c | T. mentagrophytes | none | 10 | 0 |
| ASC-1b | T. mentagrophytes | none | 10 | 0 |

Showing Growth = Number of carriers showing growth of the test organism.

The subcultures of positive broths (i.e. bottles showing growth) demonstrated a pure culture of the control organism.

TABLE 11
Neutralization Confirmation Results:

| | | Neutralization Confirmation | | |
|---|---|---|---|---|
| | | Inoculum | # Subculture | Number |
| Sample ID | Organism | CFU/mL | Tubes Tested | Positive |
| ASC-1c | T. mentagrophytes | 4 | 1 | 1 |
| ASC-1b | T. mentagrophytes | 4 | 1 | 1 |

The neutralization controls showed growth, eliminating bacteriostasis as the cause of lack of growth in the test system.

TABLE 12
Carrier Quantitation Results:

| Test Organism | Result |
|---|---|
| T. mentagrophytes | 4.6 × 10⁴ CFU/carrier |

CFU = Colony Forming Unit

TABLE 13
Phenol Resistance of Organism:

Exposure Time

| Organism | Dilution | 5 Minutes | 10 Minutes |
|---|---|---|---|
| T. mentagrophytes | 1:60 | 0 | 0 |
| | 1:70 | + | + |

+ = Growth of organism in subculture bottles.
0 = No growth in subculture bottles.

TABLE 14
Results of Test Controls:

| | Results |
|---|---|
| Type of Control | T. mentagrophytes |
| Purity Control | Pure |
| Viability Control | Growth |
| Organic Soil Sterility Control | No Growth |
| Medium Sterility Control | No Growth |
| Carrier Sterility Control | No Growth |

### Analysis & Conclusion:

T. mentagrophytes exposed to ASC-1b or ASC-1c did not grow in any of the 10 primary subculture bottles following 3 minute exposure period in the presence of a 5% soil load. Under the conditions of this investigation, ASC-1b and ASC-1c demonstrated efficacy against Trichophyton mentagrophytes.

This example demonstrates that ASC-1 is able to kill fungi.

### Example 7

### Virucidal Efficacy of ASC-1 Against Poliovirus Type 1

The following example demonstrates the virucidal efficacy of an antiseptic composition according to the invention. Two preparations of ASC-1 were tested for their ability to kill Poliovirus Type 1 as follows.

### Test System:

Virus: The Brunhilde strain of Poliovirus Type 1 used for this study was obtained from the American Type Culture Collection (ATCC VR-1000). Stock virus was prepared by collecting the supernatant culture fluid from infected culture cells. The cells were disrupted and cell debris removed by centrifugation at approximately 2000 RPM for 5 minutes at approximately 4° C. The supernatant was removed, aliquoted and the high titer stock virus was stored at ≦−70° C. until the day of use. Then an aliquot of stock virus (ViroMed Lot VML-P34) was removed, thawed and refrigerated until use in the assay. The stock virus culture had fetal bovine serum added to obtain a final organic load concentration of 5% fetal bovine serum. The stock virus demonstrated cytopathic effects (CPE) typical of Poliovirus on Vero cells (African green monkey kidney cells).

Test Cell Cultures: Vero cells were obtained from ViroMed Laboratories, Inc., Cell Culture Division. Cultures were grown and propagated in-house and used as monolayers in disposable tissue culture labware. On the day of testing, cells were observed as having proper cell integrity and, therefore, acceptable for use in the study.

Test Medium: Test medium used in this study was Eagles minimal essential medium (E-MEM) supplemented with 5% heat-inactivated fetal bovine serum (FBS), 10 μg/mL gentamicin, 100 units/mL penicillin and 2.5 μg/mL Fungizone.

Table 15 lists the test and control groups, the dilutions assayed and the numbers of cultures used.

TABLE 15
Number of Dilutions and Cultures for Virucidal Efficacy Study

| | | Cultures | |
|---|---|---|---|
| | Dilutions Assayed | per | Total |
| Test or Control Group | (log₁₀) | Dilution | Cultures |
| Cell Control | N/A | 4 | 4/group |
| Dried Virus Control | −1, −2, −3, −4, −5, −6, −7, −8 | 4 | 32 |
| (Group A) | | | |
| Sample Lot #1 + Virus | −1, −2, −3, −4, −5, −6, −7, −8 | 4 | 32 |
| (Group B) | | | |
| Sample Lot #2 + Virus | −1, −2, −3, −4, −5, −6, −7, −8 | 4 | 32 |
| (Group B) | | | |
| Cytotoxicity of Lot #1 | −1, −2, −3, −4, −5, −6, −7, −8 | 4 | 32 |
| (Group C) | | | |
| Cytotoxicity of Lot #2 | −1, −2, −3, −4, −5, −6, −7, −8 | 4 | 32 |
| (Group C) | | | |
| Non-Virucidal Level - | −1, −2, −3, −4, −5, −6, −7, −8 | 4 | 32 |
| Lot #1 (Group D) | | | |
| Non-Virucidal Level - | −1, −2, −3, −4, −5, −6, −7, −8 | 4 | 32 |
| Lot #2 (Group D) | | | |

### Methods:

1. Preparation of Test Substance: Two preparations or lots of the antiseptic composition ASC-1 were tested, ASC-1b and ASC-1c. These lots were prepared as described in Example 1.
2. Preparation of Virus Films: Films of viruses were prepared by spreading 0.2 mL of virus inoculum uniformly over the bottom of 3 separate 100×15 mm sterile glass Petri dishes. The virus films were air dried at 10° C. in a relative humidity of 50% until visibly dry (25 minutes).
3. Sephadex Gel Filtration: To reduce the cytotoxic level of the virus-antiseptic composition mixture prior to assay and/or to reduce the virucidal level of the antiseptic composition, virus was separated from the antiseptic composition by filtration through Sephadex gel. Columns of Sephadex LH-20-100 were equilibrated with phosphate buffered saline containing 1% albumin, centrifuged for 3 minutes to clear the void volume, loaded with 2.0 mL of virus-antiseptic composition mixture and immediately passed through the column utilizing the syringe plunger.
4. Treatment of Virus Films with Test Substance (Group B, Table 1): For each preparation or lot of antiseptic composition, separate dried virus films were exposed for 3 minutes at room temperature (25° C.) to the amount of spray released under use conditions. The carriers were sprayed with 3 sprays at a distance of 8-9 inches from the surface. The virus films were completely covered with the antiseptic composition. Following the exposure time, the plates were scraped with a cell scraper to resuspend the contents of the plate and the virus-antiseptic composition mixture was immediately passed through a Sephadex column utilizing the syringe plunger in order to detoxify the mixture. The filtrate (10⁻¹ dilution) was then titered by serial dilution and assayed for infectivity.
5. Treatment of Virus Control Film (Group A, Table 16): A virus film was prepared as previously described (see paragraph 2). The control film was exposed to 2.0 mL test medium for the same amount of time as the test film was exposed to the antiseptic composition. The virus was then scraped and passed through a Sephadex column in the same manner as the test virus (see paragraph 4).
6. Cytotoxicity Assay (Group C, Table 17): An aliquot of each lot of test substance was sprayed onto separate sterile Petri dishes, scraped, Sephadex filtered, diluted serially in medium and inoculated into Vero cell cultures. Cytotoxicity of the Vero cell cultures was scored at the same time as virus-antiseptic composition and virus control cultures.
7. Assay of Non-Virucidal Level of Test Substance (Group D, Table 18): Each dilution of the Sephadex-filtered antiseptic composition (antiseptic composition control for cytotoxicity assay) was mixed with an aliquot of low titer stock virus and the resulting mixtures of dilutions were assayed for infectivity in order to determine the dilution(s) of antiseptic composition at which virucidal activity, if any, was retained. Dilutions that showed virucidal activity were not considered in determining the reduction in infectivity by the test substance.
8. Infectivity Assays: The Vero cell line, which exhibits CPE in the presence of Poliovirus Type 1, was used as the indicator cell line in the infectivity assays. Cells in multiwell culture dishes were inoculated in quadruplicate with 0.1 mL of the dilutions prepared from test and control groups. Uninfected indicator cell cultures (cell controls) were inoculated with test medium alone. Cultures were incubated at 37.3° C. in a humidified atmosphere of 6.0-6.1% CO₂ in sterile disposable cell culture labware. The cultures were scored periodically for seven days for the absence or presence of CPE, cyotoxicity and viability.

CALCULATION OF TITERS: Viral and cytotoxicity titers are expressed as −log₁₀ of the 50% titration endpoint for infectivity (TCID₅₀) or cytotoxicity (TCD₅₀) respectively, as calculated by the method of Spearman Karber:

$$\frac{( Sum of \% mortality at each dilution )}{100} - 0.5 \times ( logarithm of dilution )$$

ANALYSIS AND CONCLUSIONS: Results of tests with ASC-1b and ASC-1c exposed to Poliovirus Type 1 for 3 minutes are shown in Tables 15-17 below. The titer of virus control was 5.75 log₁₀. Following exposure, test virus infectivity was not detected in the virus-test substance mixture for either lot at any dilution tested (≦1.5 log₁₀). Test substance cytotoxicity was observed at 1.5 log₁₀ for ASC-1 b. Test substance cytotoxicity was not observed in the cytotoxicity control at any dilution tests (≦0.5 log₁₀) for ASC-1c. The neutralization control (non-virucidal level of the test substance) indicates that the test substance was neutralized at ≦1.5 log₁₀ for Lot ASC-1b and ≦0.5 log₁₀ for ASC-1c. Taking the cytotoxicity and neutralization control results into consideration, the reduction in virus titer was ≧4.25 log₁₀ for both lots. Under these test conditions, both lots of ASC-1 demonstrated complete inactivation of the Poliovirus Type 1.

TABLE 16
Effects of ASC-1b and ASC-1c Following a 3 Minute Exposure to
Poliovirus Type 1

| | Dried | Poliovirus Type 1 + | Poliovirus Type 1 + |
|---|---|---|---|
| | Virus Control | ASC-1c | ASC-1b |
| Dilution | (GROUP A) | (GROUP B) | (GROUP B) |
| Cell Control | 0000 | 0000 | 0000 |
| 10⁻¹ | ++++ | TTTT | TTTT |
| 10⁻² | ++++ | 0000 | 0000 |
| 10⁻³ | ++++ | 0000 | 0000 |
| 10⁻⁴ | ++++ | 0000 | 0000 |
| 10⁻⁵ | ++++ | 0000 | 0000 |
| 10⁻⁶ | 000+ | 0000 | 0000 |
| 10⁻⁷ | 0000 | 0000 | 0000 |
| 10⁻⁸ | 0000 | 0000 | 0000 |
| TCID₅₀/ | 10^{5.75} | ≦10^{1.5} | ≦10^{1.5} |
| 0.1 mL | | | |

TABLE 17
Cytotoxicity of ASC-1b and ASC-1c on Vero Cell Cultures.

| | Cytotoxicity Control | Cytotoxicity Control |
|---|---|---|
| | ASC-1c | ASC-1b |
| Dilution | (GROUP C) | (GROUP C) |
| Cell Control | 0000 | 0000 |
| 10⁻¹ | 0000 | TTTT |
| 10⁻² | 0000 | 0000 |
| 10⁻³ | 0000 | 0000 |
| 10⁻⁴ | 0000 | 0000 |
| 10⁻⁵ | 0000 | 0000 |
| 10⁻⁶ | 0000 | 0000 |
| 10⁻⁷ | 0000 | 0000 |
| 10⁻⁸ | 0000 | 0000 |
| TCD₅₀/0.1 mL | ≦10^{0.5} | ≦10^{1.5} |

TABLE 18
Non-virucidal Level of Test Substance (Neutralization Control)

| | Virus Control + | Virus Control + |
|---|---|---|
| | Cytotoxicity Control | Cytotoxicity Control |
| | ASC-1c | ASC-1b |
| Dilution | (GROUP D) | (GROUP D) |
| Cell Control | 0000 | 0000 |
| 10⁻¹ | ++++ | TTTT |
| 10⁻² | ++++ | ++++ |
| 10⁻³ | ++++ | ++++ |
| 10⁻⁴ | ++++ | ++++ |
| 10⁻⁵ | ++++ | ++++ |
| 10⁻⁶ | ++++ | ++++ |
| 10⁻⁷ | ++++ | ++++ |
| 10⁻⁸ | ++++ | ++++ |

+ = Positive for the presence of test virus.
0 = No test virus recovered and/or no cytotoxicity present.
T = Cytotoxicity present.

Results of the non-virucidal level control indicate that both preparations of the test substance were neutralized at TCID₅₀ of ≦1.5 log₁₀ for ASC-1b and ≦0.5 log₁₀ for ASC-1c. The results provided in this example indicate that ASC-1 demonstrates efficacy against viruses.

### Example 8

### Evaluation of Virucidal Efficacy of ASC-1 Against Human Immunodeficiency Virus Type 1

The following example demonstrates the virucidal effect of the antiseptic composition according to the present invention. The efficacy of two preparations of ASC-1 against Human Immunodeficiency Virus Type 1 was evaluated as follows.

### Test System:

Virus: The HTLV-IIIB strain of Human Immunodeficiency Virus Type 1 (HIV-1) used for this study was obtained from the Advanced Biotechnologies, Inc., Columbia, Md. Stock virus was prepared by collecting the supernatant culture fluid from infected culture cells as determined by an indirect immunofluorescence assay specific for the HIV-1 antigen. The cells were disrupted and cell debris removed by centrifugation at approximately 1200 RPM for 10 minutes. The supernatant was removed and the virus was concentrated by ultra-centrifugation. The supernatant was removed and the virus pellet was resuspended in test medium, aliquoted and stored at ≦−60° C. until the day of use. Then an aliquot of stock virus (ViroMed Lot HT-111B-2A) was removed, thawed and refrigerated until use in the assay. The stock virus culture was diluted in serum free RPMI-1640 to a final organic load concentration of 5% fetal bovine serum. The stock virus demonstrated cytopathic effects (CPE) typical of HIV on MT-2 cells.

Test Cell Cultures: MT-2 cells (human CD4+ lymphocytes) were obtained from the National Cancer Institute, Frederick, Md. Cultures were grown and propagated in-house and used in suspension in disposable tissue culture labware. On the day of testing, cells were observed as having proper cell integrity and, therefore, acceptable for use in the study.

Test Medium: Test medium used in this study was RPMI 1640 supplemented with 15% heat-inactivated fetal bovine serum (FBS). The medium was also supplemented with 2 mM L-glutamine and 50 μg/mL gentamicin.

Table 19 lists the test and control groups, the dilutions assayed and the numbers of cultures used.

TABLE 19
Number of Dilutions and Cultures for Virucidal Efficacy Study

| | | Cultures | |
|---|---|---|---|
| | Dilutions Assayed | Per | Total |
| Test or Control Group | (log10) | Dilution | Cultures |
| Cell Control | N/A | 4 | 4/group |
| Dried Virus Control | −1, −2, −3, −4, −5, −6, −7 | 4 | 28 |
| (Group A) | | | |
| ASC-1c + Virus | −1, −2, −3, −4, −5, −6, −7 | 4 | 28 |
| (Group B) | | | |
| ASC-1b + Virus | −1, −2, −3, −4, −5, −6, −7 | 4 | 28 |
| (Group B) | | | |
| Cytotoxicity of ASC-1c | −1, −2, −3, −4, −5, −6, −7 | 4 | 28 |
| (Group C) | | | |
| Cytotoxicity of ASC-1b | −1, −2, −3, −4, −5, −6, −7 | 4 | 28 |
| (Group C) | | | |
| Non-Virucidal Level - | −1, −2, −3, −4, −5, −6, −7 | 4 | 28 |
| ASC-1c (Group D) | | | |
| Non-Virucidal Level - | −1, −2, −3, −4, −5, −6, −7 | 4 | 28 |
| ASC-1b (Group D) | | | |

### Methods:

Preparation of Test Substance: Two lots of test substance ASC-1 (ASC-1b and ASC-1c) were prepared as described in Example 1.

Preparation of Virus Films: Films of viruses were prepared by spreading 0.2 mL of virus incoculum uniformly over the bottom of 3 separate 100×15 mm sterile glass Petri dishes. The virus films were air dried at room temperature (22° C.) and then incubated at 37° C. for an additional 30 minutes to increase the level of dryness.

Sephadex Gel Filtration: To reduce the cytotoxic level of the virus-test substance mixture prior to assay and/or to reduce the virucidal level of the test substance, virus was separated from the test substance by filtration through Sephadex gel. Columns of Sephadex LH-20-100 were equilibrated with phosphate buffered saline containing 1% albumin, centrifuged for 3 minutes to clear the void volume, loaded with 2.0 mL of virus-test substance mixture and immediately passed through the column utilizing the syringe plunger.

Treatment of Virus Films with Test Substance (Group B, Table 20): For each lot of test substance, separate dried virus films were exposed for 3 minutes at room temperature (22° C.) to the amount of spray released under use conditions. The carriers were sprayed with 3 sprays at a distance of 8-9 inches from the surface. The virus films were completely covered with the test substance. Following the exposure time, the plates were scraped with a cell scraper to resuspend the contents of the plate and the virus-test substance mixture was immediately passed through a Sephadex column utilizing the syringe plunger in order to detoxify the mixture. The filtrate (10-1 dilution) was then tittered by serial dilution and assayed for infectivity.

Treatment of Virus Control Film (Group A, Table 20): A virus film was prepared as previously described (paragraph 2). The control film was exposed to 2.0 mL test medium for the same amount of time as the test film was exposed to the disinfectant. The virus was then scraped and passed through a Sephadex column in the same manner as the test virus (paragraph 4).

Cytotoxicity Assay (Group C, Table 21): An aliquot of each lot of test substance was sprayed onto separate sterile Petri dishes, scraped, Sephadex filtered, diluted serially in medium and inoculated into MT-2 cell cultures. Cytotoxicity of the MT-2 cell cultures was scored at the same time as virus-test substance and virus control cultures.

Assay of Non-Virucidal Level of Test Substance (Group D, Table 22): Each dilution of the Sephadex-filtered test substance (test substance control for cytotoxicity assay) was mixed with an aliquot of low titer stock virus and the resulting mixtures of dilutions were assayed for infectivity in order to determine the dilution(s) of disinfectant at which virucidal activity, if any, was retained. Dilutions that showed virucidal activity were not considered in determining the reduction in infectivity by the test substance.

Infectivity Assays: The MT-2 cell line, which exhibits CPE in the presence of HIV-1, was used as the indicator cell line in the infectivity assays. Cells in multiwell culture dishes were inoculated in quadruplicate with 0.2 mL of the dilutions prepared from test and control groups. Uninfected indicator cell cultures (cell controls) were inoculated with test medium alone. Cultures were incubated at 37° C. in a humidified atmosphere of 6.0-6.5% CO2 in sterile disposable cell culture labware. The cultures were scored periodically for seven days for the absence or presence of CPE, cyotoxicity and viability.

### Statistical Methods: N/A.

CALCULATION OF TITERS: Viral and cytotoxicity titers are expressed as −log₁₀ of the 50% titration endpoint for infectivity (TCID⁵⁰) or cytotoxicity (TCD₅₀) respectively, as calculated by the method of Spearman Karber:

$$\frac{( Sum of \% mortality at each dilution )}{100} - 0.5 \times ( logarithm of dilution )$$

ANALYSIS AND CONCLUSIONS: Results of test with ASC-1b and ASC-1c exposed to HIV-1 for 3 minutes are shown in Tables 20-22 below. The titer of virus control was 5.25 log₁₀. Following exposure, test virus infectivity was not detected in the virus-test substance mixture for either lot at any dilution tested (≦1.5 log₁₀). Test substance cytotoxicity was observed for both ASC-1b and ASC-1c at 1.5 log₁₀. The neutralization control (non-virucidal level of the test substance) indicates that the test substance was neutralized at ≦1.5 log 10 for both ASC-1b and ASC-1c. Taking the cytotoxicity and neutralization control results into consideration, the reduction in virus titer was ≧3.75 log₁₀ for both ASC-1b and ASC-1c. Under these test conditions, both lots of ASC-1 demonstrated completed inactivation of the HIV-1.

This example indicates that ASC-1 is able to inactivate viruses including HIV-1.

TABLE 20
Effects of ASC-1b and ASC-1c Following a 3 Minute Exposure to
HIV-1

| | Dried Virus | HIV-1 + | HIV-1 + |
|---|---|---|---|
| | Control | ASC-1c | ASC-1b |
| Dilution | (GROUP A) | (GROUP B) | (GROUP B) |
| Cell Control | 0000 | 0000 | 0000 |
| 10-1 | ++++ | TTTT | TTTT |
| 10-2 | ++++ | 0000 | 0000 |
| 10-3 | ++++ | 0000 | 0000 |
| 10-4 | ++++ | 0000 | 0000 |
| 10-5 | +00+ | 0000 | 0000 |
| 10-6 | 000+ | 0000 | 0000 |
| 10-7 | 0000 | 0000 | 0000 |
| TCID₅₀/ | 10^{5.25} | ≦10^{1.5} | ≦10^{1.5} |
| 0.2 ml | | | |

TABLE 21
Cytotoxicity of ASC-1b and ASC-1c on MT-2 Cell Cultures.

| | Cytotoxicity | Cytotoxicity |
|---|---|---|
| | Lot No. 25F902 | Lot No. 17L701 |
| Dilution | (GROUP C) | (GROUP C) |
| Cell Control | 0000 | 0000 |
| 10-1 | TTTT | TTTT |
| 10-2 | 0000 | 0000 |
| 10-3 | 0000 | 0000 |
| 10-4 | 0000 | 0000 |
| 10-5 | 0000 | 0000 |
| 10-6 | 0000 | 0000 |
| 10-7 | 0000 | 0000 |
| TCD⁵⁰/0.2 mL | ≦10^{1.5} | ≦10^{1.5} |

TABLE 22
Non-virucidal Level of Test Substance (Neutralization Control)

| | Virus Control + | Virus Control + |
|---|---|---|
| | Cytotoxicity Control | Cytotoxicity Control |
| | ASC-1c | ASC-1b |
| Dilution | (GROUP D) | (GROUP D) |
| Cell Control | 0000 | 0000 |
| 10-1 | TTTT | TTTT |
| 10-2 | ++++ | ++++ |
| 10-3 | ++++ | ++++ |
| 10-4 | ++++ | ++++ |
| 10-5 | ++++ | ++++ |
| 10-6 | ++++ | ++++ |
| 10-7 | ++++ | ++++ |

+ = Positive for the presence of test virus.
= No test virus recovered and/or no cytotoxicity present.
T = Cytotoxicity present.

### Example 9

### Evaluation of Virucidal Efficacy of ASC-1 Against Herpes Simplex Virus Type 1

SUMMARY: This example demonstrates the ability of an antiseptic composition according to the invention to inactivate viruses of the Herpes genus. The ability of two preparations or lots of ASC-1 (ASC-1b and ASC-1c) to kill Herpes Simplex Virus Type 1 (HSV-1) was tested as follows.

### Test System:

Virus: The F(1) strain of Herpes Simplex Virus Type 1 used for this study was obtained from the American Type Culture Collection (ATCC VR-733). Stock virus was prepared by collecting the supernatant culture fluid from infected culture cells. The cells were disrupted and cell debris removed by centrifugation at approximately 2000 RPM for 5 minutes at approximately 4° C. The supernatant was removed, aliquoted and the high titer stock virus was stored at ≦−70° C. until the day of use. Then an aliquot of stock virus (ViroMed Lot VML-H41) was removed, thawed and refrigerated until use in the assay. The stock virus culture had fetal bovine serum added to obtain a final organic load concentration of 5% fetal bovine serum. The stock virus demonstrated cytopathic effects (CPE) typical of HSV-1 on rabbit kidney cells.

Test Cell Cultures: Rabbit Kidney (RK) cells were obtained from ViroMed Laboratories, Inc., Cell Culture Division. Cultures were grown and propagated in-house and used as monolayers in disposable tissue culture labware. On the day of testing, cells were observed as having proper cell integrity and, therefore, acceptable for use in the study.

Test Medium: Test medium used in this study was Eagles minimal essential medium (E-MEM) supplemented with 10% heat-inactivated fetal bovine serum (FBS), 10 μg/mL gentamicin, 100 units/mL penicillin and 2.5 μg/mL Fungizone.

Table 23 lists the test and control groups, the dilutions assayed and the numbers of cultures used.

TABLE 23
Number of Dilutions and Cultures for Virucidal Efficacy Study

| | | Cultures | |
|---|---|---|---|
| | Dilutions Assayed | per | Total |
| Test or Control Group | (log₁₀) | Dilution | Cultures |
| Cell Control | N/A | 4 | 4/group |
| Dried Virus Control | −1, −2, −3, −4, −5, −6, −7, | 4 | 36 |
| (Group A) | −8, −9 | | |
| Sample ASC-1c + Virus | −1, −2, −3, −4, −5, −6, −7, | 4 | 36 |
| (Group B) | −8, −9 | | |
| Sample ASC-1b + Virus | −1, −2, −3, −4, −5, −6, −7, | 4 | 36 |
| (Group B) | −8, −9 | | |
| Cytotoxicity of ASC-1c | −1, −2, −3, −4, −5, −6, −7, | 4 | 36 |
| (Group C) | −8, −9 | | |
| Cytotoxicity of ASC-1b | −1, −2, −3, −4, −5, −6, −7, | 4 | 36 |
| (Group C) | −8, −9 | | |
| Non-Virucidal Level - | −1, −2, −3, −4, −5, −6, −7, | 4 | 36 |
| ASC-1c (Group D) | −8, −9 | | |
| Non-Virucidal Level - | −1, −2, −3, −4, −5, −6, −7, | 4 | 36 |
| ASC-1b (Group D) | −8, −9 | | |

### Methods:

9. Preparation of Test Substance: Two lots or preparations of ASC-1 (ASC-1b and ASC-1c) were prepared as described in Example 1.
10. Preparation of Virus Films: Films of viruses were prepared by spreading 0.2 mL of virus incoculum uniformly over the bottom of 3 separate 100×15 mm sterile glass Petri dishes. The virus films were air dried at 10° C. in a relative humidity of 53% until visibly dry (30 minutes).
11. Sephadex Gel Filtration: To reduce the cytotoxic level of the virus-test substance mixture prior to assay and/or to reduce the virucidal level of the test substance, virus was separated from the test substance by filtration through Sephadex gel. Columns of Sephadex LH-20-100 were equilibrated with phosphate buffered saline containing 1% albumin, centrifuged for 3 minutes to clear the void volume, loaded with 2.0 mL of virus-test substance mixture and immediately passed through the column utilizing the syringe plunger.
12. Treatment of Virus Films with Test Substance (Group B, Table 24): For each lot of test substance, separate dried virus films were exposed for 3 minutes at room temperature (25° C.) to the amount of spray released under use conditions. The carriers were sprayed with 3 sprays at a distance of 8-9 inches from the surface. The virus films were completely covered with the test substance. Following the exposure time, the plates were scraped with a cell scraper to resuspend the contents of the plate and the virus-test substance mixture was immediately passed through a Sephadex column utilizing the syringe plunger in order to detoxify the mixture. The filtrate (10⁻¹ dilution) was then tittered by serial dilution and assayed for infectivity.
13. Treatment of Virus Control Film (Group A, Table 24): A virus film was prepared as previously described (see paragraph 2). The control film was exposed to 2.0 mL test medium for the same amount of time as the test film was exposed to the test substance. The virus was then scraped and passed through a Sephadex column in the same manner as the test virus (see paragraph 4).
14. Cytotoxicity Assay (Group C, Table 25): An aliquot of each lot of test substance was sprayed onto separate sterile Petri dishes, scraped, Sephadex filtered, diluted serially in medium and inoculated into RK cell cultures. Cytotoxicity of the RK cell cultures was scored at the same time as virus-test substance and virus control cultures.
15. Assay of Non-Virucidal Level of Test Substance (Group D, Table 261: Each dilution of the Sephadex-filtered test substance (test substance control for cytotoxicity assay) was mixed with an aliquot of low titer stock virus and the resulting mixtures of dilutions were assayed for infectivity in order to determine the dilution(s) of test substance at which virucidal activity, if any, was retained. Dilutions that showed virucidal activity were not considered in determining the reduction in infectivity by the test substance.
16. Infectivity Assays: The RK cell line, which exhibits CPE in the presence of HSV-1, was used as the indicator cell line in the infectivity assays. Cells in multiwell culture dishes were inoculated in quadruplicate with 0.1 mL of the dilutions prepared from test and control groups. Uninfected indicator cell cultures (cell controls) were inoculated with test medium alone. Cultures were incubated at 37.3° C. in a humidified atmosphere of 6.0-6.1% CO₂ in sterile disposable cell culture labware. The cultures were scored periodically for seven days for the absence or presence of CPE, cyotoxicity and viability.

CALCULATION OF TITERS: Viral and cytotoxicity titers are expressed as −log₁₀ of the 50% titration endpoint for infectivity (TCID₅₀) or cytotoxicity (TCD₅₀) respectively, as calculated by the method of Spearman Karber:

$$\frac{( Sum of \% mortality at each dilution )}{100} - 0.5 \times ( logarithm of dilution )$$

ANALYSIS AND CONCLUSIONS: Results of test with ASC-1b and ASC-1c exposed to HSV-1 for 3 minutes are shown in Tables 24-26 below. The titer of virus control was 6.75 log₁₀. Following exposure, test virus infectivity was not detected in the virus-test substance mixture for either lot at any dilution tested (≦1.5 log₁₀ for ASC-1c and ≦0.5 log₁₀ for ASC-1b). Test substance cytotoxicity was not observed at any dilution used tested (≦0.5 log₁₀) for ASC-1c. However, test substance cytotoxicity was observed in the cytotoxicity control for ASC-1b at 1.5 log₁₀. The neutralization control (non-virucidal level of the test substance) indicates that the test substance was neutralized at ≦0.5 log₁₀ for ASC-1c and ≦1.5 log₁₀ for ASC-1b. Taking the cytotoxicity and neutralization control results into consideration, the reduction in virus titer was ≧5.25 log₁₀ for both lots of ASC-1.

These results indicate that both lots of ASC-1 demonstrated completed inactivation of HSV-1.

TABLE 24
Effects of ASC-1c and ASC-1b Following a 3 Minute Exposure to
HSV-1

| | Dried Virus | Poliovirus Type 1 + | Poliovirus Type 1 + |
|---|---|---|---|
| | Control | Lot No. 25F902 | Lot No. 17L701 |
| Dilution | (GROUP A) | (GROUP B) | (GROUP B) |
| Cell Control | 0000 | 0000 | 0000 |
| 10⁻¹ | ++++ | TTTT | 0000 |
| 10⁻² | ++++ | 0000 | 0000 |
| 10⁻³ | ++++ | 0000 | 0000 |
| 10⁻⁴ | ++++ | 0000 | 0000 |
| 10⁻⁵ | ++++ | 0000 | 0000 |
| 10⁻⁶ | ++++ | 0000 | 0000 |
| 10⁻⁷ | 000+ | 0000 | 0000 |
| 10⁻⁸ | 0000 | 0000 | 0000 |
| 10⁻⁹ | 0000 | 0000 | 0000 |
| TCID₅₀/ | 10^{6.75} | ≦10^{1.5} | ≦10^{0.5} |
| 0.1 mL | | | |

TABLE 25
Cytotoxicity of ASC-1c and ASC-1b on Rabbit Kidney (RK) Cell
Cultures.

| | Cytotoxicity Control | Cytotoxicity Control |
|---|---|---|
| | Lot No. 25F902 | Lot No. 17L701 |
| Dilution | (GROUP C) | (GROUP C) |
| Cell Control | 0000 | 0000 |
| 10⁻¹ | 0000 | TTTT |
| 10⁻² | 0000 | 0000 |
| 10⁻³ | 0000 | 0000 |
| 10⁻⁴ | 0000 | 0000 |
| 10⁻⁵ | 0000 | 0000 |
| 10⁻⁶ | 0000 | 0000 |
| 10⁻⁷ | 0000 | 0000 |
| 10⁻⁸ | 0000 | 0000 |
| 10⁻⁹ | 0000 | 0000 |
| TCD₅₀/0.1 mL | ≦10^{0.5} | 10^{1.5} |

TABLE 26
Non-virucidal Level of Test Substance (Neutralization Control)

| | Virus Control + | Virus Control + |
|---|---|---|
| | Cytotoxicity Control | Cytotoxicity Control |
| | Lot No. 25F902 | Lot No. 17L701 |
| Dilution | (GROUP D) | (GROUP D) |
| Cell Control | 0000 | 0000 |
| 10⁻¹ | ++++ | TTTT |
| 10⁻² | ++++ | ++++ |
| 10⁻³ | ++++ | ++++ |
| 10⁻⁴ | ++++ | ++++ |
| 10⁻⁵ | ++++ | ++++ |
| 10⁻⁶ | ++++ | ++++ |
| 10⁻⁷ | ++++ | ++++ |
| 10⁻⁸ | ++++ | ++++ |
| 10⁻⁹ | ++++ | ++++ |

+ = Positive for the presence of test virus.
1 = No test virus recovered and/or no cytotoxicity present.
T = Cytotoxicity present.

### Example 10

### Evaluation of Antimycobacterial Activity of ASC-1 Against Mycobacterium Tuberculosis

The following example demonstrates the ability of antiseptic compositions according to the invention to kill bacteria of the genus Mycobacterium. ASC-1 was prepared as described in Example 1 and was tested using the following protocol.

Methods: 55×13 mm diameter sterile coverslips were coated with approximately 10⁷ Mycobacterium tuberculosis strain Erdman ATCC #35801. This was achieved by adding 10 μl of a suspension of mycobacteria at 10⁹/ml to the coverslip, spreading evenly with the tip of the applicator and allowing to air dry. A positive control group (Group 1) to assess the number of viable bacteria on the coverslip was processed by placing 5 coated coverslips into 500 μl broth, sonicating for 10 seconds with a probe sonicator to disperse the bacteria and plating serial dilutions onto agar growth media (Middlebrook 71-110). Ten test samples were prepared by coating coverslips by spraying samples of ASC-1 3 times from a distance of 12 inches. The coverslips were then drained after either 5 minutes (5 coverslips, Group 2) or 10 minutes (5 coverslips, Group 3) using Whatman™ filter paper and placed in 500 μl broth and processed as described above for the controls. Additional controls were also prepared as follows. 5 coverslips coated with bacteria were sprayed with 1% Septol, a known disinfectant, left for 5 minutes and processed as above (Group 4). Another 5 coverslips were coated with bacteria, sprayed with 70% ethanol, also a known disinfectant, left for 5 minutes and processed as above (Group 5). A negative control group (Group 6) was also prepared using 5 coverslips with no bacterial inoculation but otherwise processed as above.

Results: The control treatments (Septol and ethanol) were applied in the same way as the ASC-1 samples. Approximately 90% of the ASC-1 was observed to dry on the coverslip after approximately 6 minutes. A residual milky white liquid remained at 10 minutes. This is similar to 70% ethanol which is mostly dry after 6 minutes with a residual amount of water remaining at 10 minutes.

The results are shown in Table 27.

TABLE 27
Effect of ASC-1 on Mycobacterium tuberculosis

| Experimental Groups | Log₁₀¹ | Log₁₀ | Percent |
|---|---|---|---|
| Group 1: Positive Control | 7.35 ± 0.10 | Not | Not applicable |
| | | applicable | |
| Group 2: ASC-1 (5 min)² | | | |
| Mean of all 20 samples. | 0.89 ± 1.07 | 6.46 | 99.99997 |
| Mean of the 9 positive | 1.97 ± 0.55 | 5.38 | 99.99958 |
| samples | | | |
| Group 3: ASC-1 (10 min)³ | | | |
| Mean of all 20 samples. | 0.76 ± 0.89 | 6.59 | 99.99998 |
| Mean of the 10 positive | 1.52 ± 0.61 | 5.83 | 99.99985 |
| samples | | | |
| Group 4: 1% Septol (10 min) | 5.92 ± 0.64 | 1.43 | 96.29 |
| Group 5: 70% Ethanol | 0.0 | 7.35 | 100 |
| (10 min) | | | |
| Group 6: Negative Control | 0.0 | Not | Not |
| | | applicable | applicable |

### Example 11

### Evaluation of Bactericidal Activity of ASC-1 Against 8 Bacterial Species

SUMMARY: This example demonstrates the ability of an antiseptic composition according to the invention to kill several genera of bacteria. The ability of a preparation of ASC-1 to kill 8 different bacterial species was tested as described below using a modification of the methods described in the Draft European Standard, prEN 12054, “Chemical Disinfectants and Antispetics—Products for Hygienic and Surgical Handrub and Handwash—Bactericidal Activity—Test Method and Requirements (1995).”

### Methodology:

Test Product: A preparation of ASC-1, ASC-1 g, was prepared as described in Example 1, and used without dilution as the product test solution (i.e., full-strength).

Bacterial strains: The bacterial strains evaluated were American Type Culture Collection (ATCC) and National Collection of Type Cultures (NCTC) Strains of Enterococcus faecium (ATCC #6057), Enterococcus faecium VRE MDR (ATCC #51559), Enterococcus hirae (ATCC #10541), Escherichia coli K12 (ATCC #10538), Proteus mirabilis (ATCC #14153), Pseudomonas aeruginosa (ATCC #15442), Staphylococcus aureus aureus (ATCC #6358) and Staphylococcus aureus MRSA (Clinical Isolate; BSLI #051707MRSa2).

Inoculum Preparation: Approximately 48 hours prior to testing, inocula from each species were suspended in Tryptone Sodium Chloride (TSC) and inoculated onto the surface of Tryptic Soy. Agar (TSA) in Petri plates and incubated at 35°±2° C. for 18 to 24 hours. A suspension of each species was prepared 18-24 hours prior to testing by rinsing cultures from the solid media with sterile TSC. The purity of each suspension was verified by streaking for isolation on TSA. Aliquots of each suspension were spread-plate onto the surface of additional plates of TSA and incubated at 35°±2° C. for 18 to 24 hours, or until sufficient growth was observed. This produced lawns of the bacteria on the surface of the agar plates and colonies from these plates were used to prepare the bacterial test suspensions.

Bacterial Test Suspensions: Immediately prior to initiating the Bacterial Assay Procedure and the Dilution-Neutralization Validation Procedure, a suspension of each bacterial species was prepared in sterile TSC by suspending colonies from the solid media previously prepared to achieve suspension concentrations of approximately 1×10⁹ CFU/mL as determined visually based on comparison to a McFarland Standard. These suspensions were diluted, as necessary, with additional TSC to produce concentrations of approximately 1×10⁸ to 3×10⁸ CFU/mL for use in the Bacterial Assay Procedure and 1×10³ to 3×10³ CFU/mL for use in the Dilution-Neutralization Validation Procedure. These test suspensions were maintained in a water bath at 20°±1° C. and used within 2 hours of preparation.

BACTERICIDAL ASSAY PROCEDURES: Prior to testing, the test product (ASC-1 g), bacterial test suspensions and all diluting fluids were placed in a water bath at 20°±1° C. and allowed to equilibrate for no less than 15 minutes, or until the temperature of the solutions had stabilized at 20°±1° C.

Initial Population Determination (Bacterial Test Suspension Controls): A 1.0 mL, aliquot of the bacterial test suspension containing approximately 1×10⁸ to 3×10⁸ CFU/mL was transferred into a sterile test tube containing 9.0 mL of TSC and mixed thoroughly. Ten-fold dilutions (i.e. 10⁻¹, 10⁻², 10⁻³, 10⁻⁴, 10⁻⁵ and 10⁻⁶) of each suspension were prepared in TSC. Using Tryptic Soy Agar with product neutralizers as identified in Example 5. (TSA+), pour-plates were prepared in duplicate for each suspension by plating 1.0 mL aliquots of the final dilutions (i.e. 10⁻⁴, 10⁻⁵ and 10⁻⁶). The plates were incubated at 35°±2° C. for 42-28 hours, or until sufficient growth was observed.

Testing Procedure—Test “D”: A 1.0 mL aliquot of the bacterial test suspension containing approximately 1×10⁸ to 3×10⁸ CFU/mL was transferred into a sterile test tube containing 9.0 mL of the test product and mixed thoroughly. The inoculated tube was then replaced in the water bath. Each challenge strain was exposed to the test product at 20°±1° C. for 30 seconds, and 1, 3, 5, 10, 15 and 30 minutes, timed using a calibrated minute/second timer. After each exposure time had lapsed, 1.0 mL aliquots were removed from each tube containing test product/bacterial test suspension and placed in separate sterile tubes containing 8.0 mL of Neutralizing Formulation (NF, as described in Example 5) and 1.0 mL of sterile Water-for-Irrigation, USP (WFI) and mixed thoroughly with a vortex mixer. Ten-fold dilutions (i.e. 10⁻², 10⁻³, 10⁻⁴, and 10⁻⁵) of the suspensions of each bacterial species were immediately prepared in NF, mixing thoroughly using a vortex mixer between dilutions. Then 1.0 mL aliquots of each dilution were pour-plated in duplicate, using TSA+, producing plated dilutions of 10⁻¹, 10⁻², 10⁻³, 10⁻⁴, and 10⁻⁵. These plates were incubated at 35°±2° C. for 42-48 hours or until sufficient growth was observed.

Data Collection: Following incubation, the colonies on the plates were counted manually using a hand-tally counter. Counts in the range of 30 to 300 CFU were used preferentially in the data calculations.

NEUTRALIZER VALIDATION PROCEDURE (DILUTION-NEUTRALIZATION METHOD): Bacterial test suspensions of each challenge species containing approximately 1×10³ to 3×10³ CFU/mL were prepared as described in the Bacterial Test Suspension section above. Prior to testing, the test product, the bacterial test suspensions and all diluting fluids were placed in a water bath at 20°±1° C. and allowed to equilibrate for no less than 15 minutes, or until the temperature of the solutions had stabilized at 20°±1° C.

Initial Populations Determination—Bacterial Test Suspensions (N)— Test “C”: A 1.0 mL aliquot of the bacterial test suspension containing approximately 1×10³ to 3×10³ CFU/mL was transferred into a sterile tube containing 9.0 mL of TSC and mixed thoroughly. Then 1.0 mL aliquots of this suspension were pour-plated, in duplicate, using TSA+. The plates were incubated at 35°±2° C. for 42 to 48 hours, or until sufficient growth was observed.

Validation of the Non-Toxicity of the Neutralizing Median (N′)—Test “B”: A 1.0 mL aliquot of a bacterial test suspension containing approximately 1×10³ to 3×10³ CFU/mL was transferred into a sterile tube containing 9.0 mL of NF, mixed thoroughly and replaced in the water bath at 20°±1° C. Each challenge species was exposed to the neutralizing solution at 20°±1° C. for 5 minutes. Following the 5 minute exposure tine, 1.0 mL aliquots from each tube containing neutralizer/bacterial test suspension were pour-plated in duplicate using TSA+. The plates were incubated at 35°±2° C. for 42 to 48 hours, or until sufficient growth was observed.

Validation of the Inactivation of the Product (n′)—Test “A”: A 1.0 mL aliquot of TSC was transferred into a sterile tube containing 9.0 mL of the test product, mixed thoroughly and replaced in the water bath at 20°±1° C. for 5 minutes. Then a 1.0 mL aliquot was removed from the tube containing diluent/product and transferred to a sterile tube containing 8.0 mL of NF. The tube was mixed thoroughly and replaced in the water bath for 5 minutes. Then a 1.0 mL aliquot of a challenge suspension containing approximately 1×10³ to 3×10³ CFU/mL was transferred into the tube containing diluent/product/NF and mixed thoroughly. The tube was replaced in the water bath and exposed for 5 minutes. Then 1.0 mL aliquots from the tube containing diluent/product/NF/inoculum were pour-plated in duplicate using TSA+. The plates were incubated at 35°±2° C. for 42 to 48 hours, or until sufficient growth was observed.

Table 28 provides a summary of the testing methodology described above.

Data Collection: Following incubation, the colonies on the plates were counted manually, using a hand-tally counter. Counts in the range of 30 to 300 CPU were used preferentially in the data calculations.

TABLE 28
Summary of Testing Methodology

| Test A | Test B | Test C | |
|---|---|---|---|
| Neutralization | Neutralization | Neutralization | Test D |
| Validation & | Validation & | Validation & Test | Bactericidal |
| Effectiveness | Toxicity | Organism Viability | Efficacy |
| ASC-1g | NF | TSC | ASC-1g |
| TSC | Test Organism | Hold/Expose | Test Organism |
| Hold/Expose | Hold/Expose | Plate Count | Hold/Expose |
| NF | Plate Count | | Plate Count |
| Hold/Expose | | | |
| Test Organism | | | |
| Hold/Expose | | | |
| Plate Count | | | |

CALCULATIONS/REPORTING OF RESULTS: Populations of the bacterial test species were calculated in terms of the Log₁₀ Average and the CFU/ml as follows:

Log₁₀ Average=Log₁₀(Cᵢ×10^{-D})

CFU/mL=(Cᵢ×10^{-D})

where Cᵢ=Average of the 2 plates counted, and
D=Dilution factor of the plates counted.

The average CFU/mL of each Bacterial Test Suspension Control was designated as “N”. The target range for “N” was 1×10⁷ to 3×10⁷ CFU/mL of each challenge species. The average CFU/mL present in the product (post-exposure) was designated as “n” for each challenge species. In this study, the acceptance criteria is “n”≦3×10² CFU/mL of each challenge species following an exposure period to the test material of less than or equal to 5 minutes.

The average CFU/mL present in each Bacterial Test Suspension used for the Neutralizer Validation Procedure is designated “N” for each challenge species. The average CFU/mL present in each Neutralizer Toxicity Control is designated “N′” for each challenge species. The average CFU/mL present in each Neutralizer Efficacy Test is designated as “n′” for each challenge species.
The acceptance criterion for N′ is: N′≧0.5×N
The acceptance criterion for n′ is n′≧0.5×N

RESULTS: Table 29 below presents the Initial Population (CFU/mL) and the Post Exposure populations (CFU/mL) for each bacterial species, along with the exposure time.

TABLE 29
Antibacterial Activity of ASC-1g

| | Initial | | |
|---|---|---|---|
| | Population | | |
| | (CFU per mL | | Post Exposure |
| Challenge | of Test Mixture | Exposure | Population |
| Microorganism | at Time = 0) | Time | (CFU/ml) |
| Enterococcus faecium | 7.350 × 10⁶ | 30 seconds | <3.00 × 10² |
| (ATCC #6057) | Log₁₀ = 6.87 | | Log₁₀ = <2.48 |
| | | 1 minute | <3.00 × 10² |
| | | | Log₁₀ = <2.48 |
| | | 3 minutes | <3.00 × 10² |
| | | | Log₁₀ = <2.48 |
| | | 5 minutes | <3.00 × 10² |
| | | | Log₁₀ = <2.48 |
| | | 10 minutes | <3.00 × 10² |
| | | | Log₁₀ = <2.48 |
| | | 15 minutes | <3.00 × 10² |
| | | | Log₁₀ = <2.48 |
| | | 30 minutes | <3.00 × 10² |
| | | | Log₁₀ = <2.48 |
| Enterococcus faecium | 1.4350 × 10⁷ | 30 seconds | <3.00 × 10² |
| VRE, MDR | Log₁₀ = 7.16 | | Log₁₀ = <2.48 |
| (ATCC #51559) | | 1 minute | <3.00 × 10² |
| | | | Log₁₀ = <2.48 |
| | | 3 minutes | <3.00 × 10² |
| | | | Log₁₀ = <2.48 |
| | | 5 minutes | <3.00 × 10² |
| | | | Log₁₀ = <2.48 |
| | | 10 minutes | <3.00 × 10² |
| | | | Log₁₀ = <2.48 |
| | | 15 minutes | <3.00 × 10² |
| | | | Log₁₀ = <2.48 |
| | | 30 minutes | <3.00 × 10² |
| | | | Log₁₀ = <2.48 |
| Enterococcus hirae | 1.5050 × 10⁷ | 30 seconds | <3.00 × 10² |
| (ATCC #10541) | Log₁₀ = 7.18 | | Log₁₀ = <2.48 |
| | | 1 minute | <3.00 × 10² |
| | | | Log₁₀ = <2.48 |
| | | 3 minutes | <3.00 × 10² |
| | | | Log₁₀ = <2.48 |
| | | 5 minutes | <3.00 × 10² |
| | | | Log₁₀ = <2.48 |
| | | 10 minutes | <3.00 × 10² |
| | | | Log₁₀ = <2.48 |
| | | 15 minutes | <3.00 × 10² |
| | | | Log₁₀ = <2.48 |
| | | 30 minutes | <3.00 × 10² |
| | | | Log₁₀ = <2.48 |
| Escherichia coli | 1.9550 × 10⁷ | 30 seconds | <3.00 × 10² |
| (K12) | Log₁₀ = 7.29 | | Log₁₀ = <2.48 |
| (NCTC #10538) | | 1 minute | <3.00 × 10² |
| | | | Log₁₀ = <2.48 |
| | | 3 minutes | <3.00 × 10² |
| | | | Log₁₀ = <2.48 |
| | | 5 minutes | <3.00 × 10² |
| | | | Log₁₀ = <2.48 |
| | | 10 minutes | <3.00 × 10² |
| | | | Log₁₀ = <2.48 |
| | | 15 minutes | <3.00 × 10² |
| | | | Log₁₀ = <2.48 |
| | | 30 minutes | <3.00 × 10² |
| | | | Log₁₀ = <2.48 |
| Proteus mirabilis | 3.20 × 10⁷ | 30 seconds | <3.00 × 10² |
| (ATCC # 14153) | Log₁₀ = 7.51 | | Log₁₀ = <2.48 |
| | | 1 minute | <3.00 × 10² |
| | | | Log₁₀ = <2.48 |
| | | 3 minutes | <3.00 × 10² |
| | | | Log₁₀ = <2.48 |
| | | 5 minutes | <3.00 × 10² |
| | | | Log₁₀ = <2.48 |
| | | 10 minutes | <3.00 × 10² |
| | | | Log₁₀ = <2.48 |
| | | 15 minutes | <3.00 × 10² |
| | | | Log₁₀ = <2.48 |
| | | 30 minutes | <3.00 × 10² |
| | | | Log₁₀ = <2.48 |
| Pseudomonas | 1.530 × 10⁷ | 30 seconds | <3.00 × 10² |
| aeruginosa | Log₁₀ = 7.21 | | Log₁₀ = <2.48 |
| (ATCC #15442) | | 1 minute | <3.00 × 10² |
| | | | Log₁₀ = <2.48 |
| | | 3 minutes | <3.00 × 10² |
| | | | Log₁₀ = <2.48 |
| | | 5 minutes | <3.00 × 10² |
| | | | Log₁₀ = <2.48 |
| | | 10 minutes | <3.00 × 10² |
| | | | Log₁₀ = <2.48 |
| | | 15 minutes | <3.00 × 10² |
| | | | Log₁₀ = <2.48 |
| | | 30 minutes | <3.00 × 10² |
| | | | Log₁₀ = <2.48 |
| Staphylococcus aureus | 1.7050 × 10⁷ | 30 seconds | <3.00 × 10² |
| Aureus | Log₁₀ = 7.23 | | Log₁₀ = <2.48 |
| (ATCC #6538) | | 1 minute | <3.00 × 10² |
| | | | Log₁₀ = <2.48 |
| | | 3 minutes | <3.00 × 10² |
| | | | Log₁₀ = <2.48 |
| | | 5 minutes | <3.00 × 10² |
| | | | Log₁₀ = <2.48 |
| | | 10 minutes | <3.00 × 10² |
| | | | Log₁₀ = <2.48 |
| | | 15 minutes | <3.00 × 10² |
| | | | Log₁₀ = <2.48 |
| | | 30 minutes | <3.00 × 10² |
| | | | Log₁₀ = <2.48 |
| Staphylococcus aureus | 1.710 × 10⁷ | 30 seconds | <3.00 × 10² |
| MRSA | Log₁₀ = 7.23 | | Log₁₀ = <2.48 |
| (BSLI #051797MRSA2) | | 1 minute | <3.00 × 10² |
| | | | Log₁₀ = <2.48 |
| | | 3 minutes | <3.00 × 10² |
| | | | Log₁₀ = <2.48 |
| | | 5 minutes | <3.00 × 10² |
| | | | Log₁₀ = <2.48 |
| | | 10 minutes | <3.00 × 10² |
| | | | Log₁₀ = <2.48 |
| | | 15 minutes | <3.00 × 10² |
| | | | Log₁₀ = <2.48 |
| | | 30 minutes | <3.00 × 10² |
| | | | Log₁₀ = <2.48 |

These results indicate that ASC-1 rapidly kills Enterococcus faecium, Enterococcus faecium, Enterococcus hirae, Escherichia coli K12, Proteus mirabilis, Pseudomonas aeruginosa, Staphylococcus aureus aureus and Staphylococcus aureus MRSA.

### Example 12

### Evaluation of Bactericidal Activity of XX Against Staphylococcus Aureus (Subsp. Aureus Mrsa)

SUMMARY: This example demonstrates the ability of the antiseptic compositions according to the invention to kill bacteria of the genus Staphylococcus. The ability of a preparation of ASC-1 (ASC-1 g) to kill Methicillin-Resistant Staphylococcus aureus (MRSA) was assessed as follows.

NEUTRALIZATION STUDY: A neutralization study of the test product was performed versus S. aureus MRSA to ensure that the neutralizing solution used (Neutralizing Formulation) was effective in neutralizing the antimicrobial properties of the test product. This procedure follows guidelines set forth in ASTM E 1054-2 “Standard Test Methods for Evaluation of Inactivators of Antimicrobial Agents”, except that the challenge suspension will be added to the neutralizing solution prior to the addition of the product.

### Methodology:

Test Product: A preparation of ASC-1 (ASC-1 g) was prepared as described in Example 1 and used without dilution.

Bacterial Strains: The Staphylococcus aureus (MRSA) strain evaluated was American Type Culture Collection (ATCC) #700698.

Inoculum Preparation: Approximately 48 hours prior to testing, a sterile tube containing Tryptic Soy Broth (TSB) was inoculated with the MRSA. The broth culture was incubated at 35°±2° C. for approximately 24 hours, or until sufficient growth was observed. Approximately 24 hours prior to testing, the broth culture was inoculated onto the surface of Tryptic Soy Agar (TSA) contained in Petri plates and incubated at 35°±2° C. until sufficient growth was observed. These produced lawns of MRSA on the agar plates and growth from these were used to prepare the challenge suspension. The purity of the broth culture was verified by streaking for isolation on TSA and incubating it at 35°±2° C.

Challenge Suspension: Immediately prior to initiating the test procedure, a suspension of the MRSA was prepared in sterile 0.9% Sodium Chloride Irrigation. USP (SCI) by suspending the MRSA from the cultures previously prepared to achieve a challenge suspension concentration of approximately 1×10⁹CFU/ml.

Initial Population Determination: The initial population of the MRSA suspension was determined by preparing 10-fold dilutions (i.e. 10⁻¹, 10⁻², 10⁻³, 10⁻⁴, 10⁻⁵, 10⁻⁶ and 10⁻⁷) in Neutralizing Formulation (NF). The NF and product neutralizers are described in Example 5. Using TSA with product neutralizers (TSA+), pour-plates were prepared in duplicate from the inoculum dilutions by plating 0.1 mL of the final dilutions (i.e. 10⁻⁵, 10⁻⁶ and 10⁻⁷) to achieve plated dilutions of 10⁻⁶, 10⁻⁷ and 10⁻⁸. The plates were then incubated at 35°±2° C. for 48 to 72 hours, or until sufficient growth was observed.

Testing Procedure: To achieve 99% (v/v) concentration of the test product, a 0.1 mL aliquot of the MRSA suspension containing approximately 1×10⁹ CFU/ml was transferred to a sterile test tube containing 9.9 mL of the test product, ASC-1 g, and mixed thoroughly using a vortex mixer and positive displacement pipetter (10⁻² dilution). The challenge microorganism, MRSA, was then exposed to ASC-1 g for minute and 3 minutes, timed using a calibrated minute/second tinier. After each exposure time had elapsed, 1.0 mL was removed from each tube containing MRSA/challenge suspension, placed in separate sterile test tubes containing 9.0 mL of NF (10⁻³ dilution) and mixed thoroughly using a vortex mixer. Appropriate 10-fold dilutions (i.e. 10⁻⁴, 10⁻⁵ and 10⁻⁶) of the suspensions of the MRSA were prepared in NF, mixing thoroughly using a vortex mixer between dilutions. From the final dilutions of the ASC-1 g/neutralizer/challenge suspension, 1.0 mL and/or 0.1 mL aliquots were pour-plated, in duplicate, using TSA+, producing final plated dilutions of 10⁻³, 10⁻⁴, 10⁻⁵, 10⁻⁵ and 10⁻⁷. The plates were incubated at 35°±2° C. for 48-72 hours or until sufficient growth was observed.

Data Collection: Following incubation, the colonies on the plate were counted manually using a hand-tally counter. Counts in the range 30-300 CFU were used preferentially in the data calculations. If no counts in the range of 30-300 CFU were observed, then those plates with colony counts closest to that range were used for data calculations.

CALCULATIONS/REPORTING OF RESULTS: The Log₁₀ Average and the CFU/mL of the average of the duplicate plate counts for the initial population of the challenge strain and the population following each timed exposure to the test product were calculated as follows:

Log₁₀ Average=Log₁₀(Cᵢ×10^{-D})

CFU/mL=(Cᵢ×10^{-D})

where Cᵢ=Average of the 2 plates counted, and
D=Dilution factor of the plates counted.

The Log₁₀ reduction of the challenge strain attributable to the test product following each times exposure was calculated as follows:

Log₁₀Reduction=Log₁₀ IP−Log₁₀P_{EX}

where IP=Initial Population of Challenge Species (CFU/ml) and

P_{EX}=Average Population after Exposure to Each Test Product (CFU/ml)

The percent reduction of the challenge strain attributable to the test product following each timed exposure was calculated as follows:

$$Percent Reduction = \frac{IP - {P}_{EX}}{IP}$$

RESULTS: Table 30 below presents the Initial Population (CFU/ml) and the Post Exposure Population (CFU/ml) of Staphylococcus aureus aureus MRSA, along with the Log₁₀ and Percent Reduction produced by the test product at each time of exposure.

TABLE 30
Initial and post-exposure populations Staphylococcus aureus aureus
MRSA

| | | | Post | | |
|---|---|---|---|---|---|
| | Initial | | Exposure | Log₁₀ | |
| Challenge | Population | Exposure | Population | Re- | Percent |
| Microorganism | (CFU/mL) | Time | (CFU/ml) | duction | Reduction |
| Staphylococcus | 1.20 × 10⁹ | 1 minute | <1.00 × | 6.0792 | 99.9999% |
| aureus aureus | | | 10³ | | |
| MRSA | | 3 minutes | <1.00 × | 6.0792 | 99.9999% |
| (ATCC | | | 10³ | | |
| #700698) | | | | | |

MRSA = Methicillin-Resistant Staphylococcus aureus

These results indicate that ASC-1 g was able to inhibit the growth of S. aureus MRSA.

### Example 13

### Evaluation of Fungicidal Activity of XX Against Aspergillus Niger and Candida Albicans

SUMMARY: This example demonstrates the ability of an antiseptic composition according to the invention to kill fungi of mycelial and yeast species. The ability of a preparation of ASC-1 (ASC-1 g) was testing using an In vitro Time-Kill method against Aspergillus niger and Candida albicans. The In vitro Time-Kill evaluation was performed as described below using a modification of the methods described in the Draft European Standard, prEN 13624, “Chemical Disinfectants and Antiseptics—Quantitative Suspension Test for the Evaluation of Fungicidal Activity of Chemical Disinfectants for Instruments Used in the Medical Area—Test Method and Requirements (2003).”

### Methodology:

Test Product: ASC-1 g, a lot of ASC-1 prepared as described in Example 1, was used without dilution as the product test solution (i.e., full-strength).

Preparation of Asperkillis niger (ATCC #16404): 7 to 9 days prior to testing, inocula from a lyophilized vial containing A. niger were suspended in Tryptone Sodium Chloride Solution (TSC), inoculated onto the surface of Malt Extract Agar (MEA) contained in Petri plates and incubated at 30° C.±2° C. until sufficient growth was observed. This produced lawns of the challenge fungi on the surface of the agar plates and conidia from these were used to prepare the Test and Validation Suspensions.

Preparation of Candida albicans (ATCC #10231): Approximately 96 hours prior to testing, inocula from a lyophilized vial containing C. albicans were suspended in TSC, inoculated onto the surface of MEA contained in Petri plates and incubated at 30° C.±2° C. for 42 to 48 hours. 42 to 48 hours prior to testing, a suspension of C. albicans was prepared by rinsing the culture from the solid media with sterile TSC. The purity of the suspension was verified by streaking for isolation on MEA. Aliquots of the suspension were then spread-plated onto the surface of additional plates of MEA and incubated at 30° C.±2° C. for 42 to 48 hours, or until sufficient growth was observed. This produced lawns of C. albicans on the surface of the agar plates and growth from these were used to prepare the Test and Validation Suspensions.

Fungal Test (N) and Validation (Nᵥ) Suspension of Aspergillis niger: Prior to initiating the Fungicidal Assay Procedure and the Method Validation Procedure, suspensions of A. niger was prepared in a solution of sterile Water-for-Irrigation, USP, with 0.05% (v/v) Polysorbate 80 (WFI-P80). Polysorbate is used to aid in dispersal of the conidia. The conidia were detached from the surface of the solid media by gently scraping with a sterile spatula or spreader, suspended in 10 mL of WFI-P80 and transferred to a sterile flask containing glass beads. The flasks were gently shaken by hand for 1 minute and then the contents were filtered through sterile cheesecloth, as necessary, to separate the gross debris and/or mycelial fragments. The suspension was visually examined under magnification to confirm that no mycelial fragments or germinated spores were present (at least 10 fields of view were checked). If germinated spores were present, the suspension was discarded. If mycelial fragments were present, the suspensions were “washed” a minimum of 2 times by centrifuging, removing the supernatant and resuspending the pellets in TSC. The fungal spore suspension was diluted, as necessary, with additional TSC to produce a concentration of approximately 1.5×10⁷ to 5×10⁷ CFU/mL for use in the Fungicidal Assay Procedure. A suspension containing approximately 4.5×10² to 1.8×10³ CFU/mL was prepared for the Method Validation Procedure and was stored at 2° C. to 8° C. for up to 2 days prior to use, as required.

Fund Test (N) and Validation (Nᵥ) Suspension of Candida albicans: Immediately prior to initiating the Fungicidal Assay Procedure and the Method Validation Procedure, suspensions of C. albicans was prepared in a sterile TSC by suspending growth from the solid media previously prepared. The suspension was transferred to a sterile flask containing glass beads, mixed for approximately 3 minutes and transferred to sterile bottle or tube. The suspension was diluted, as necessary, with additional TSC to produce a concentration of approximately 1.5×10⁷ to 5×10⁷ CFU/mL for use in the Fungicidal Assay Procedure. A suspension containing approximately 4.5×10² to 1.8×10³ CFU/mL was prepared for the Method Validation Procedure. The adjusted suspensions were used within 2 hours of their preparation.

FUNGICIDAL ASSAY PROCEDURE: Prior to testing, the test product (ASC-1 g), the Test Suspensions, the interfering substance and all diluting fluids were placed in a water bath at 20° C. 1° C. and allowed to equilibrate for no less that 15 minutes or until the temperature of the solutions stabilized at 20° C.±1° C.

Initial Population Determination—Fungal Test Suspensions (N): A 1.0 mL aliquot of a Test Suspension containing approximately 1.5×10⁷ to 5×10⁷ CFU/mL was transferred into a sterile test tube containing 9.0 mL of TSC and mixed thoroughly (10⁻¹ dilution). Ten-fold dilutions (e.g. 10⁻², 10⁻³, 10⁻⁴, 10⁻⁵ and 10⁻⁶) of each suspension were prepared in TSC. Using Malt Extract Agar with product neutralizers (MEA+) pour or spread plates were prepared by plating duplicate 1.0 mL aliquots of the final dilutions (e.g. 10⁻⁵ and 10⁻⁶) of each Test Suspension. The product neutralizers are as described in Example 5. Note: Each 1.0 mL aliquot of suspension was divided into portions of approximately equal size and distributed into 2 plates. The plates containing C. albicans were incubated at 30° C.±2° C. for 42-48 hours, or until sufficient growth was observed. The plates containing A. niger were incubated at 30° C.±2° C. for 42-48 hours. The colonies on the plate were counted and the plates returned to incubation for an additional 20-24 hours, and counted again.

Testing Procedure—Fungicidal Test Mixture (Nₐ)—Test “D”: A 1.0 mL aliquot of a Test Suspension containing approximately 1.5×10⁷ to 5×10⁷ CFU/mL was transferred into a sterile test tube containing 1.0 mL of Bovine Albumin Solution (BAS), mixed thoroughly and replaced in the water bath at 20° C.±1° C. for 2 minutes. Then an 8.0 mL aliquot of test product was added to the tube containing BAS/Test Suspension and the tube was replaced in the water bath. Each challenge strain was exposed to the test product/BAS mixture at 20° C.±1° C. for 5 minutes, 15 minutes, 30 minutes and 60 minutes, timed using a calibrated minute/second timer. After each exposure time had elapsed, 1.0 mL was removed from each tube containing product/BAS/Test Suspension, placed in separate sterile test tubes containing 8.0 mL of Neutralizing Formulation (NF, as described in Example 5) and 1.0 mL of WFI, then mixed thoroughly using a vortex mixer. The tube was allowed to remain in the water bath for a neutralization time of 5 minutes, timed with a calibrated minute/second timer. Following the neutralization, duplicate 1.0 mL aliquots of the neutralized test mixture were pour-plated (for C. albicans) or spread-plated (for A. niger) using MEA+. The plates prepared for C. albicans were incubated at 30° C.±2° C. for 42-48 hours, or until sufficient growth was observed. The plates prepared for A. niger were incubated at 30° C.±2° C. for 42-48 hours. The colonies of the plates were counted and the plates returned to incubation for an additional 20-24 hours, then counted again.

Data Collection—V_{c} Values: Following incubation, the colonies on the plates were counted manually using a hand-tally counter. Counts in the range 14 to 330 CFU were used preferentially in the calculation of the C. albicans populations. Counts in the range of 14 to 165 were used preferentially in the calculation of the A. niger population.

METHOD VALIDATION—DILUTION-NEUTRALIZATION PROCEDURE: Fungal Validation Suspensions containing approximately 4.5×10² to 1.8×10³ CFU/mL of a challenge species were prepared by diluting the fungal spore suspensions, as necessary, with additional TSC. Prior to testing, ASC-1d, the Validation Suspensions, the interfering substance (Bovine Albumin Solution—BAS) and all diluting fluids (Neutralizing Formulation of Butterfield's Phosphate Buffer with product neutralizers as described above—NF; Tryptone Sodium Chloride Solution—TCS, and Sterile Water-for-Irrigation—WFI) were placed in a water bath at 20° C.±1° C. and allowed to equilibrate for no less than 15 minutes, or until the temperature of the solutions stabilized to 20° C.±1° C.

Initial Population Determination—Fungal Validation Suspensions (Nᵥ)—Test “C”: A 1.0 mL aliquot of a Validation Suspension containing approximately 4.5×10² to 1.8×10³ CFU/mL was transferred into a sterile tube containing 9.0 mL of TSC and mixed thoroughly. Duplicate 1.0 mL aliquots of this suspension were pour-plated (for C. albicans) or spread-plated (for A. niger) using MEA+. Note: Each 1.0 mL aliquot of suspension was be divided into portions of approximately equal size and distributed into 2 plates. The plates containing C. albicans were incubated at 30° C.±2° C. for 42-48 hours, or until sufficient growth was observed. The plates containing A. niger were incubated at 30° C.±2° C. for 42-48 hours. The colonies on the plates were counted and the plates returned to incubation for an additional 20-24 hours and counted again.

Experimental Conditions Control (A)—Test “E”: A 1.0 mL aliquot of a Validation Suspension containing approximately 4.5×10² to 1.8×10³ CFU/mL was transferred into a sterile tube containing 1.0 mL of BAS, mixed thoroughly and replaced in the water bath at 20° C.±1° C. for 2 minutes. Then an 8.0 mL aliquot of sterile WFI was added to the tube containing BAS/Validation Suspension and replaced in the water bath. Each challenge strain was then exposed to the WFI/BAS mixture at 20° C.±1° C. for 60 minutes, timed using a calibrated minute/second timer. Then duplicate 1.0 mL aliquots of this suspension were pour-plated (for C. albicans) or spread-plated (for A. niger) using MEA+. The plates containing C. albicans were incubated at 30° C.±2° C. for 42-48 hours, or until sufficient growth was observed. The plates containing A. niger were incubated at 30° C.±2° C. for 42-48 hours. The colonies on the plates were counted and the plates returned to incubation for an additional 20-24 hours and counted again.

Neutralizer Control (Validation of the Non-Toxicity of the Neutralizing Medium (B)—Test “B”: A 1.0 mL aliquot of a Validation Suspension containing approximately 4.5×10² to 1.8×10³ CFU/ml, was transferred into a sterile tube containing 8.0 mL of NF and 1.0 mL of sterile WFI, mixed thoroughly and replaced in the water bath at 20° C.±1° C. for 5 minutes. Then duplicate 1.0 mL aliquots of this suspension was divided into portions of approximately equal size and distributed into 2 plates. The plates containing C. albicans were incubated at 30° C.±2° C. for 42-48 hours, or until sufficient growth was observed. The plates containing A. niger were incubated at 30° C.±2° C. for 42-48 hours. The colonies on the plates were counted and the plates returned to incubation for an additional 20-24 hours and counted again.

Method Validation—Dilution Neutralization (C)—Test “A”: A 1.0 mL aliquot of TSC was transferred into a sterile test tube containing 1.0 mL of BAS and mixed thoroughly. An 8.0 mL of the test product was added to the TSC/BAS mixture, vortexed thoroughly and replaced in the water bath at 20° C.±1° C. for 60 minutes. Then a 1.0 mL aliquot was removed from the tube containing diluent/BAS/test product and transferred into a sterile tube containing 8.0 mL of NF. The tube was mixed thoroughly and replaced in the water bath for 5 minutes. Then a 1.0 L aliquot of a Validation Suspension containing approximately 4.5×10² to 1.8×10³ CFU/mL was transferred into the tube containing diluent/BAS/test product/NF and mixed thoroughly. The tube was then replaced in the water bath at 20° C.±1° C. for 30 minutes. Then duplicate 1.0 mL aliquots from the tube were pour-plated (for C. albicans) or spread-plated (for A. niger) using MEA+. The plates containing C. albicans were incubated at 30° C.±2° C. for 42-48 hours, or until sufficient growth was observed. The plates containing A. niger were incubated at 30° C.±2° C. for 42-48 hours. The colonies on the plates were counted and the plates returned to incubation for an additional 20-24 hours and counted again:

A summary of the testing methodology is found in Table 32 below.

Data Collection—V_{c} values: Following incubation, the colonies on the plates were counted manually using a hand-tally counter. Counts in the range of 14 to 330 CFU were used preferentially in the calculations of C. albicans. Counts in the range of 14 to 165 were used preferentially in the calculations of A. niger populations.

### Calculations/Reporting of Results:

Table 31 provide a summary of terms used in the calculation of results.

TABLE 31
Summary of Terms

| | | Number of cells per mL | Number of cells per mL in the |
|---|---|---|---|
| | Number of cells per | in the test mixture at | test mixture at the end of |
| | mL in the fungal | the beginning of | contact time (60 min for A, |
| | suspensions | contact time (Time = 0) | 5 min for B, 30 min for C) |
| Test | N - Test Suspension | Nₒ | Nₐ |
| | | (Nₒ = N/10) | |
| Controls | Nᵥ - Validation | Nᵥₒ | A, B, C |
| | Suspension | (Nᵥₒ = Nᵥ/10) | |

All plate counts were reported as V_{c}-values, which is the number of CFU counted per 1.0 mL sample. N and Nᵥ represent the fungal Test and Validation Suspensions, respectively. Nₐ represents the Fungicidal Test Mixture. A (Experimental Conditions Control), B (Neutralized Control) and C (Method Validation Control) represent the different control test mixtures. N, Nᵥ, N^{o}. N^{vo}, Nₐ and A, B, C represent the number of CFU counted per mL in the different test mixtures.

Calculation of N and Nₐ: Because 2 dilutions of the Test Suspension were evaluated, the number of CFU/ml was calculated as the weighted average count, as follows:

$$N = \frac{c}{( {n}_{1} + 0.1 {n}_{2} ) {10}^{- 5}}$$

Where c=sum of the V_{c} values taken into account
n₁=number of V_{c}-values taken into account in the lower dilutions (i.e. 10⁻⁵)
n₂=number of V_{c}-values taken into account in the higher dilutions (i.e. 10⁻⁶)
10⁻⁵=dilution factor corresponding to the lower dilution.

Nₒ is the number of CFU/mL in the test mixture at the beginning of the contact time. It is one-tenth of the weighted mean of N due to the 10-fold dilution introduced by the addition of the product and interfering substance. The target range for N is 1.5×10⁷ to 5.0×10⁷ of each challenge species. The target range for Nₒ is 1.5×10⁶ to 5.0 to 10⁶ CFU/mL of each challenge species.

Calculation of Nₐ: Nₐ is the number of survivors per mL in the test mixture at the end of the contact time and prior to neutralization. It is 10-fold higher than the V_{c}-values due to the addition of neutralizer and water, and is calculated as follows:

$${N}_{a} = \frac{c \times 10}{n}$$

where c=sum of the V_{c} values taken into account
n=number of V_{c}—values taken into account

Calculation of Nᵥ and N^{vo}: Nᵥ is the number of CFU/mL in the Validation Suspension. It is 10-fold higher than the counts in terms of V_{c}-values due to the 10⁻¹ dilution step. Nᵥₒ is the number of CFU-mL in the mixtures A, B, C at Time=0. It is one-tenth of the mean of the V_{c}-values of

$${N}_{v} = \frac{c \times 10}{n}$$

$${N}_{v o} = \frac{c}{n}$$

where c=sum of the V_{c} values taken into account
n=number of V_{c}-values taken into account

The target range for Nᵥₒ is 4.5×10¹ to 1.8×10² CFU/ml, of each challenge species.

The target range for Nᵥ is 4.5×10² to 1.8×10³ CFU/mL of each challenge species.

Calculation of A, B, C: A, B, C are the numbers of survivors in the Experimental Conditions Control, the Neutralizer Control and Method Evaluation Control at the end of the appropriate contact times.

$$A , B , C = \frac{c}{n}$$

where c=sum of the V_{c} values taken into account
n=number of V_{c}-values taken into account
   A, B and C must be equal or greater than 0.5×Nᵥₒ.

Calculation of Log₁₀ Reductions: The Log₁₀ reduction (R) of each fungal challenge species was calculated as follows:

R=Log₁₀(Nₒ/Nₐ)

TABLE 32
Summary of Testing Methodology

| | | | | Test E |
|---|---|---|---|---|
| Test A | Test B | Test C | | Neutralization |
| Neutralization | Neutralization | Neutralization | Test D | Validation & |
| Validation & | Validation & | Validation & Test | Fungicidal | Experimental |
| Effectiveness | Toxicity | Organism Viability | Efficacy | Conditions Control |
| Interfering | NF | TSC | Interfering | Interfering |
| Substance | | | Substance | Substance |
| TSC | Test Organism | Test Organism | Test Organism | Test Organism |
| ASC-1g | Hold/Expose | Plate Count | Hold/Expose | Hold/Expose |
| Hold/Expose | Plate Count | | ASC-1g | WFI |
| NF | | | Hold/Expose | Hold/Expose |
| Hold/Expose | | | NF | Plate Count |
| Test Organism | | | Hold/Expose | |
| Hold/Expose | | | Plate Count | |
| Plate Count | | | | |

RESULTS: Table 33 below presents the Initial Population (CFU/ml) and the Post Exposure Population (CFU/ml) of Aspergillus niger and Candida albicans, along with the Log₁₀ and Percent Reduction produced by the test product at each time of exposure.

TABLE 33
Fungicidal Activity of ASC-1g

| | Initial Population | | | |
|---|---|---|---|---|
| | (CFU per mL of | | Post Exposure | |
| Challenge | Test Mixture | Exposure | Population | Log₁₀ |
| Microorganism | at Time = 0) | Time | (CFU/ml) | Reduction |
| Aspergillis niger | 1.1 × 10⁶ | 5 minutes | 5.1 × 10² | 3.33 |
| (ATCC #16404) | Log₁₀ = 6.04 | | Log₁₀ = 2.71 | |
| Test Suspension | | 15 minutes | <1.4 × 10² | >3.89 |
| Population = | | | Log₁₀ = <2.15 | |
| 1.1 × 10⁷ CFU/mL | | 30 minutes | <1.4 × 10² | >3.89 |
| | | | Log₁₀ = <2.15 | |
| | | 60 minutes | <1.4 × 10² | >3.89 |
| | | | Log₁₀ = <2.15 | |
| Candida albicans | 2.1 × 10⁶ | 5 minutes | <1.4 × 10² | >4.17 |
| (ATCC #10231) | Log₁₀ = 6.32 | | Log₁₀ = <2.15 | |
| | | 15 minutes | <1.4 × 10² | >4.17 |
| | | | Log₁₀ = <2.15 | |
| | | 30 minutes | <1.4 × 10² | >4.17 |
| | | | Log₁₀ = <2.15 | |
| | | 60 minutes | <1.4 × 10² | >4.17 |
| | | | Log₁₀ = <2.15 | |

The results of this study indicate that ASC-1 g is effective against A. niger within 15 minutes of exposure, while ASC-1 g is effective against C. albicans within 5 minutes of exposure.

### Example 14

### Evaluation of Neutralization Formula for ASC-1 in Antibacterial Studies

SUMMARY: This study evaluated the ability of a neutralization formula (Butterfield's Phosphate Buffer Solution with product neutralizers as described in Example 5) to inactivate the antibacterial activity of an antiseptic composition (ASC-1 g). This neutralization formula was used in the studies described in the Examples where indicated. Two bacterial species were used for this evaluation, Escherichia coli (K12) (NCTC #10538) and Staphylococcus aureus (ATCC #6538). Testing followed the guidelines set forth in ASTM E 1054-02, “Standard Test Methods for Evaluations of Inactivators of Antimicrobial Agents.” This specifies that the neutralization is considered adequate and the neutralizing medium considered non-toxic if the Log₁₀ numbers of survivors of each bacterial species form each phase of the validation and non statistically different (one-way ANOVA, α=0.05) or differ by no more than 0.2 Log₁₀.

### Methodology:

Test Product: A preparation of ASC-1 (ASC-1 g), made as described in Example 1.

Inoculum Preparation: Approximately 48 hours prior to testing, separate sterile tubes containing Tryptic Soy Broth (TSB) were from lyophilized vials or cryogenic stock cultures containing the challenge species, E. coli and S. aureus. The broth cultures were incubated at 35° 2° C. for approximately 24 hours, or until sufficient growth was observed. Approximately 24 hours prior to testing, the broth cultures were used to inoculate the surface of Tryptic Soy Agar (TSA) contained in Petri plates and then incubated at 35°±2° C. until sufficient growth was observed. These produced lawns of the bacteria on the surface of the agar plates and bacteria from these were used to prepare the challenge suspensions. The purity of each broth culture was verified by streaking for isolation on TSA and incubating it at 35°±2° C. for 24-72 hours.

Challenge Suspensions: Immediately prior to initiating the test procedure, a suspension of each bacterial species was prepared in sterile 0.9% Sodium Chloride Irrigation USP (SCI) by suspending the growth from the TSA cultures to achieve concentrations of approximately 1×10⁹ CFU/mL as determined visually on the basis of comparison to a #6 McFarland Standard. Final challenge suspensions containing approximately 1×10³ CFU/mL of each bacterial species was created by preparing 10-fold serial dilutions of the initial suspensions in additional SCI.

Test A-Neutralizer Effectiveness: Three replicates of this procedure were performed versus each challenge species. A 0.1 mL aliquot of a challenge suspension containing approximately 1×10³ CFU/mL was added to a sterile test tube containing 8.9 mL of Neutralizing Formula (NF) as described in Example 5 and mixed thoroughly. Within 5 seconds, a 1.0 mL aliquot of the test product (ASC-1 g), was added to the tube containing inoculum/NF and mixed thoroughly. Immediately (i.e. with 1 minute) following the addition of the test product, 1.0 mL aliquots of the neutralizing mixture were pour-plated, using Tryptic Soy Agar with product neutralizers as described in Example 5 (TSA+). The test product/NF/inoculum suspension was allowed to stand for 15 minutes, timed with a calibrated minute/second timer. After the 15 minute “hold” time, 1.0 mL aliquots of the neutralization mixture were pour-plated in duplicate using TSA+. The plates were then incubated at 35°±2° C. for 24-72 hours, or until sufficient growth was observed.

Test B—Neutralizer Toxicity: Three replicates of this procedure were performed versus each challenge species. A 0.1 mL aliquot of a challenge suspension containing approximately 1×10³ CFU/mL was added to a sterile test tube containing 8.9 mL of Neutralizing Formula (NF) and mixed thoroughly. Within 5 seconds, a 1.0 mL aliquot of Phosphate Buffered Saline (PBS) was added to the tube containing inoculum/NF and mixed thoroughly. Immediately (i.e. with 1 minute) following the addition of the PBS, 1.0 mL aliquots of the neutralization/inoculum mixture were pour-plated, using TSA+. The PBS/NF/inoculum suspension was allowed to stand for 15 minutes, timed with a calibrated minute/second timer. After the 15 minute “hold” time, 1.0 mL aliquots of the neutralization inoculum mixture were pour-plated, in duplicate, using TSA+. The plates were then incubated at 35°±2° C. for 24-72 hours, or until sufficient growth was observed.

Test C—Bacterial Viability: Three replicates of this procedure were performed versus each challenge species. A 0.1 mL aliquot of a challenge suspension containing approximately 1×10³ CFU/mL was transferred to a sterile test tube containing 9.9 mL of PBS and mixed thoroughly. Immediately (i.e. with 1 minute) following inoculation, 1.0 mL aliquots of the PBS/inoculum mixture were pour-plated, in duplicate, using TSA+. The PBS/inoculum suspension was allowed to stand for 15 minutes, timed with a calibrated minute/second timer. After the 15 minute “hold” time, 1.0 mL aliquots of the mixture were pour-plated in duplicate using TSA+. The plates were then incubated at 35°±2° C. for 24-72 hours, or until sufficient growth was observed.

A summary of the steps in each of Test A, Test B and Test C is shown in Table 34 below.

TABLE 34
Summary of Terms

| Test A | Test B | Test C |
|---|---|---|
| Neutralizer Effectiveness | Neutralizer Toxicity | Test Organism Viability |
| T³6 Technology | PBS | N/A |
| NF | NF | PBS |
| Test Organism | Test Organism | Test Organism |
| Plate Count | Plate Count | Plate Count |
| Hold | Hold | Hold |
| Plate Count | Plate Count | Plate Count |

CALCULATIONS/INTERPRETATION OF DATA: Following incubation, the colonies on the plates were counted manually using a hand-tally counter. The number of surviving bacteria was calculated for each replicate from each test using the following formula:

$$Number of Survivors = \frac{plate count 1 + plate count 2}{2}$$

The values obtained were converted to Log₁₀ values.

Statistical Analysis: The Log₁₀ of the number of survivors of each bacterial species from Tests A and B were statistically compares to those from Test C (Bacterial Viability Population) using a One-Way Analysis of Variance (ANOVA). If the Test A and Test B 95% Confidence Intervals overlap with the Test C 95% Confidence Interval, then all of the populations were considered equivalent; the Neutralizing Formulation will be considered to be effective in neutralizing the antibacterial activity of the test product and non-toxic to the challenge bacterial species.

Alternative Method for Assessing Test A and Test B Outcomes: Since the variability of the test data can affect interpretation of the results from the statistical analysis, an alternative method of assessing the neutralization outcomes may be necessary for sample populations which do not differ significantly. As referenced by ASTM E 1054-2, the Neutralizing Formulation may be considered to be effective in neutralizing ASC-1 g and non-toxic to the challenge species if the Test A and Test 13 microbial recovery populations (i.e. average of the 3 replicates) are no more than 0.2 Log₁₀ lower than the Test C population (i.e. average of the 3 replicates).

RESULTS: Table 35 presents the Log₁₀ microbial populations of Escherichia coli resulting from each phase of the Neutralization Validation. Table 36 presents the Logₒ microbial populations of S. aureus resulting from each phase of the Neutralization Validation

### Neutralization Validation Results

TABLE 35
Challenge Bacterial Species Escherichia coli
(K12) (NCTC #10538)

| Contact | | Log₁₀ Microbial Population | | | Results |
|---|---|---|---|---|---|
| Time | Replicate | Test A | Test B | Test C | (Pass/Fail) |
| Time = 0 | 1 | 1.7889 | 1.7243 | 1.7404 | Pass¹ |
| | 2 | 1.6767 | 1.7709 | 1.7924 | Pass¹ |
| | 3 | 1.7202 | 1.6902 | 1.7202 | Pass¹ |
| Time = 15 | 1 | 1.6721 | 1.6580 | 1.6902 | Pass¹ |
| minutes | 2 | 1.7709 | 1.6767 | 1.7033 | Pass¹ |
| | 3 | 1.7599 | 1.7597 | 1.6128 | Pass¹ |

TABLE 36
Challenge Bacterial Species Staphylococcus aureus (ATCC #6538)

| Contact | | Log₁₀ Microbial Population | | | Results |
|---|---|---|---|---|---|
| Time | Replicate | Test A | Test B | Test C | (Pass/Fail) |
| Time = 0 | 1 | 1.9638 | 1.9685 | 1.9708 | Pass¹ |
| | 2 | 1.9165 | 1.9165 | 1.9294 | Pass¹ |
| | 3 | 1.9566 | 1.8865 | 1.9590 | Pass¹ |
| Time = 15 | 1 | 2.0212 | 1.9085 | 1.8779 | Pass¹ |
| minutes | 2 | 1.9191 | 1.8722 | 1.9294 | Pass¹ |
| | 3 | 1.9542 | 1.9269 | 1.8779 | Pass¹ |

NOTE:
Pass¹ = Results of Test A and Test B are no more than 0.2 Log₁₀ lower than Test C and/or the 95% Confidence Intervals (α = 0.05) of Test A and Test B overlap that of Test C, where Test A = Neutralizer Effectiveness, Test B = Neutralizer Toxicity and Test C = Bacterial Viability Controls versus ASC-1g.

The preceding results indicate that the Neutralization Formula was effective in inactivating ASC-1 g.

### Example 15

### Evaluation of Acute Oral Toxicity of ASC-1 in Rats

This example demonstrates that the antiseptic compositions of the invention are not toxic when orally administered. The oral toxicity of an antiseptic composition according to the invention was determined by short-term exposure of rats to ASC-1 by the oral route.

SUMMARY: An acute oral toxicity test was conducted with rats to determine the potential for ASC-1 to produce toxicity from a single dose via the oral route. Based on the results of this study, the single dose acute oral LD₅₀ of the test substance is greater than 5,000 mg/kg of bodyweight.

In this study, 5,000 mg of the test substance per kilogram of bodyweight was administered to 10 healthy rats by oral gavage. The animals were observed for mortality, signs of gross toxicity, and behavioral changes at least once daily for 14 days. Bodyweights were recorded prior to administration and again on Days 7 and 14 (termination). Necropsies were performed on all animals at terminal sacrifice.

All animals survived and gained weight during the study. Following administration, most animals exhibited piloerection, hunched posture and/or were hypoactive. Apart from one female that exhibited reduced fecal volume between Days 0 and 5, all affected animals recovered from the above symptoms within 21 hours and appeared active and healthy for the remainder of the 14-day observation period. Gross necropsy findings at terminal sacrifice were unremarkable.

### Materials:

Test Substance: The test substance was ASC-1, prepared as described in Example 1 and identified as ASC-1c. The test substance was a clear liquid, was stored at room temperature, and was not diluted prior to use. The test substance had a pH of 7.2 and was soluble in water and ethanol. The test substance was stable for the duration of testing.

### Animals:

### Number of Animals: 10

Sex: 5 male and 5 female
Species/Strain: Rat/Sprague-Dawley derived, albino
Age/Bodyweight: Young adult/males 227-253 grams and females 173-197 grams.
Source: Received from Ace Animals, Inc., Boyertown, Pa.

### Methods:

Selection of Animals: Prior to dosing, a group of animals was fasted for approximately 18.5 hours by removing feed from their cages. During the fasting period, the rats were examined for health and weighed (initial). Ten (five male and five female) healthy rats were selected for test.

Dose Calculations: Individual doses were calculated based on the initial bodyweights, taking into account the specific gravity of the test substance.

Dosing: Each animal received 5,000 mg/kg of the test substance, administered using a stainless steel ball-tipped gavage needle attached to an appropriate syringe. After administration, each animal was returned to its designated cage. Feed was replaced approximately three hours after dosing. The day of administration was considered Day zero of the study.

Bodyweights: Individual bodyweights of the animals were recorded prior to test substance administration (initial) and again on Days 7 and 14 (termination) (See Table 37).

Cage-Side Observations: The animals were observed for mortality, signs of gross toxicity, and behavioral changes at 1, 3 and 21 hours post-dosing, and at least once daily thereafter for 14 days. Observations included gross evaluation of skin and fur, eyes and mucous membranes, respiratory, circulatory, autonomic and central nervous systems, somatomotor activity and behavior pattern. Particular attention was directed to observation of tremors, convulsions, salivation, diarrhea and coma (See Table 38).

Necropsy: All rats were euthanized via CO₂ inhalation on Day 14. Gross necropsies were performed on all animals. Tissues and organs of the thoracic and abdominal cavities were examined (Sec Table 39).

RESULTS: All animals survived and gained weight during the study. Following administration, most animals exhibited piloerection, hunched posture and/or were hypoactive. Apart from one female that exhibited reduced fecal volume between Days 0 and 5, all affected animals recovered from the above symptoms within 21 hours and appeared active and healthy for the remainder of the 14-day observation period. No gross abnormalities were noted for the animals when necropsied at the conclusion of the study.

CONCLUSION: The single dose acute oral LD₅₀ of ASC-1c is greater than 5,000 mg/kg of bodyweight.

TABLE 37
Individual Bodyweights And Doses

| Animal | | Bodyweight (g) | | | Dose |
|---|---|---|---|---|---|
| No. | Sex | Initial | Day 7 | Day 14 | mL |
| 9837 | M | 253 | 287 | 373 | 1.4 |
| 9838 | M | 227 | 266 | 329 | 1.3 |
| 9839 | M | 236 | 271 | 348 | 1.3 |
| 9840 | M | 232 | 269 | 349 | 1.3 |
| 9841 | M | 227 | 266 | 305 | 1.3 |
| 9842 | F | 173 | 199 | 225 | 0.98 |
| 9843 | F | 175 | 204 | 224 | 1.0 |
| 9844 | F | 197 | 212 | 245 | 1.1 |
| 9845 | F | 187 | 200 | 235 | 1.1 |
| 9846 | F | 194 | 218 | 226 | 1.1 |

TABLE 38
Individual Cage-Side Observations

| Animal | | |
|---|---|---|
| Number | Findings | Day of Occurrence |
| MALES: | | |
| 9837 | Hypoactive | 0 (1-3 hr) |
| | Hunched posture | 0 (3 hr) |
| | Active and healthy | 0 (21 hr) - 14 |
| 9838 | Hypoactive | 0 (1-3 hr) |
| | Piloerection | 0 (3 hr) |
| | Active and healthy | 0 (21 hr) - 14 |
| 9839, 9840 | Active and healthy | 0 (1 hr) - 14 |
| 9841 | Hypoactive | 0 (1-3 hr) |
| | Active and healthy | 0 (21 hr) - 14 |
| FEMALES: | | |
| 9842, 9844 | Hypoactive | 0 (1-3 hr) |
| | Active and healthy | 0 (21 hr) - 14 |
| 9843 | Hypoactive | 0 (1-3 hr) |
| | Piloerection | 0 (3 hr) |
| | Active and healthy | 0 (21 hr)-14 |
| 9845 | Active and healthy | 0 (1 hr), 0(21 hr)-14 |
| | Hypoactive | 0 (3 hr) |
| 9846 | Active and healthy | 0 (1 hr)-2, 6-14 |
| | Reduced fecal volume | 3-5 |

TABLE 39
Individual Necropsy Observations

| Animal | | |
|---|---|---|
| Number | Tissue | Findings |
| MALES: | | |
| 9837-9841 | All tissues/organs | No gross abnormalities |
| FEMALES: | | |
| 9842-9846 | All tissues/organs | No gross abnormalities |

### Example 16

### Acute Inhalation Toxicity of ASC-1 in Rats

This example demonstrates that the antiseptic compositions according to the invention are not toxic at the dose tested. The toxicity of an antiseptic composition according to the invention, ASC-1, was determined by exposing rats to ASC-1 via the inhalation route.

SUMMARY: An acute inhalation toxicity test was conducted with rats to determine the potential for ASDC-1 to produce toxicity from a single exposure via the inhalation route. Based on the results of this study, the single exposure acute inhalation LC₅₀ of the test substance is greater than 2.02 mg/L.

After establishing the desired generation procedures during pre-test trials, ten healthy rats were exposed to the test atmosphere for 4 hours. Chamber concentration and particle size distributions of the test substance were determined periodically during the exposure period. The animals were observed for mortality, signs of gross toxicity, and behavioral changes at least once daily for 14 days. Bodyweights were recorded prior to exposure and again on Days 7 and 14 (termination). Necropsies were performed on all animals at terminal sacrifice.

All animals survived exposure to the test atmosphere and gained bodyweight over the 14-day observation period. The gravimetric chamber concentration was 2.02 mg/L. Based on graphic analysis of the particle size distribution as measured with an Andersen Cascade Impactor, the mass median aerodynamic diameter was estimated to be 1.6 microns.

In-chamber animal observations included ocular and nasal discharge, dyspnea, irregular respiration, shallow respiration, hunched posture and hypoactivity. With the exception of ocular and nasal discharge and shallow respiration, similar clinical signs persisted in all animals upon removal from the exposure chamber. Some animals also developed rales, reduced fecal volume and/or a prone posture, but all rats recovered from these symptoms by Day 11 and appeared active and healthy for the remainder of the study. Gross necropsy findings at terminal sacrifice were unremarkable.

### Materials:

Test Substance: The test substance was ASC-1, prepared as described in Example 1 and identified as ASC-1c. The test substance was a clear liquid, was stored at room temperature, and was not diluted prior to use. The test substance had a pH of 7.2 and was soluble in water and ethanol. The test substance was stable for the duration of testing.

### Animals:

### Number of Animals: 10

Sex: 5 male and 5 female
Species/Strain: Rat/Sprague-Dawley derived, albino
Age/Bodyweight: Young adult/males 235-275 grams and females 210-222 grams.
Source: Received from Ace Animals, Inc., Boyertown, Pa.

### Methods:

Pre-Test Trials: Prior to initiation of the full inhalation study, pre-test trials were conducted to establish generation procedures for achieving as closely as possible the desired chamber concentration (2.0 mg/L) and desired particle size distribution (mass median aerodynamic diameter less than or equal to 4 μm). In these trials, the following adjustments were made in an attempt to achieve these objectives:
Air Pressure: constant; Compressed Airflow: constant; Room Airflow: constant;
Total Airflow: constant; Pump Setting: varied; Tube Size: constant; Atomization
System: constant; Fluid Cap: constant; and Air Cap: constant.

The exposure procedures and atomization equipment used were based on the results of pre-test trial number 3 which provided a gravimetric concentration of 2.08 mg/L and a mass median aerodynamic diameter of 1.7 μm. Details of all trials, the equipment used and the adjustments made are described in Tables 40-42.

Inhalation Procedures: The exposure chamber, air supply and equipment used to measure particle size distribution, airflow and chamber concentration were the same as used during the pre-test trials and are described below.
1. Exposure Chamber: Rectangular whole body Plexiglas® chamber with a volume of 150 liters with the prechamber operated under slight negative pressure.
2. Air Supply: Approximately 20.0 liters per minute (Lpm) of filtered air was supplied by an air compressor (JUN-AIR) to the spray atomization nozzle. Compressed airflow was measured with an Omega Mass Flowmeter Model #FMA 5613. Approximately 20.3 Lpm of filtered conditioned room air was supplied as diluent air. Room airflow was measured with an Omega Mass Flowmeter, Model #FMA 5613. Chamber airflow was monitored throughout the exposure period and recorded periodically. Total airflow ranged from 40.1 to 40.5 with a mean of 40.3 Lpm.
3. Ambient Conditions: The chamber temperature and relative humidity ranges during exposure were 21-22° C. and 40-100% RH, respectively. The room temperature and relative humidity ranges during exposure were 21-22° C. and 33-39% RH, respectively. In-chamber measurements were made with a Taylor Humidity-Temperature Indicator 5502 and room conditions were measured with a Dickson Temperature-Humidity Monitor Model TH550. Temperature and humidity values were recorded every 15 minutes for the first hour of exposure and every 30 minutes thereafter.
4. Atmosphere Generation: The test atmosphere was generated using a ¼ inch JCO atomizer, FC4 fluid cap and AC1502 air cap (Spraying Systems Inc.). Compressed air was supplied at 25 psi. The test substance was metered to the atomization nozzle through Size 14 Master Flex Tygon tubing, using a Master Flex Pump Model #7520-35.
5. Chamber Concentration Measurements: Gravimetric samples were withdrawn at six intervals from the breathing zone of the animals. Samples were collected using 25 mm glass fiber filters (GF/B Whatman) in a filter holder attached by ¼ inch tygon tubing to an Emerson Electric vacuum pump Model #S55NXMLD-6711. Filter papers were weighed before and after collection to determine the mass collected. This value was divided by the total volume of air sampled to determine the chamber concentration (Table 43). The collections were carried out for 3 minutes at airflows of 4 Lpm. Sample airflows were measured using an Omega Flowmeter Model #FMA 5610.
6. Particle Size Distribution: An eight-stage Andersen cascade impactor was used to assess the particle size distribution of the test atmosphere. Samples were withdrawn from the breathing zone of the animals at two intervals. The filter paper collection stages were weighed before and after sampling to determine the mass collected upon each stage (Table 44). The aerodynamic mass median diameter and geometric standard deviation were determined graphically using two-cycle logarithmic probit axes (Table 45).
7. Exposure Period: The animals were exposed to the test atmosphere for 4 hours and 17 minutes. The exposure period was extended beyond 4 hours to allow the chamber to reach equilibrium (T99). The times for 90 and 99% equilibration of the chamber atmosphere were 8.6 and 17.1 minutes, respectively. At the end of the exposure period, the generation was terminated and the chamber was operated for a further 18 minutes with clean air. At the end of this period the animals were removed from the chamber. Prior to being returned to their cages, excess test substance was removed from the fur of each animal.

Selection of Animals: On the day of and prior to exposure, the rats were examined for health and weighed. Ten (five male and five female) healthy rats were selected for test.

Bodyweights: Individual bodyweights of the animals were recorded prior to test substance exposure (initial) and again on Days 7 and 14 (termination) (Table 46).

Cage-Side Observations: The animals were observed for mortality, signs of gross toxicity, and behavioral changes prior to exposure, at least every 30 minutes during exposure, upon removal from the exposure chamber and at least once daily thereafter for 14 days. Observations included gross evaluation of skin and fur, eyes and mucous membranes, respiratory, circulatory, autonomic and central nervous systems, somatomotor activity and behavior pattern. Particular attention was directed to observation of tremors, convulsions, salivation, diarrhea and coma (Table 47).

Necropsy: All rats were euthanized via CO2 inhalation on Day 14. Gross necropsies were performed on all animals. Tissues and organs of the thoracic and abdominal cavities were examined (Table 48).

RESULTS: All animals survived exposure to the test atmosphere and gained bodyweight over the 14-day observation period. The gravimetric and nominal chamber concentrations were 2.02 and 78.51 mg/L, respectively. The mass median aerodynamic diameter was estimated to be 1.6 microns based on the particle size distribution as measured with an Andersen Cascade Impactor. In-chamber animal observations included ocular and nasal discharge, dyspnea, irregular respiration, shallow respiration, hunched posture and hypoactivity. With the exception of ocular and nasal discharge and shallow respiration, similar clinical signs persisted in all animals upon removal from the exposure chamber. Some animals also developed rales, reduced fecal volume and/or a prone posture, but all rats recovered from these symptoms by Day 11 and appeared active and healthy for the remainder of the study. No gross abnormalities were noted for the animals when necropsied at study termination.

CONCLUSION: The single exposure acute inhalation LC₅₀ of ASC-1c is greater than 2.02 mg/L.

TABLE 40
Preparation and Generation System for Pre-Test Trials

| Components | Manufacturer & Model |
|---|---|
| Pump | Master Flex, Model #7520-35 |
| Tubing for Pump | Tygon, Size 14 |
| Air Supply | JUN-AIR Compressor |
| Atomization | Spraying System Inc. ¼″ JCO |
| | Atomizer |
| Fluid Cap | FC4 |
| Air Cap | AC1502 |
| Chamber | 150 L Plexiglass with Prechamber |
| Diluent Airflow | Omega Mass Flowmeter, Model |
| Measurements | #FMA 5613 |

TABLE 41
Pre-Test Exposure Trials

| | | | Room | | | | |
|---|---|---|---|---|---|---|---|
| | Compressed | Compressed | Air | Total Air | | Chamber | Particle |
| Trial | Air Pressure | Air Volume | Volume | Volume | Pump | Conc. | Size |
| No. | (psi) | (Lpm) | (Lpm) | (Lpm) | Setting | (mg/L) | Sampled |
| 1 | 25 | 20.1 | 20.3 | 40.4 | 12.0 | 1.58 | No |
| 2 | 25 | 20.0 | 20.3 | 40.2 | 14.0 | 2.58 | No |
| 3 | 25 | 20.1 | 20.3 | 40.4 | 13.0 | 2.08 | Yes |

TABLE 42
Summary of Pre-Test Exposure Trials

| Trials | Chamber | Mass Medium |
|---|---|---|
| No. | Concentration (mg/L) | Aerodynamic Diameter (μm) |
| 3 | 2.08 | 1.7 |

TABLE 43
Gravimetric Chamber Concentrations

| | | Mass | Airflow | Collection | Chamber |
|---|---|---|---|---|---|
| Sample | Time of | Collected | Sampled | Time | Concentration |
| No. | Sample | (mg) | (Lpm) | (min) | (mg/L) |
| 1 | 0.5 hour | 21.7 | 4 | | 1.81 |
| 2 | 1.0 hour | 24.5 | 4 | | 2.04 |
| 3 | 2.0 hour | 24.7 | 4 | | 2.06 |
| 4 | 2.5 hour | 25.2 | 4 | | 2.10 |
| 5 | 3.5 hour | 24.6 | 4 | | 2.05 |
| 6 | 4.0 hour | 24.8 | 4 | | 2.07 |

| | |
|---|---|
| Average ± Standard Deviation | 2.02 ± 0.11 |

TABLE 44
Particle Size Distribution

| | | | |
|---|---|---|---|
| | Effective Cutoff | % of Total | |
| | Diameter | Particles Captured | Cumulative |
| Stage | (μm) | (by weight) | (%)¹ |

Sample 1

| | | | |
|---|---|---|---|
| 0 | 9.0 | 1.0 | 99.0 |
| 1 | 5.8 | 3.1 | 95.8 |
| 2 | 4.7 | 0.7 | 95.1 |
| 3 | 3.3 | 5.6 | 89.5 |
| 4 | 2.1 | 33.4 | 56.1 |
| 5 | 1.1 | 31.4 | 24.7 |
| 6 | 0.7 | 13.6 | 11.1 |
| 7 | 0.4 | 7.0 | 4.2 |
| F | 0.0 | 4.2 | 0.0 |

Sample 2

| | | | |
|---|---|---|---|
| 0 | 9.0 | 1.1 | 98.9 |
| 1 | 5.8 | 2.2 | 96.8 |
| 2 | 4.7 | 2.2 | 94.6 |
| 3 | 3.3 | 5.1 | 89.5 |
| 4 | 2.1 | 29.2 | 60.3 |
| 5 | 1.1 | 32.1 | 28.2 |
| 6 | 0.7 | 18.1 | 10.1 |
| 7 | 0.4 | 7.9 | 2.2 |
| F | 0.0 | 2.2 | 0.0 |

¹= Percent of particles smaller than corresponding effective cutoff diameter.

TABLE 45
Summary Of Particle Size Distribution

| | | Collection | Mass Median | Geometric |
|---|---|---|---|---|
| Sample | Time of | Time | Aerodynamic | Standard |
| No. | Sample | (minutes) | Diameter (μm) | Deviation |
| 1 | 1.5 hours | 10 | 1.6 | 2.04 |
| 2 | 3.0 hours | 10 | 1.6 | 1.95 |

TABLE 46
Individual Bodyweights

| | |
|---|---|
| Animal | Bodyweight |

| No. | Sex | Initial | Day 7 | Day 14 |
|---|---|---|---|---|
| 61 | M | 275 | 318 | [symbol] 75 |
| 62 | M | 235 | 278 | [symbol] 15 |
| 63 | M | 254 | 257 | [symbol] 30 |
| 64 | M | 252 | 300 | [symbol] 41 |
| 65 | M | 267 | 329 | [symbol] 03 |
| 66 | F | 213 | 219 | [symbol] 65 |
| 67 | F | 214 | 230 | [symbol] 72 |
| 68 | F | 222 | 227 | [symbol] 47 |
| 69 | F | 210 | 231 | [symbol] 48 |
| 70 | F | 213 | 219 | [symbol] 45 |

[symbol]

indicates data missing or illegible when filed

TABLE 47
Individual Cage side observations

| Animal | | Day of |
|---|---|---|
| Number | Findings | Occurrence |
| MALES: | | |
| 61 | Dyspnea, rales (moist) | CR¹-0 (1 hr) |
| | Irregular respiration, hunched posture | CR-O (16 hr) |
| | Hypoactive | CR-1 |
| | Active and healthy | 2-14 |
| 62 | Dyspnea | CR-0 (1 hr) |
| | Hunched posture, hypoactive | CR-1 |
| | Irregular respiration | CR-3 |
| | Active and healthy | 4-14 |
| 63 | Dyspnea | CR-0 (1 hr) |
| | Hunched posture | CR-0 (16 hr) |
| | Hypoactive | CR-1 |
| | Irregular respiration | CR-3, 7-10 |
| | Active and healthy | 4-6, 11-14 |
| | Rales (moist) | 7-10 |
| 64 | Dyspnea, hunched posture | CR-0 (1 hr) |
| | Hypoactive | CR-2 |
| | Irregular respiration | CR-3 |
| | Active and healthy | 4-14 |
| 65 | Dyspnea, hunched posture | CR-0 (1 hr) |
| | Irregular respiration | CR-0 (16 hr) |
| | Hypoactive | CR-1 |
| | Active and healthy | 2-14 |
| FEMALES: | | |
| 66 | Dyspnea, rales (moist) | CR¹-0 (16 hr) |
| | Hunched posture, hypoactive | CR-1 |
| | Irregular respiration | CR-3 |
| | Reduced fecal volume | 0 (16 hr) -2 |
| | Active and healthy | 4-14 |
| 67 | Prone | CR |
| | Dyspnea | CR-0 (1 hr) |
| | Hypoactive | CR-2 |
| | Irregular respiration | CR-3 |
| | Hunched posture | 0 (1 hr) |
| | Active and healthy | 4-14 |
| 68 | Dyspnea, hunched posture | CR-0 (1 hr) |
| | Irregular respiration, hypoactive | CR-0 (16 hr) |
| | Active and healthy | 1-14 |
| 69 | Irregular respiration, dyspnea, | CR-0 (1 hr) |
| | hunched posture. | |
| | Hypoactive | CR-0 (16 hr) |
| | Active and healthy | 1-14 |
| 70 | Dyspnea, hunched posture | CR-0 (1 hr) |
| | Irregular respiration, hypoactive | CR-0 (16 hr) |
| | Active and healthy | 1, 4-14 |
| | Reduced fecal volume | 2-3 |

TABLE 48
Individual Necropsy Observations

| Animal | | |
|---|---|---|
| Number | Tissue | Findings |
| MALES: | | |
| 61-65 | All tissues/organs | No gross abnormalities |
| FEMALES: | | |
| 66-70 | All tissues/organs | No gross abnormalities |

### Example 17

### Acute Dermal Toxicity of ASC-1 in Rats

This example indicates that toxicity of the antiseptic compositions of the invention are not toxic when applied topically. The dermal toxicity of an antiseptic composition according to the invention was tested by short-term exposure of rats to ASC-1 by the dermal route.

SUMMARY: An acute dermal toxicity test was conducted with rats to determine the potential for ASC-1 to produce toxicity from a single topical application. Based on the results of this study, the single dose acute dermal LD₅₀ of the test substance is greater than 2,000 mg/kg of bodyweight. In this study 2000 mg/kg of bodyweight of the test substance was applied to the skin of ten healthy rats for 24 hours. The animals were observed for mortality, signs of gross toxicity, and behavioral changes at least once daily for 14 days. Bodyweights were recorded prior to application and on Days 7 and 14 (termination). Necropsies were performed on all animals at terminal sacrifice. All animals survived, gained weight and appeared active and healthy. There were no signs of gross toxicity, adverse pharmacologic effects or abnormal behavior. Gross necropsy findings at terminal sacrifice were unremarkable.

### Materials:

Test Substance: The test substance was ASC-1, prepared as described in Example 1 and identified as ASC-1c. The test substance was a clear liquid, was stored at room temperature, and was not diluted prior to use. The test substance had a pH of 7.2 and was soluble in water and ethanol. The test substance was stable for the duration of testing.

### Animals:

### Number of Animals: 10

Sex: 5 male and 5 female
Species/Strain: Rat/Sprague-Dawley derived, albino
Age/Bodyweight: Young adult/males 227-253 grams and females 173-197 grams.
Source: Received from Ace Animals, Inc., Boyertown, Pa.

### Methods:

Selection of Animals: On the day prior to application, a group of animals was prepared by clipping (Oster model #A5-small) the dorsal area and the trunk. After clipping and prior to application, the animals were examined for health, weighed (initial) and the skin checked for any abnormalities. Ten (5 male and 5 female) healthy rats were selected for the test.

Dose Calculations: Individual doses were calculated based on the initial bodyweights, taking into account the specific gravity of the test substance.

Application of Test Substance: 2000 mg/kg of bodyweight of the test substance was applied evenly over a dose area of approximately 2 inches×3 inches (approximately 10% of the body surface) and covered with a 2 inch×3 inch, 4-ply gauze pad. The gauze pad and entire trunk of each animal were then wrapped with 3 inch Durapore tape to avoid dislocation of the pad and to minimize loss of the test substance. The rats were then returned to their designated cages. The day of application was considered Day zero of the study. After 24 hours of exposure to the test substance, the pads were removed and the test sites gently wiped with ethanol, water and a clean towel to remove any residual test substance.

Bodyweights: Individual bodyweights of the animals were recorded prior to test substance application (initial) and again on Days 7 and 14 (termination) (See Table 49).

### Cage-Side Observations:

The animals were observed for mortality, signs of gross toxicity, and behavioral changes at 1 and 5 hours after application and at least once daily thereafter for 14 days. Observations included gross evaluation of skin and fur, eyes and mucous membranes, respiratory, circulatory, autonomic and central nervous systems, somatomotor activity and behavior pattern. Particular attention was directed to observation of tremors, convulsions, salivation, diarrhea and coma (See Table 50).

Necropsy: All rats were euthanized via CO₂ inhalation on Day 14. Gross necropsies were performed on all animals. Tissues and organs of the thoracic and abdominal cavities were examined (See Table 51).

RESULTS: All animals survived, gained weight and appeared active and healthy. There were no signs of gross toxicity, adverse pharmacologic effects or abnormal behavior. No gross abnormalities were noted for the animals when necropsied at the conclusion of the 14-day observation period.

CONCLUSION: The single dose acute dermal LD₅₀ of ASC-1c is greater than 2,000 mg/kg of bodyweight.

TABLE 49
Individual Bodyweights And Doses

| Animal | | Bodyweight (g) | | | Dose |
|---|---|---|---|---|---|
| No. | Sex | Initial | Day 7 | Day 14 | mL |
| 41 | M | 227 | 285 | 310 | 0.52 |
| 42 | M | 256 | 302 | 324 | 0.58 |
| 43 | M | 225 | 277 | 305 | 0.51 |
| 44 | M | 273 | 320 | 342 | 0.62 |
| 45 | M | 287 | 332 | 358 | 0.65 |
| 46 | F | 205 | 265 | 324 | 0.47 |
| 47 | F | 221 | 262 | 308 | 0.50 |
| 48 | F | 222 | 253 | 295 | 0.51 |
| 49 | F | 233 | 267 | 303 | 0.53 |
| 50 | F | 223 | 250 | 300 | 0.51 |

TABLE 50
Individual Cage-Side Observations

| Animal | | Day of |
|---|---|---|
| Number | Findings | Occurrence |
| MALES: | | |
| 41-45 | Active and healthy | 0-14 |
| FEMALES: | | |
| 46-50 | Active and healthy | 0-14 |

TABLE 51
Individual Necropsy Observations

| Animal | | |
|---|---|---|
| Number | Tissue | Findings |
| MALES: | | |
| 41-45 | All tissues/organs | No gross abnormalities |
| FEMALES: | | |
| 46-50 | All tissues/organs | No gross abnormalities |

### Example 18

### Determination of Skin Irritation Caused by ASC-1 in Rabbits

This example demonstrates that the antiseptic compositions according to the invention do not cause skin irritation. The ability of an antiseptic composition according to the present invention to irritate skin was tested by exposing rabbits to a single topical exposure of ASC-1.

SUMMARY: A primary skin irritation test was conducted with rabbits to determine the potential for ASC-1 to produce irritation from a single topical application. Based on the results of this study, the test substance, ASC-1 was classified as non-irritating to the skin. Five-tenths mL of the test substance was applied to the skin of 3 healthy rabbits for 4 hours. Following exposure, dermal irritation was evaluated by the method of Draize et al. “Methods for the study of irritation and toxicity of substances applied topically to the skin and mucous membranes”. J. Pharmacol. Exp. Ther. 1944. No dermal irritation was noted at any dose site during the study. The Primary Dermal Irritation Index (PDH) calculated for this test substance was 0.0. The incidence and severity of irritation are summarized in Tables 52 and 53 below.

TABLE 52
Incidence of Irritation in response to treatment with ASC-1

| | |
|---|---|
| Time after | Incidence of Irritation |

| Patch Removal | Erythemea | Edema |
|---|---|---|
| 1 hour | 0/3 | 0/3 |
| 24 hours | 0/3 | 0/3 |
| 48 hours | 0/3 | 0/3 |
| 72 hours | 0/3 | 0/3 |

TABLE 53
Severity of Irritation in response to treatment with ASC-1

| Time after | Severity of |
|---|---|
| Patch Removal | Irritation - Mean Score |
| 1 hour | 0.0 |
| 24 hours | 0.0 |
| 48 hours | 0.0 |
| 72 hours | 0.0 |

### Materials:

Test Substance: The test substance was ASC-1, prepared as described in Example 1 and identified as ASC-1c. The test substance was a clear liquid, was stored at room temperature, and was not diluted prior to use. The test substance had a pH of 7.2 and was soluble in water and ethanol. The test substance was stable for the duration of testing.

Animals: There were 3 adult rabbits (2 male and 1 female), New Zealand albino strain, received from Davidson's Mill Farm, South Brunswick, N.J.

### Methods:

Preparation and Selection of Animals: On the day before application, a group of animals was prepared by clipping (Oster model #A5-small) the dorsal area and the trunk. After clipping and prior to application, the animals were examined for health and the skin checked for any abnormalities. Only healthy animals without pre-existing skin irritation were selected for test.

Application of Test Substance: Five-tenths of a milliliter of the test substance was applied to one 6 cm² intact dose site on each animal and covered with a 1 inch×1 inch, 4-ply gauze pad. The pad and entire trunk of each animal were then wrapped with semi-occlusive 3 inch Micropore tape to avoid dislocation of the pad. Elizabethan collars were placed on each rabbit and they were returned to their designated cages. After 4 hours of exposure to the test substance, the pads and collars were removed and the test sites gently wiped with ethanol, water and a clean towel to remove any residual test substance.

Evaluation of Test Sites: Individual dose sites were scored according to the Draize scoring system (Table 57) 1 at approximately 1, 24, 48 and 72 hours after patch removal. The classification of irritancy was obtained by adding the average erythema and edema scores for the 1, 24, 48 and 72 hour scoring intervals and dividing by the number of evaluation intervals (4).

The resulting Primary Dermal Irritation Index (PDII) was classified as shown in Table 54.

TABLE 54
Primary Dermal Irritation Index Classification

| PDII | Classification |
|---|---|
| <0.5 | Non-irritating |
| 0.5-2.0 | Slightly Irritating |
| 2.1-5.0 | Moderately Irritating |
| >5.0 | Severely Irritating |

Cage-Side Observations: The animals were observed for signs of gross toxicity and behavioral changes at least once daily during the test period. Observations included gross evaluation of skin and fur, eyes and mucous membranes, respiratory, circulatory, autonomic and central nervous systems, somatomotor activity and behavior pattern. Particular attention was directed to observation of tremors, convulsions, salivation, diarrhea and coma.

RESULTS: All animals appeared active and healthy. There were no signs of gross toxicity, adverse pharmacologic effects or abnormal behavior. No dermal irritation was noted at any dose site during the study. The individual scores for each animal for skin irritation are shown in Table 55. A summary of the primary skin irritation scores are shown in Table 57. The Primary Dermal Irritation Index for ASC-1c was 0.0.

CONCLUSION: Based on the classification system used, ASC-1c was classified as non-irritating to the skin.

TABLE 55
Individual Skin Irritation Scores For Erythema/Edema

| | |
|---|---|
| Animal | Hours after Patch Removal |

| No. | Sex | 1 | 24 | 48 | 72 |
|---|---|---|---|---|---|
| 9708 | F | 0/0 | 0/0 | 0/0 | 0/0 |
| 9709 | F | 0/0 | 0/0 | 0/0 | 0/0 |
| 9710 | M | 0/0 | 0/0 | 0/0 | 0/0 |

| | | | | |
|---|---|---|---|---|
| Total | 0/0 | 0/0 | 0/0 | 0/0 |
| Mean | 0.0/0.0 | 0.0/0.0 | 0.0/0.0 | 0.0/0.0 |

TABLE 56
Summary Of Primary Skin Irritation Scores

Hours after Patch Removal

| | 1 | 24 | 48 | 72 |
|---|---|---|---|---|
| Erythema | 0.0 | 0.0 | 0.0 | 0.0 |
| Edema | 0.0 | 0.0 | 0.0 | 0.0 |
| TOTAL (PDI) | 0.0 | 0.0 | 0.0 | 0.0 |

TABLE 57
Primary Skin Irritation Scoring System

| Evaluation of Skin Reactions | Value |
|---|---|
| Erythema and Eschar Formation: | |
| No erythema | 0 |
| Very slight erythema (barely perceivable) | 1 |
| Well-defined erythema | 2 |
| Moderate to severe erythema | 3 |
| Severe erythema (beet redness) to slight | 4 |
| eschar formation (injuries in depth) | |
| Edema Formation: | |
| No edema | 0 |
| Very slight edema (barely perceptible) | 1 |
| Slight edema (edges of area well defined by | 2 |
| definite raising) | |
| Moderate edema (raised approximately 1 mm) | 3 |
| Severe edema (raised more than 1 mm and | 4 |
| extending beyond the area of exposure) | |

### Example 19

### Determination of Primary Eye Irritation in Rabbits Exposed to ASC-1

This example demonstrates that the antiseptic compositions of the invention do not cause eye irritation. The ability of an antiseptic composition according to the invention to irritate eyes was tested by exposing rabbits to a single instillation of ASC-1 via the ocular route.

SUMMARY: A primary eye irritation test was conducted with rabbits to determine the potential for ASC-1 to produce irritation. Based on the results of this study, the test substance is classified as moderately irritating to the unrinsed eye and severely irritating to the rinsed eye.

One-tenth of a milliliter of the test substance was instilled into the right eye of six healthy rabbits. The treated eyes of three rabbits were rinsed with physiological saline after instillation. The eyes of the remaining three rabbits were not rinsed. The left eye remained untreated and served as a control. Ocular irritation was evaluated by the method of Draize et al. “Methods for the study of irritation and toxicity of substances applied topically to the skin and mucous membranes”. J. Pharmacol. Exp. Ther. 1944.

Within 24 hours of test substance instillation, corneal opacity, iritis and conjunctivitis were observed in all six treated eyes (3 unrinsed and 3 rinsed). Pannus was also evident in one unrinsed eye and one rinsed eye, between Days 7 and 17. The incidence and/or severity of irritation decreased with time. Although all unrinsed eyes and two rinsed eyes were free of irritation by study termination (Day 21), corneal opacity persisted in one rinsed eye through Day 21 (termination).

The incidence, severity and reversibility (if applicable) of irritation are summarized below in Tables 58 and 59.

TABLE 58
Summary of Incidence of Irritation from exposure to ASC-1

| | Incidence of Irritation | | | | | |
|---|---|---|---|---|---|---|
| | Unrinsed | | | Rinsed | | |
| Time Post | Corneal | | Con- | Corneal | | Con- |
| Instillation | Opacity | Iritis | junctivitis | Opacity | Iritis | junctivitis |
| 1 hour | 0/3 | 0/3 | 3/3 | 0/3 | 1/3 | 3/3 |
| 24 hours | 3/3 | 3/3 | 3/3 | 3/3 | 3/3 | 3/3 |
| 48 hours | 3/3 | 2/3 | 3/3 | 3/3 | 2 | 3/3 |
| 72 hours | 3/3 | 2/3 | 3/3 | 3/3 | 3 | 3/3 |
| Day 4 | 3/3 | 2/3 | 3/3 | 3/3 | 1/3 | 3/3 |
| Day 7 | 3/3 | 0/3 | 2/3 | 2/3 | 1/3 | 2/3 |
| Day 10 | 3/3 | 0/3 | 2/3 | 2/3 | 1/3 | 1/3 |
| Day 14 | 3/3 | 0/3 | 0/3 | 0/3 | 0/3 | 1/3 |
| Day 17 | 3/3 | 0/3 | 0/3 | 0/3 | 0/3 | 1/3 |
| Day 21 | 3/3 | 0/3 | 0/3 | 0/3 | 0/3 | 0/3 |

TABLE 59
Summary of Severity of Irritation from exposure to ASC-1

| | |
|---|---|
| Time Post | Severity of Irritation - Mean Score |

| Instillation | Unrinsed | Rinsed |
|---|---|---|
| 1 hour | 7.3 | 9.7 |
| 24 hours | 29.0 | 27.3 |
| 48 hours | 23.0 | 23.0 |
| 72 hours | 19.0 | 18.3 |
| Day 4 | 19.0 | 16.0 |
| Day 7 | 5.0 | 13.0 |
| Day 10 | 4.3 | 12.3 |
| Day 14 | 0.0 | 3.0 |
| Day 17 | 0.0 | 2.3 |
| Day 21 | 0.0 | 1.7 |

### Materials:

Test Substance The test substance was ASC-1, prepared as described in Example 1 and identified as ASC-1c. The test substance was a clear liquid, was stored at room temperature, and was not diluted prior to use. The test substance had a pH of 7.2 and was soluble in water and ethanol. The test substance was stable for the duration of testing.

Animals: There were 6 adult rabbits (4 male and 2 female), New Zealand albino strain,

### Methods:

Preparation and Selection of Animals: Prior to instillation, both eyes of a number of animals were examined using a fluorescein dye procedure. One drop of 2% ophthalmic fluorescein sodium was instilled into both eyes of each of the rabbits. The eyes were rinsed with physiological saline (0.9% NaCl) approximately 30 seconds after instillation of the fluorescein. Using a Blak-Ray® Lamp (compact 4 watt UV Lamp), the eyes were checked for gross abnormalities according to the “Scale for Scoring Ocular Lesions” (Table 3). Only healthy animals without pre-existing ocular irritation were selected for test.

Instillation: One-tenth of a milliliter of the test substance was instilled into the conjunctival sac of the right eye of each rabbit by pulling the lower lid away from the eyeball. The upper and lower lids were then gently held together for about one second before releasing, to minimize loss of the test substance. The other eye of each rabbit remained untreated with the test substance and served as a control. The treated eyes of three rabbits were rinsed with approximately 30 milliliters of physiological saline (0.9% NaCl) approximately 20-30 seconds after instillation of the test substance. The eyes of the remaining three animals were not rinsed. The rabbits were then returned to their designated cages.

Ocular Scoring: Ocular irritation was evaluated using a high-intensity white light (Mag Lite) in accordance with Draize et al. (Schedule 1) at 1, 24, 48 and 72 hours and at 4, 7, 10, 14, 17 and 21 days post-instillation. The fluorescein dye evaluation procedure described above was used at 24 hours and as needed at subsequent scoring intervals to evaluate the extent of corneal damage or to verify reversal of effects. Individual scores were recorded for each animal. In addition to observations of the cornea, iris and conjunctivae, any other observed lesions were noted. The average score for all rabbits at each scoring period was calculated to aid in data interpretation.

Classification of Eye Scores: The time interval with the highest mean score (Maximum Mean Total Score—MMTS) for each group of rabbits (unrinsed and rinsed eyes) was used to further classify the test substance (Table 65) as described in Kay, J. H. et al. Interpretation of eye irritation tests. J. Soc. Cos. Chem. 1962; 13:281-289.

Cage-Side Observations: The animals were observed for signs of gross toxicity and behavioral changes at least once daily during the test period. Observations included gross evaluation of skin and fur, eyes and mucous membranes, respiratory, circulatory, autonomic and central nervous systems, somatomotor activity and behavior pattern. Particular attention was directed to observation of tremors, convulsions, salivation, diarrhea and coma.

RESULTS: Individual ocular irritation scores for the animals tested are shown in Tables 60-65. All animals appeared active and healthy. Apart from the eye irritation noted below, there were no other signs of gross toxicity, adverse pharmacologic effects or abnormal behavior. Within 24 hours of test substance instillation, corneal opacity, iritis and conjunctivitis were observed in all six treated eyes (3 unrinsed and 3 rinsed). Pannus was also evident in one unrinsed eye and one rinsed eye between Days 7 and 17. The incidence and/or severity of irritation decreased with time. Although all unrinsed eyes and two rinsed eyes were free of irritation by study termination (Day 21), corneal opacity persisted in one rinsed eye through Day 21 (termination). The Maximum Mean Total Score of ASC-1 was 29.0 and 27.3 for the unrinsed and rinsed eye, respectively.

CONCLUSION: Based on the classification system used, ASC-1 was classified as moderately irritating to the rinsed eye.

TABLE 60
Individual Scores for Ocular Irritation - Unrinsed Eyes

| | Rabbit No. 9787 (Male) | | | | | | | | | |
|---|---|---|---|---|---|---|---|---|---|---|
| | Hours | | | | Days | | | | | |
| | 1 | 24 | 48 | 72 | 4 | 7 | 10 | 14 | 17 | 21 |
| Cornea | | | | | | | | | | |
| A: Opacity | 0 | 1¹ | 1 | 1¹ | 1 | 1^{1,2} | 1² | 0¹ | 0 | 0 |
| B: Area | 4 | 3 | 3 | 2 | 2 | 1 | 1 | 4 | 4 | 4 |
| (A × B) × 5 | 0 | 15 | 15 | 10 | 10 | 5 | 5 | 0 | 0 | 0 |
| Iris | | | | | | | | | | |
| A: Values | 0 | 1 | 1 | 1 | 1 | 0 | 0 | 0 | 0 | 0 |
| A × 5 | 0 | 5 | 5 | 5 | 5 | 0 | 0 | 0 | 0 | 0 |
| Conjunctivae | | | | | | | | | | |
| A: Redness | 1 | 2 | 2 | 2 | 2 | 1 | 1 | 0 | 0 | 0 |
| B: Chemosis | 1 | 1 | 1 | 1 | 1 | 1 | 1 | 0 | 0 | 0 |
| C: Discharge | 2 | 2 | 2 | 2 | 2 | 1 | 1 | 0 | 0 | 0 |
| (A + B + C) × | 8 | 10 | 10 | 10 | 10 | 6 | 6 | 0 | 0 | 0 |
| 2 | | | | | | | | | | |
| TOTAL | 8 | 30 | 30 | 25 | 25 | 11 | 11 | 0 | 0 | 0 |

¹2% fluorescein sodium used to evaluate the extent or verify the absence of corneal opacity.
²Pannus Present.

TABLE 61
Individual Scores For Ocular Irritation - Unrinsed Eyes

| | Rabbit No. 9788 (Female) | | | | | | | | | |
|---|---|---|---|---|---|---|---|---|---|---|
| | Hours | | | | Days | | | | | |
| | 1 | 24 | 48 | 72 | 4 | 7 | 10 | 14 | 17 | 21 |
| Cornea | | | | | | | | | | |
| A: Opacity | 0 | 1¹ | 1 | 1¹ | 1¹ | 0¹ | 0 | 0 | 0 | 0 |
| B: Area | 4 | 2 | 1 | 1 | 1 | 4 | 4 | 4 | 4 | 4 |
| (A × B) × 5 | 0 | 10 | 5 | 5 | 5 | 0 | 0 | 0 | 0 | 0 |
| Iris | | | | | | | | | | |
| A: Values | 0 | 1 | 0 | 0 | 0 | 0 | 0 | 0 | 0 | 0 |
| A × 5 | 0 | 5 | 0 | 0 | 0 | 0 | 0 | 0 | 0 | 0 |
| Conjunctivae | | | | | | | | | | |
| A: Redness | 1 | 2 | 0 | 0 | 0 | 0 | 0 | 0 | 0 | 0 |
| B: Chemosis | 1 | 1 | 0 | 0 | 0 | 0 | 0 | 0 | 0 | 0 |
| C: Discharge | 2 | 2 | 1 | 1 | 1 | 0 | 0 | 0 | 0 | 0 |
| (A + B + C) × 2 | 8 | 10 | 2 | 2 | 2 | 0 | 0 | 0 | 0 | 0 |
| TOTAL | 8 | 25 | 7 | 7 | 7 | 0 | 0 | 0 | 0 | 0 |

¹2% fluorescein sodium used to evaluate the extent or verify the absence of corneal opacity.
²Pannus Present.

TABLE 62
Individual Scores for Ocular Irritation - Unrinsed Eyes

| | Rabbit No. 9789 (Male) | | | | | | | | | |
|---|---|---|---|---|---|---|---|---|---|---|
| | Hours | | | | Days | | | | | |
| | 1 | 24 | 48 | 72 | 4 | 7 | 10 | 14 | 17 | 21 |
| Cornea | | | | | | | | | | |
| A: Opacity | 0 | 1¹ | 1 | 1¹ | 1 | 0¹ | 0 | 0 | 0 | 0 |
| B: Area | 4 | 3 | 3 | 2 | 2 | 4 | 4 | 4 | 4 | 4 |
| (A × B) × 5 | 0 | 15 | 15 | 10 | 10 | 0 | 0 | 0 | 0 | 0 |
| Iris | | | | | | | | | | |
| A: Values | 0 | 1 | 1 | 1 | 1 | 0 | 0 | 0 | 0 | 0 |
| A × 5 | 0 | 5 | 5 | 5 | 5 | 0 | 0 | 0 | 0 | 0 |
| Conjunctivae | | | | | | | | | | |
| A: Redness | 1 | 3 | 3 | 2 | 2 | 1 | 0 | 0 | 0 | 0 |
| B: Chemosis | 1 | 1 | 1 | 1 | 1 | 0 | 0 | 0 | 0 | 0 |
| C: Discharge | 1 | 2 | 2 | 2 | 2 | 1 | 1 | 0 | 0 | 0 |
| (A + B + C) × 2 | 6 | 12 | 12 | 10 | 10 | 4 | 2 | 0 | 0 | 0 |
| TOTAL | 6 | 32 | 32 | 25 | 25 | 40 | 2 | 0 | 0 | 0 |

¹2% fluorescein sodium used to evaluate the extent or verify the absence of corneal opacity.
²Pannus Present.

TABLE 63
Individual Scores For Ocular Irritation - Rinsed Eyes

| | Rabbit No. 9790 (Male) | | | | | | | | | |
|---|---|---|---|---|---|---|---|---|---|---|
| | Hours | | | | Days | | | | | |
| | 1 | 24 | 48 | 72 | 4 | 7 | 10 | 14 | 17 | 21 |
| Cornea | | | | | | | | | | |
| A: Opacity | 0 | 1¹ | 1 | 1¹ | 0¹ | 0 | 0 | 0 | 0 | 0 |
| B: Area | 4 | 1 | 1 | 1 | 4 | 4 | 4 | 4 | 4 | 4 |
| (A × B) × 5 | 0 | 5 | 5 | 5 | 0 | 0 | 0 | 0 | 0 | 0 |
| Iris | | | | | | | | | | |
| A: Values | 1 | 1 | 0 | 0 | 0 | 0 | 0 | 0 | 0 | 0 |
| A × 5 | 5 | 5 | 0 | 0 | 0 | 0 | 0 | 0 | 0 | 0 |
| Conjunctivae | | | | | | | | | | |
| A: Redness | 1 | 2 | 1 | 0 | 0 | 0 | 0 | 0 | 0 | 0 |
| B: Chemosis | 1 | 1 | 0 | 0 | 0 | 0 | 0 | 0 | 0 | 0 |
| C: Discharge | 2 | 2 | 1 | 1 | 1 | 0 | 0 | 0 | 0 | 0 |
| (A + B + C) × 2 | 8 | 10 | 4 | 2 | 2 | 0 | 0 | 0 | 0 | 0 |
| TOTAL | 13 | 20 | 9 | 7 | 2 | 0 | 0 | 0 | 0 | 0 |

¹2% fluorescein sodium used to evaluate the extent or verify the absence of corneal opacity.
²Pannus Present.

TABLE 64
Individual Scores For Ocular Irritation - Rinsed Eyes

| | Rabbit No. 9791 Female) | | | | | | | | | |
|---|---|---|---|---|---|---|---|---|---|---|
| | Hours | | | | Days | | | | | |
| | 1 | 24 | 48 | 72 | 4 | 7 | 10 | 14 | 17 | 21 |
| Cornea | | | | | | | | | | |
| A: Opacity | 0 | 2¹ | 2 | 2 | 2¹ | 2^{1,2} | 2² | 1^{1,2} | 1^{1,2} | 1¹ |
| B: Area | 4 | 2 | 2 | 2 | 2 | 2 | 2 | 1 | 1 | 1 |
| (A × B) × 5 | 0 | 20 | 20 | 20 | 20 | 20 | 20 | 5 | 5 | 05 |
| Iris | | | | | | | | | | |
| A: Values | 0 | 1 | 1 | 1 | 1 | 1 | 0 | 0 | 0 | 0 |
| A × 5 | 0 | 5 | 5 | 5 | 5 | 5 | 5 | 0 | 0 | 0 |
| Conjunctivae | | | | | | | | | | |
| A: Redness | 1 | 2 | 2 | 2 | 2 | 2 | 2 | 0 | 0 | 0 |
| B: Chemosis | 1 | 1 | 1 | 2 | 2 | 2 | 2 | 1 | 0 | 0 |
| C: Discharge | 2 | 2 | 2 | 2 | 2 | 2 | 2 | 1 | 1 | 0 |
| (A + B + C) × 2 | 8 | 10 | 10 | 12 | 12 | 12 | 2 | 4 | 2 | 0 |
| TOTAL | 8 | 35 | 35 | 37 | 37 | 37 | 37 | 9 | 7 | 5 |

¹2% fluorescein sodium used to evaluate the extent or verify the absence of corneal opacity.
²Pannus Present.

TABLE 65
Individual Scores for Ocular Irritation - Rinsed Eyes

| | Rabbit No. 9792 (Male) | | | | | | | | | |
|---|---|---|---|---|---|---|---|---|---|---|
| | Hours | | | | Days | | | | | |
| | 1 | 24 | 48 | 72 | 4 | 7 | 10 | 14 | 17 | 21 |
| Cornea | | | | | | | | | | |
| A: Opacity | 0 | 1¹ | 1 | 1 | 1¹ | 0¹ | 0 | 0 | 0 | 0 |
| B: Area | 4 | 2 | 2 | 1 | 1 | 4 | 4 | 4 | 4 | 4 |
| (A × B) × 5 | 0 | 10 | 10 | 5 | 5 | 0 | 0 | 0 | 0 | 0 |
| Iris | | | | | | | | | | |
| A: Values | 0 | 1 | 1 | 0 | 0 | 0 | 0 | 0 | 0 | 0 |
| A × 5 | 0 | 5 | 5 | 0 | 0 | 0 | 0 | 0 | 0 | 0 |
| Conjunctivae | | | | | | | | | | |
| A: Redness | 1 | 3 | 2 | 1 | 1 | 0 | 0 | 0 | 0 | 0 |
| B: Chemosis | 1 | 1 | 1 | 1 | 0 | 0 | 0 | 0 | 0 | 0 |
| C: Discharge | 2 | 2 | 2 | 1 | 1 | 1 | 0 | 0 | 0 | 0 |
| (A + B + C) × 2 | 8 | 12 | 10 | 6 | 4 | 2 | 0 | 0 | 0 | 0 |
| TOTAL | 8 | 27 | 25 | 11 | 9 | 2 | 0 | 0 | 0 | 0 |

¹2% fluorescein sodium used to evaluate the extent or verify the absence of corneal opacity.
²Pannus Present.

TABLE 66
Classification of Eye Irritation Scores

| | Irritation | |
|---|---|---|
| MMTS | Classification | Requirement for Maintenance of Classification¹ |
| 0.0-0.5 | Non | Up to 0.5 at 1 hour, with zeros at 24 hours; otherwise increase |
| | | one level. |
| 0.6-2.5 | Practically non | With zeros at 48 hours; otherwise, increase one level. |
| 2.6-15.0 | Minimally | With zeros at 48 hours; otherwise increase one level. |
| 15.1-25.0 | Mildly | With zeros at 96 hours; otherwise increase one level. |
| 25.1-50.0 | Moderately | With 7 day mean ≦20 and individual total scores ≦10 in at least |
| | | 60% of the rabbits with no total score >30; otherwise increase |
| | | one level. |
| 50.1-80.0 | Severely | With 7 day mean ≦40 and individual total scores ≦30 in at least |
| | | 60% of the rabbits with no total score >60; otherwise increase |
| | | one level. |
| 80.1-100.0 | Extremely | With 7 day mean ≦80 and individual total scores ≦60 in at least |
| | | 60% of the rabbits with no total score >100; otherwise increase |
| | | one level. |
| 100.1-110.0 | Maximally | With 7 day mean ≦80 and individual total scores >60 in at least |
| | | 60% of the rabbits with no total score >100; otherwise decrease |
| | | one level. |

¹Kay J H, and Calandra J C. Interpretation of eye irritation tests. J Soc Cos Chem 1962; 13: 281-289.

Schedule 1: Scale For Scoring Ocular Lesions¹

| | |
|---|---|
| 1. CORNEA | |
| A: Opacity-degree of density (area most dense taken for reading) | |
| Opacity. | 0 |
| Scattered or diffuse area, details of iris clearly visible | 1* |
| Easily discernible translucent areas, details of iris slightly obscured | 2* |
| Opalescent areas, no details of iris visible, size of pupil barely discernible. | 3* |
| Opaque, iris invisible | 4* |
| B. Area of cornea involved | |
| One quarter (or less) but not zero | 1 |
| Greater than one quarter, but less than half | 2 |
| Greater than half, but less than three quarters | 3 |
| Greater than three quarters, up to whole area | 4 |
| A × B × 5 Total Maximum = 80 | |
| 2. IRIS | |
| A. Values | |
| Normal | 0 |
| Folds above normal, congestion, swelling, circumcorneal injection (any or all of | 1* |
| these or combination of any thereof) iris still reacting to light (sluggish reaction is | |
| positive) | |
| No reaction to light, hemorrhage, gross destruction (any or all of these) | 2* |
| A × 5 Total Maximum = 10 | |
| 3. Conjunctivae | |
| A. Redness (refers to palpebral and bulbar conjunctivae excluding cornea and iris) | |
| Vessels normal | 0 |
| Vessels definitely injected above normal | 1 |
| More diffuse, deeper crimson red, individual vessels not easily discernible | 2* |
| Diffuse beefy red | 3* |
| B. Chemosis | |
| No swelling | 0 |
| Any swelling above normal (includes nictitating membrane) | 1 |
| Obvious swelling with partial eversion of lids | 2* |
| Swelling with lids about half-closed | 3* |
| Swelling with lids about half-closed to completely closed | 4* |
| C. Discharge | |
| No discharge | 0 |
| Any amount different from normal (does not include small amounts observed in | 1 |
| inner | |
| canthus of normal animals) | |
| Discharge with moistening of the lids and hairs just adjacent to lids | 2 |
| Discharge with moistening of the lids and hairs, and considerable area around the | 3 |
| eye | |
| Score (A + B + C) × 2 Total Maximum = 20 | |
| Total Maximum Score: 110 represents the sum of all scores obtained for the cornea, | |
| iris and conjunctivae. | |

*These scores represent a positive response.
¹Draize, J. H., Woodward, G. and Calvery, H. O. Methods for the study of irritation and toxicity of substances applied topically to the skin and mucous membranes. J. Pharmacol. Exp. Ther. 1944; 82: 377-390.

### Example 20

### Determination of Skin Sensitization Reactions Caused by ASC-1 in Guinea Pigs

The following example demonstrates that the antiseptic compositions according to the invention do not cause sensitization in skin. The potential for an antiseptic composition according to the invention to elicit a skin sensitization reaction was determined by conducting a dermal sensitization test with ASC-1 as described following.

SUMMARY: A dermal sensitization test was conducted with guinea pigs to determine the potential for ASC-1 (test substance) to produce sensitization after repeated topical applications. The test substance was topically applied to 20 healthy test guinea pigs, once each week for a 3 week induction period. Then 27 days after the first induction dose, a challenge dose of the test substance at its highest non-irritating concentration (100%) was applied to a naive site on each guinea pig. A naive control group (10 animals) was maintained under the same environmental conditions and treated with the test substance at challenge only. Approximately 24 and 48 hours after each induction and challenge dose, the animals were scored for erythema. A summary of the sensitization response noted after challenge is described in Table 67.

TABLE 67
Summary Of Incidence And Severity Of Sensitization Response

| Sensitization Response Indices | |
|---|---|
| Incidence of | |
| Positive Responses¹ | Severity² |
| Hours | Hours |

| | 24 | 48 | 24 | 48 |
|---|---|---|---|---|
| Test Animals | 0/20 | 0/20 | 0.25 | 0.18 |
| Test Naïve | 0/10 | 0.10 | 0.25 | 0.10 |
| Control Animals | | | | |

¹Animals with scores greater than 0.5.
²Sum of the erythema scores divided by the number of animals evaluated.

Based on the results of this study, the test substance is not considered to be a contact sensitizer. Historical data indicating a positive response to 0.04% DNCB (dinitrochlorobenzene) in acetone validates the test system used in this study.

### Materials:

Test Substance: The test substance was ASC-1, prepared as described in Example 1 and identified as ASC-1c The test substance was a clear liquid, was stored at room temperature, and was not diluted prior to use. The test substance had a pH of 7.2 and was soluble in water and ethanol. The test substance was stable for the duration of testing.

### Animals:

### Number of Animals: 34

### Number of Groups: 3

### Number of Animals per Group:

Preliminary Irritation Group: 4
Test Group: 20
Test Naive Control Group: 10

### Sex: 20 Male and 14 Female

Species/Strain: Guinea pigs/Hartley albino

### Age/Bodyweight:

Preliminary Irritation Group: Young adult
Test Group: Young adult/males 345-381 grams and females 329-378 grams at experimental start
Source: Received from Davidson's Mill Farms, South Brunswick, N.J.

### Methods:

Preliminary Irritation Testing: A group of animals was used to determine the highest non-irritating concentration (HNIC) of test substance prior to the challenge dose. The fur was removed by clipping (Oster model #A2-small) the dorsal area and flanks of each guinea pig. This area was divided into four test sites (two sites on each side of the midline) on each animal. The test substance was applied neat (100%) and also diluted with distilled water to yield concentrations of 75%, 50% and 25% w/w. Each concentration was applied to a test site using an occlusive 25 mm Hill Top Chamber®. The sites were wrapped with non-allergenic Durapore adhesive tape.

After 6 hours of exposure, the chambers were removed and the dose sites were gently wiped with water and a clean towel to remove any residual test substance.

Twenty-four hours after application, each site was evaluated for local reactions (erythema) according to the scoring system described in the section entitled “Scoring System” as follows. From these results, the HNIC (the highest concentration that produced responses in 4 guinea pigs no more severe than two scores of 0.5 and two scores of zero) was established and used for challenge. Based on these findings (See Tables 68 and 69), the HNIC selected for the challenge phase was 100%.

Preparation and Selection of Animals: On the day before initiation, the fur of a group of animals was removed by clipping the dorsal area and flanks. After clipping and prior to initiation, the animals were weighed and the skin was checked for any abnormalities. Only healthy animals without pre-existing skin irritation were selected for test. Animals were reclipped prior to each dose.

Induction Phase: Once each week for 3 weeks, four-tenths of a milliliter of the test substance was applied, as received, to the left side of each test animal using an occlusive 25 mm Hill Top Chamber®. The chambers were secured in place and wrapped with non-allergenic Durapore adhesive tape to avoid dislocation of the chambers and to minimize loss of the test substance. After the 6-hour exposure period, the chambers were removed and the test sites were gently wiped with water and a clean towel to remove any residual test substance. Then 24 and 48 hours after each induction application, readings were made of local reactions (erythema) according to the scoring system described in the section entitled “Scoring System” as follows. (See Table 73).

Challenge Phase: 27 days after the first induction dose, four-tenths of a milliliter of the HNIC of the test substance (100%) was applied to a naive site on the right side of each animal as a challenge dose, using the procedures described above. These sites were evaluated for a sensitization response (erythema) at 24 and 48 hours after the challenge application according to the system described in the section entitled “Scoring System” as follows (See Tables 75 and 76).

In addition to the test animals, 10 guinea pigs from the same shipment were maintained under identical environmental conditions and were treated with the HNIC of the test substance at challenge only. These animals constituted the “naive” group.

### Scoring System:

0—no reaction
0.5—very faint erythema, usually non-confluent
1—faint erythema usually confluent
b 2—moderate erythema
3—severe erythema with or without edema

Bodyweights: Individual bodyweights of the animals were recorded prior to initiation and again on the day after challenge (See Tables 70 and 71).

EVALUATION: In order to evaluate the sensitization response, two indices were used; one for incidence and one for severity in both test and positive control animals. The incidence index was calculated to evaluate the incidence of erythema (sensitization response) 24 and 48 hours after challenge according to the following formula:

$$Number of animals evaluated$$

$$Incidence Index = \frac{Number of erythema scores > 0.5}{Number of animals evaluated}$$

The severity index (sensitization produced) at 24 and 48 hours after challenge was calculated using the following formula:

$$Number of animals evaluated$$

$$Severity Index = \frac{Sum of erythema scores}{Number of animals evaluated}$$

The following criteria were used to classify the test substance as a potential contact sensitizer (Ritz & Buehler, 1980): At the 24 hour and/or 48 hour scoring interval, 15% or more of the test animals exhibit a positive response (scores >0.5) in the absence of similar results in the naive control group. The positive reaction must persist to 48 hours in at least one test animal.

VALIDATION—POSITIVE CONTROL: The procedures used in this study were validated using 1-Chloro-2,4-Dinitrobenzene (DNCB), as a positive control substance. Prior to carrying out studies with the test substances, a Buehler sensitization test was conducted with DNCB using Hartley strain albino guinea pigs from Davidson's Mills Farm following induction and challenge procedures identical to those described above. The results obtained from this testing are presented in Tables 68, 69, 72, 74, and 77.

### Results:

### Induction Phase:

Test Animals (ASC-1c applied): Very faint to moderate erythema (0.5-2) was observed at all test sites during the induction phase. Overall, the incidence and/or severity of irritation increased with each successive application, with desquamation evident at one test site 48 hours after the second induction.

Historical Positive Control Animals (0.08% DNCB in 80% aqueous ethanol): Very faint to severe erythema (0.5-3) was observed at all positive control sites during the induction phase. Overall, the incidence and severity of irritation increased with each successive application.

### Challenge Phase:

Test Animals (ASC-1c applied): Very faint erythema (0.5) was observed at ten test sites 24 hours after challenge. Irritation persisted at seven of these sites through 48 hours.

Test Naive Control Animals (ASC-1c applied): Very faint erythema (0.5) was observed at 5 test sites 24 hours after challenge. Irritation persisted at 2 of these sites through 48 hours.

Historical Positive Control Animals (0.04% DNCB in acetone): 8 of 10 positive control animals exhibited signs of a sensitization response (faint erythema [1-2]) 24 and/or 48 hours after challenge.

Historical Positive Naive Control Animals (0.04% DNCB in acetone): Very faint erythema (0.5) was observed at two positive naive control sites 24 hours after challenge. Irritation cleared from both affected sites within 48 hours.

CONCLUSION: Based on these findings and on the evaluation system used, ASC-1 is not considered to be a contact sensitizer. Historical data indicating a positive response to 0.04% DNCB in acetone validates the test system used in this study.

TABLE 68
Preliminary Irritation Testing Scores For
Determination Of HNIC¹ for Test Substance

| | |
|---|---|
| Animal | Concentration (%) |

| No &/Sex | [symbol] 0 | 75² | 50² | 25² |
|---|---|---|---|---|
| 2017/F | | 0 | 0 | 0 |
| 2017/F | | 0 | 0 | 0 |
| 2017/F | | 0 | 0 | 0 |
| 2017/F | | 0 | 0 | 0 |

[symbol]

indicates data missing or illegible when filed

TABLE 69
Preliminary Irritation Testing Scores For Determination Of
HNIC¹ for Positive Control - DNCB (Historical Data)

Concentration (%)³

| Animal No &/Sex | [symbol] 05 | 0.04 | 0.03 | 0.02 |
|---|---|---|---|---|
| 9676/M | 5 | 0.5 | 0 | 0 |
| 9677/M | 5 | 0 | 0 | 0 |
| 9678/M | 5 | 0.5 | 0.5 | 0 |
| 9679/M | 5 | 0 | 0 | 0 |

¹HNIC (Highest Non-Irritating Concentration)
²Four-tenths mL of the test substance was applied as w/w solutions in distilled water.
³Four-tenths mL of DNCB was applied as w/w solutions in acetone. [symbol]
indicates data missing or illegible when filed

TABLE 70
Individual Bodyweights (Test Substance, Test Animals)

| | | | Day After |
|---|---|---|---|
| Animal No. | | Initial | Challenge |
| TEST | Sex | (g) | (g) |
| 2018 | F | 378 | 479 |
| 2019 | F | 349 | 522 |
| 2020 | F | 329 | 458 |
| 2021 | M | 356 | 553 |
| 2022 | M | 346 | 454 |
| 2023 | M | 351 | 527 |
| 2024 | F | 349 | 472 |
| 2025 | F | 360 | 422 |
| 2026 | M | 355 | 522 |
| 2027 | M | 352 | 478 |
| 2028 | F | 341 | 464 |
| 2029 | F | 369 | 520 |
| 2030 | F | 360 | 528 |
| 2031 | M | 379 | 469 |
| 2032 | M | 352 | 547 |
| 2033 | M | 345 | 509 |
| 2034 | F | 361 | 485 |
| 2035 | F | 359 | 465 |
| 2036 | M | 362 | 549 |
| 2037 | M | 381 | 500 |

TABLE 71
Individual Bodyweights (Naïve Control, Test Animals)

| Animal No. | | | Day After |
|---|---|---|---|
| NAÏVE | | Initial | Challenge |
| CONTROL | Sex | (g) | (g) |
| 2038 | M | 371 | 482 |
| 2039 | M | 360 | 395 |
| 2040 | M | 358 | 496 |
| 2041 | M | 362 | 443 |
| 2042 | M | 347 | 545 |
| 2043 | M | 354 | 473 |
| 2044 | M | 349 | 500 |
| 2045 | M | 362 | 500 |
| 2046 | M | 358 | 474 |
| 2047 | M | 347 | 492 |

TABLE 72
Individual Bodyweights (Positive Control - DNCB, Historical
Data)

| Animal No. | | | Day After |
|---|---|---|---|
| POSITIVE | | Initial | Challenge |
| CONTROL | Sex | (g) | (g) |
| 781 | F | 428 | 544 |
| 782 | F | 372 | 609 |
| 783 | F | 393 | 568 |
| 784 | M | 406 | 579 |
| 785 | M | 379 | 544 |
| 786 | M | 413 | 600 |
| 787 | F | 404 | 660 |
| 788 | F | 415 | 581 |
| 789 | M | 406 | 618 |
| 790 | M | 423 | 668 |
| 791 | F | 411 | 536 |
| 792 | F | 408 | 591 |
| 793 | M | 408 | 551 |
| 794 | M | 386 | 639 |
| 795 | M | 407 | 599 |

TABLE 73
Induction Phase Skin Reaction Scores (Test Substance)

| Induction Number | | |
|---|---|---|
| 1 | 2 | 3 |
| Concentration¹ | | |
| 100% | 100% | 100% |
| Amount Applied (mL) | | |
| 0.4 | 0.4 | 0.4 |

Hours²

| | 24 | 48 | 24 | 48 | 24 | 48 |
|---|---|---|---|---|---|---|
| Animal No. | | | | | | |
| 2018 | 0 | 0 | 0.5 | 0.5 | 1 | 1 |
| 2019 | 0 | 0 | 1 | 1 | 2 | 2 |
| 2020 | 0.5 | 0 | 1 | 1 | 0.5 | 0 |
| 2021 | 1 | 0.5 | 1 | 1 | 1 | 1 |
| 2022 | 0 | 0 | 0 | 0 | 0.5 | 0 |
| 2023 | 0 | 0 | 1 | 1 | 2 | 2 |
| 2024 | 0 | 0 | 0 | 1 | 0 | 0 |
| 2025 | 0 | 0 | 0.5 | 0 | 0 | 0 |
| 2026 | 0 | 0.5 | 0.5 | 0.5 | 2 | 2 |
| 2027 | 0 | 0.5 | 1 | 1 | 1 | 0.5 |
| 2028 | 0.5 | 0.5 | 1 | 0.5 | 1 | 0.5 |
| 2029 | 0 | 0 | 0.5 | 0.5 | 0.5 | 0 |
| 2030 | 0 | 0 | 0.5 | 0 | 1 | 0.5 |
| 2031 | 0 | 0.5 | 1 | 0.5 | 0.5 | 0.5 |
| 2032 | 0.5 | 0.5 | 0.5 | 0.5 | 0 | 0 |
| 2033 | 0 | 0 | 0.5 | 0.5 | 0 | 0 |
| 2034 | 0 | 0 | 0.5 | 0.5 | 0.5 | 0 |
| 2035 | 0 | 0 | 0.5 | 0 | 0.5 | 0.5 |
| 2036 | 0 | 0 | 1 | 1 | 2 | 1 |
| 2037 | 0.5 | 0.5 | 1 | 1³ | 2 | 2 |

¹Four-tenths of a mL of the test substance was applied as received.
²Hours after induction dose.
³Desquamation of the dose site noted.

TABLE 74
Induction Phase Skin Reaction Scores (Positive Control - DNCB,
Historical Control)

| Induction Number | | |
|---|---|---|
| 1 | 2 | 3 |
| Concentration¹ | | |
| 0.08% | 0.08% | 0.08% |
| Amount Applied (mL) | | |
| 0.4 | 0.4 | 0.4 |

Hours²

| | 24 | 48 | 24 | 48 | 24 | 48 |
|---|---|---|---|---|---|---|
| Animal No. | | | | | | |
| 781 | 0.5 | 1 | 2 | 2 | 2 | 2 |
| 782 | 1 | 1 | 2 | 2 | 2 | 2 |
| 783 | 0.5 | 0.5 | 2 | 2 | 3 | 3 |
| 784 | 0.5 | 1 | 2 | 1 | 2 | 2 |
| 785 | 0.5 | 0.5 | 2 | 2 | 2 | 2 |
| 786 | 0.5 | 0.5 | 2 | 2 | 2 | 3 |
| 787 | 0.5 | 0.5 | 2 | 2 | 3 | 3 |
| 788 | 1 | 1 | 2 | 1 | 3 | 3 |
| 789 | 0 | 0 | 2 | 2 | 2 | 2 |
| 790 | 0.5 | 0.5 | 2 | 2 | 2 | 2 |

¹The positive control (DNCB) was applied as a 0.08% w/w solution in 80% aqueous ethanol.
²Hours after induction dose.

TABLE 75
Challenge Phase Skin Reaction Scores (Test Substance¹, Test
Animals)

| | |
|---|---|
| Animal No. | Hours² |

| Test | 24 | 48 |
|---|---|---|
| 2018 | 0.5 | 0.5 |
| 2019 | 0 | 0 |
| 2020 | 0.5 | 0 |
| 2021 | 0.5 | 0.5 |
| 2022 | 0.5 | 0 |
| 2023 | 0 | 0 |
| 2024 | 0 | 0 |
| 2025 | 0 | 0 |
| 2026 | 0.5 | 0.5 |
| 2027 | 0 | 0 |
| 2028 | 0.5 | 0.5 |
| 2029 | 0.5 | 0.5 |
| 2030 | 0.5 | 0.5 |
| 2031 | 0 | 0 |
| 2032 | 0 | 0 |
| 2033 | 0 | 0 |
| 2034 | 0.5 | 0 |
| 2035 | 0 | 0 |
| 2036 | 0.5 | 0.5 |
| 2037 | 0 | 0 |

¹Four-tenths of a mL of the test substance was applied as received.
²Hours after challenge dose.

TABLE 76
Challenge Phase Skin Reaction Scores (Test Substance¹, Naïve
Control)

| | |
|---|---|
| Animal No. | |
| Naïve | Hours² |

| Control | 24 | 48 |
|---|---|---|
| 2038 | 0 | 0 |
| 2039 | 0.5 | 0.5 |
| 2040 | 0 | 0 |
| 2041 | 0 | 0 |
| 2042 | 0.5 | 0 |
| 2043 | 0 | 0 |
| 2044 | 0.5 | 0.5 |
| 2045 | 0.8 | 0 |
| 2046 | 0 | 0 |
| 2047 | 0.5 | 0 |

¹Four-tenths of a mL of the test substance was applied as received.
²Hours after challenge dose.

TABLE 77
Challenge Phase Skin Reaction Scores (Positive Control -
DNCB¹, Historical Data)

Hours²

| Animal No. | 24 | 48 |
|---|---|---|
| Test | | |
| 781 | 0.5 | 0.5 |
| 782 | 1 | 1 |
| 783 | 2 | 2 |
| 784 | 1 | 1 |
| 785 | 1 | 0.5 |
| 786 | 2 | 2 |
| 787 | 1 | 2 |
| 788 | 2 | 2 |
| 789 | 0.5 | 0.5 |
| 790 | 2 | 2 |
| Naive Control | | |
| 791 | 0 | 0 |
| 792 | 0 | 0 |
| 793 | 0.5 | 0 |
| 794 | 0 | 0 |
| 795 | 0.5 | 0 |

¹Four-tenths of a mL of DNCB was applied as a 0.04% w/w solution in acetone.
²Hours after challenge dose.

### Example 21

### Therapeutic Effect of ASC-20N Patients Suffering from Various Dermal Conditions

This example demonstrates the effectiveness of antiseptic compositions according to the invention to treat dermal conditions. The effectiveness of the antiseptic compositions of the invention for the treatment of dermal conditions was tested as described in the following case studies. These studies were carried out with physician supervision. ASC-2 was prepared as described in Example 2. Patients suffering from different types of dermal conditions, as described below, were instructed by a physician to spread ASC-2 sparingly onto the area affected by the dermal condition twice a day for 1 week. Details of each case study with respect to the type of dermal condition and preliminary results of treatment are described below.

### Case Study A:

Patient #1 had severe eczema on his legs bilaterally for years. Patient #11 was treated by many doctors with strong topical steroids such as Betamethasone with little success. A nonsteroidal cream, Elidel, a topical immunomodulator, has also been tried with no success. The patient's skin had thickened and was oozing yellow serous fluid, most likely due to a Staphylococcus infection. Bactroban cream, a wide spectrum topical antibiotic, was also used with no benefit. ASC-2 was then tried and an immediate benefit was demonstrated when Patient #1 appeared for his next visit 4 days later. There was no active infection, the eczema was improving and the thickened skin was softening and thinning. In a final follow-up 3 months later, the patient did not need any creams on his legs and his skin was healing well. No thick eczematous skin remains.

### Case Study B:

Patient #2 had a Tinea versicolor rash on her trunk. A trial of ASC-2 provided no effect after 1 week. Patient #2 then saw a dermatologist and was given Nizoral cream for 3 weeks. Given the success observed in the other case studies, it is predicted that with an increase in the length of treatment or use of an antiseptic composition with a higher concentration of cortisone, or both, the patient would have derived some therapeutic benefit from the treatment. Alternatively, an antiseptic composition prepared with a different steroid, for example, betamethasone (such as for example, ASC-3 or ASC-4), may have proved effective. It is well known that the same infection in different people may react differently to any treatment, therefore, in cases like this, further studies are required to determine the best treatment regime.

### Case Study C:

Patient #3 had a Tinea Versicolor rash on her chest wall. A trial with Ketaderm cream provided no effect after 1 week. A trial with ASC-2 resolved the rash within 1 week.

### Case Study D:

Patient #4 had severe Athlete's Foot. A trial with ASC-2 provided significant benefit after 1 week, at which time the treatment was changed to Lotriderm (Clotrimazole+Betamethasone). No follow up on this patient is available. Although resolution of the condition was not obtained during the 1 week trial period, it is predicted that this patient may have benefited from a combination of the treatment with an antiseptic composition comprising betamethasone (such as, for example, ASC-3 and ASC-4) and a longer treatment time. Again, as indicated above, different patients respond differently to the same treatment and further research is required with different treatment times and antiseptic compositions to determine a treatment regime that would provide an even better outcome.

### Case Study E:

Patient #5 had a case of eczema with a secondary Staphylococcus infection. Treatment with ASC-2 caused significant improvement in the patient's condition in 1 week. Although resolution of the condition was not obtained during the 1 week trial period, it is predicted that treatment for a longer period of could provide an even better outcome, as shown in Case study A, and for the reasons indicated in Case studies B and D.

### Case Study F:

Patient #6 had Athlete's Foot. Treatment with ASC-2 resolved the condition in 1 day.

The preceding case studies indicate that treatment with ASC-2 was able to provide substantial benefit, and in many cases complete resolution, of dermal conditions including eczema accompanied by bacterial infection, rashes caused by Tinea Versicolor infection, and athlete's foot.

### Example 22

### Therapeutic Effect of Asc-10R Asc-20N Dermal or Cuticle Conditions

This example demonstrates the effectiveness of antiseptic compositions of the invention in treating dermal conditions caused by fungi, bacteria, or viruses. The effectiveness of the antiseptic compositions of the invention for the treatment of dermal conditions was tested on several individuals as described in the following case studies. ASC-1 was prepared as described in Example 1, while ASC-2 was prepared as described in Example 2. Details of each case study with respect to the type of dermal condition and preliminary results of treatment are described below.

Case study #1—The individual was an adult male with occasionally recurring itchy, red infection, assumed to be Athlete's Foot, between all toes on both feet. When the infection occurred, the individual applied ASC-1 between the toes which normally eliminated the itching and redness with one application or, at most, 2 applications within 12 hours. In a separate, more severe incident, painful cracking of the skin between the toes of both feet occurred. ASC-2 was applied to the left foot and ASC-1 was applied to the right foot. ASC-2 cleared up the infection in 2 days and ASC-1 cleared up the infection in 3 days.

Case study #2—The individual was an adult male with occasionally recurring itchy, red infection, assumed to be Athlete's Foot, between all toes on both feet. When the infection occurred, the individual applied ASC-1 between toes after showering which normally eliminated the itching and redness with one application or, at most, 2 applications within 12 hours.

Case Study #3—The individual was an adult male suffering from chronic Athlete's Foot between all toes on both feet and partially under the toenails for over 30 years. The individual applied ASC-1 twice between toes and under the toenails within a 12 hour period. After a 5 day follow up, the infection had not returned.

Case study #4—The individual was an adult female with an infection that had proceeded halfway under the toenail on one large toe, causing discoloration and pain. ASC-1 was applied on the toe and under the toenail once a day, after bathing, for a period of two weeks. Within the two weeks, the pain was gone. After two months, the toenail had grown out, the discoloration was gone and toenail appeared to be normal.

Case study #5—The individual was an adult male with an infection from a cut beside the fingernail on the index finger. The infection was quite sore, and was spreading and moving under the fingernail. The individual applied ASC-1 three times over a 12 hour period on Day 1 which eliminated the pain. Three more applications of ASC-1 were performed over a 12 hour period on Day 2 which eliminated the infection by Day 3.

Case study #6—The individual was an adult male with erupted surface acne on his face. ASC-2 was applied to the blemishes twice within 12 hours on Day 1 and a significant reduction in the inflammation occurred overnight. Two more applications over a 12 hour period on Day 2 eliminated the blemishes by Day 3. However, when applied to a blemish that had not yet erupted, the formulation had no effect. As the formulation was successful in eliminating blemishes that had erupted, it is predicted that a change in the mode of application, such as for example, use of a trans-dermal delivery system to penetrate the skin, could be used to successfully treat infections below the surface of the skin.

Case study #7—The individual was an adult female with cold sores, most likely caused by Herpes simplex 1. ASC-1 was applied approximately 5 times per day directly onto the cold sores. Within 3 days, the cold sores had disappeared.

Case study #8—The individual was an adult female who developed an infection under three fingernails. Over the counter products were not successful in eliminating the infection and the individual's doctor was not able to suggest alternate treatments. The individual applied ASC-1 with a Q-Tip directly to the fingernails which eliminated the infection. The individual's report did not include how many times per day or for how many days the infection was treated but, given the relatively short treatment periods required to eliminate infection in the other case studies, it is likely that in this case study the treatment was not carried out for an extended period of time.

Case study #9—The individual was an adult male with a severe, unidentified painful infection of both feet, assumed to be Athlete's Foot, that caused the skin to crack and peel. ASC-1 was applied to the skin of both feet an unspecified number of times per day and a few days later, the soreness and cracking of the skin had disappeared. The condition has returned on occasion but is kept in check with immediate applications of ASC-1.

Case study #10—The individual was an adult female with an infection that caused swelling on the index finger of the right hand. The infection was diagnosed as cellulitis, a bacterial infection. She was given two separate courses of broad-spectrum antibiotics by her physician with no effect. On a third visit to her physician, the infection was incised and drained. On inspection, the physician decided that the infection was fungal in origin. To treat the infection, her finger was soaked in small jar filled with ASC-1 three to four times daily, for 15 minutes each time. After 4 weeks, the infection had completely disappeared, with no recurrence.

### Example 23

### Evaluation of Fungicidal Activity of an Antiseptic Composition Against Trichophyton Mentagrophytes and Trichophyton Rubrum

SUMMARY: The following is an exemplary method by which the fungicidal activity of an antiseptic composition according to the invention can be tested. The following In-Vitro Time-Kill Method is used to assess the fungicidal activity of an antiseptic composition according to the invention when challenged with suspensions of two mycelial fungal species, and is based on a modification of the methods described in the Draft European Standard, prEN 13624, “Chemical Disinfectants and Antiseptics—Quantitative Suspension Test for the Evaluation of Fungicidal Activity of Chemical Disinfectants for Instruments Used in the Medical Area—Test Method and Requirements (2003).” Exemplary fungal strains that can be used in this study are American Type Culture Collection (ATCC) Trichophyton mentagrophytes (ATCC #95333) and Trichophyton rubrum (ATCC #28188).

As indicated above, the following method is exemplary, and as such, any measurements, including those of volume, time, temperature, and cell numbers, and any identification of specific growth media noted in the following protocol are included as suitable examples of same and may be adjusted as necessary by the skilled worker in light of the specific circumstances under which the experiments are conducted.

### Methodology:

Test product: An antiseptic composition according to the invention.

Inoculum Preparation: Approximately 7 to 14 days prior to testing, inocula from lyophilized vials containing T. mentagrophytes and T. rubrum is suspended in Tryptone Sodium Chloride Solution (TSC), inoculated onto the surface of Emmon's Sabouraud Dextrose Agar (ESDA) contained in Petri plates and incubated appropriately until sufficient growth was observed. This produces lawns of the challenge fungi on the surface of the agar plates and conidia from these are used to prepare the Test and Validation Suspensions.

Fungal Test (N) and Validation (Nᵥ) Suspensions: Prior to initiating the Fungicidal Assay Procedure and the Method Validation Procedure, suspensions of T. mentagrophytes and T. rubrum are prepared in a solution of sterile Water-for-Irrigation, USP, with 0.05% (v/v) Polysorbate 80 (WFI-P80). Polysorbate is used to aid in dispersal of the conidia. The conidia are detached from the surface of the solid media by gently scraping with a sterile spatula or spreader, suspended in approximately 10 mL of WFI-P80 and transferred to a sterile flask containing glass beads. The flasks are gently shaken by hand for about 1 minute and then the contents filtered through sterile cheesecloth, as necessary, to separate the gross debris and/or mycelial fragments. The suspensions are visually examined under magnification to confirm that no mycelial fragments or germinated spores were present (at least approximately 10 fields of view are checked). If germinated spores are present, the suspensions are discarded. If mycelial fragments are present, the suspensions are “washed” a minimum of about 2 times by centrifuging, the supernatant is removed and the pellets are resuspended in TSC. The fungal spore suspensions are diluted, as necessary, with additional TSC to produce concentrations of approximately 1.5×10⁷ to 5×10⁷ CFU/mL for use in the Fungicidal Assay Procedures. Suspensions containing approximately 4.5×10² to 1.8×10³ CFU/mL are prepared for the Method Validation Procedure. The suspensions are stored at 2° C. to 8° C. for up to about 2 days prior to use, as required.

FUNGICIDAL ASSAY PROCEDURE: Prior to testing, the test product (ASC-1), the Test Suspensions, the interfering substance and all diluting fluids are placed in a water bath at approximately 20° C.±1° C. and allowed to equilibrate for no less than 15 minutes or until the temperature of the solutions stabilizes at about 20° C.±1° C.

Initial Population Determination—Fungal Test Suspensions (N): A 1.0 mL (approximately) aliquot of a Test Suspension containing approximately 1.5×10⁷ to 5×10⁷ CFU/mL is transferred into a sterile test tube containing about 9.0 mL of TSC and mixed thoroughly (10⁻¹ dilution). Ten-fold dilutions (e.g. 10⁻², 10⁻³, 10⁻⁴, 10⁻⁵ and 10⁻⁶) of each suspension are prepared in TSC. Using Emmon's Sabouraud Dextrose Agar with product neutralizers, (ESDA+, product neutralizers as described in Example 5) spread plates are then prepared by plating duplicate aliquots (for example, about 1.0 mL) of the final dilutions (e.g. 10⁻⁵ and 10⁻⁶) of each Test Suspension. The plates are incubated at about 25° C.±2° C. for T. mentagrophytes and about 30° C.±2° C. for T. rubrum for approximately 2 to 10 days or until sufficient growth is observed.

Testing Procedure—Fungicidal Test Mixt N: A1.0 mL (approximately) aliquot of a Test Suspension containing approximately 1.5×10⁷ to 5×10⁷ CFU/mL is transferred into a sterile test tube containing about 1.0 mL of Bovine Albumin Solution (BAS), mixed thoroughly and replaced in the water bath at about 20° C.±1° C. The tube remains in the water bath for about 2 minutes. Then an approximately 8.0 mL aliquot of the test product, ASC-1, is added to the tube containing BAS/Test Suspension and the tube is replaced in the water bath. Each of the two fungal species is exposed to the test product/BAS mixture at about 20° C.±1° C. for a suitable period of time, such as 5 minutes, 15 minutes, 30 minutes and 60 minutes, for example, and timed using a calibrated minute/second timer. After each exposure time elapses, about 1.0 mL is removed from each tube containing test product/BAS/Test Suspension, placed in separate sterile test tubes containing approximately 8.0 mL of Neutralizing Formulation (NF) and approximately 1.0 mL of Water-for-Irrigation, USP (WFI) and mixed thoroughly with a vortex mixer. The tubes are allowed to remain in the water bath for an appropriate neutralization time of, for example, 5 minutes, timed with a calibrated minute/second timer. Following the neutralization time, suitable aliquots (for example 1.0 mL), in duplicate, of the neutralized test mixture are spread-plated using ESDA+. The plates are incubated at about 25° C.±2° C. for T. mentagrophytes and about 30° C.±2° C. for T. rubrum for approximately 2 to 10 days, or until sufficient growth is observed.

Data Collection (V_{c}-values): Following incubation, the colonies on the plates are counted manually using a hand-tally counter. Counts in the range of 14 to 165 colony-forming units (CFU) are used preferentially in the calculation of the fungal populations.

METHOD VALIDATION: DILUTION-NEUTRALIZATION PROCEDURE: Fungal Validation Suspensions containing approximately 4.5×10² to 1.8×10³ CFU/mL of each challenge species are prepared by diluting the fungal spore suspensions, as necessary, with additional TSC. Prior to testing, the test product, Validation Suspensions, interfering substance (Bovine Albumin Solution—BAS) and diluting fluids (Neutralizing Formulation of Butterfield's Phosphate Buffer with product neutralizers—NF; Tryptone Sodium Chloride Solution TCS, and Sterile Water-for-Irrigation—WFI) are placed in a water bath at 20° C.±1° C. and allowed to equilibrate for no less than about 15 minutes, or until the temperature of the solutions stabilizes to about 20° C.±1° C.

Initial Population Determination—Fungal Validation Suspensions (N_{y})—Test “C”: A suitable aliquot (approximately 1.0 mL, for example) of a Validation Suspension containing approximately 4.5×10² to 1.8×10³ CFU/mL is transferred into a sterile tube containing approximately 9.0 mL of TSC and mixed thoroughly. A suitable aliquot (about 1.0 mL, for example), in duplicate, of this suspension is spread-plated using ESDA+. NOTE: Each aliquot of suspension is divided into portions of approximately equal size and distributed into 2 plates. The plates are incubated at about 25° C.±2° C. for T. mentagrophytes and about 30° C.±2° C. for T. rubrum for approximately 2 to 10 days, or until sufficient growth is observed.

Experimental Conditions Control (A)—Test “E”: A suitable aliquot, for example 1.0 mL, of a Validation Suspension containing approximately 4.5×10² to 1.8×10³ CFU/mL is transferred into a sterile tube containing about 1.0 mL of BAS, mixed thoroughly and replaced in the water bath at approximately 20° C.±1° C. for about 2 minutes. Then an 8.0 mL aliquot of sterile WFI is added to the tube containing BAS/Validation Suspension and the tube is replaced in the water bath. Each challenge fungal strain is exposed to the WFI/BAS mixture at 20° C.±1° C. for about 60 minutes, timed using a calibrated minute/second timer. Following this exposure, duplicate aliquots, for example 1.0 mL, of this suspension are spread-plated using ESDA+. The plates are incubated at approximately 25° C.±2° C. for T. mentagrophytes and approximately 30° C.±2° C. for T. rubrum for about 2 to 10 days, or until sufficient growth is observed.

Neutralizer Control—Validation of the Non-Toxicity of the Neutralizing Medium (B)—Test “B”: A suitable aliquot, for example about 1.0 mL, of a Validation Suspension containing approximately 4.5×10² to 1.8×10³ CFU/mL is transferred into a sterile tube containing approximately 8.0 mL of NF and approximately 1.0 mL of sterile WFI, mixed thoroughly and replaced in the water bath at about 20° C.±1° C. for a suitable period of time, such as approximately 60 minutes. Each challenge species is exposed to the NF/WFI mixture at about 20° C.±1° C. for about 5 minutes. Then duplicate aliquots (for example about 1.0 mL) of this suspension are spread-plated using. ESDA+. The plates are incubated at about 25° C.±2° C. for T. mentagrophytes and about 30° C.±2° C. for T. rubrum for approximately 2 to 10 days, or until sufficient growth is observed.

Method Validation—Dilution Neutralization (C)—Test “A”: A suitable aliquot, for example 1.0 mL, of TSC is transferred into a sterile tube containing approximately 1.0 mL of BAS and mixed thoroughly. An approximately 8.0 mL aliquot of the test product is added to the TSC/BAS mixture, vortexed thoroughly and replaced in the water bath at 20° C.±1° C. for a suitable period of time, for example, 60 minutes. Then an approximately 1.0 mL aliquot is removed from the tube containing diluent/BAS/test product and is transferred into a sterile tube containing approximately 8.0 mL of NF. The tube is mixed thoroughly and replaced in the water bath for about 5 minutes. Then approximately 1.0 mL of a Validation Suspension containing approximately 4.5×10² to 1.8×10³ CFU/mL is transferred into the tube containing diluent/BAS/test product/NF and mixed thoroughly. The tube is replaced in the water bath at about 20° C.±1° C. for about 30 minutes. Following this exposure time, duplicate aliquots of, for example, about 1.0 mL, from the tube are spread-plated using ESDA+. The plates are then incubated at approximately 25° C.±2° C. for T. mentagrophytes and approximately 30° C.±2° C. for T. rubrum for about 2 to 10 days, or until sufficient growth is observed.

A summary of the preceding testing procedures is shown in Table 78

Data Collection (V_{c}-values): Following incubation, the colonies on the plates are counted manually using a hand-tally counter. Counts in the range of 14 to 165 colony-forming units (CFU) are used preferentially in the calculations of fungal populations. NOTE: All data are reported as V_{c}-values (i.e. number of CFU counted per 1.0 mL sample).

CALCULATIONS/REPORTING OF RESULTS: Table 79 provides a summary of the terms used in the calculation of results.

TABLE 78
Summary of Terms Used In Calculations

| | | | Number of cells |
|---|---|---|---|
| | | | per mL in |
| | | Number of cells | the test mixture |
| | | per mL in | at the end of |
| | Number of cells | the test mixture | contact time |
| | per mL in | at the beginning | (60, min for A, |
| | the fungal | of contact time | 5 min for B, |
| | suspensions | (Time = 0) | 30 min for C) |
| Test | N - Test | Nₒ | |
| | Suspension | (Nₒ = N/10) | |
| Controls | Nᵥ - Validation | Nᵥₒ | Nₐ |
| | Suspension | (Nᵥₒ = Nᵥ/10) | A, B, C |

All plate counts are reported as V_{c}-values. N and Nᵥ represent the fungal Test and Validation Suspensions, respectively. Nₐ represents the Fungicidal Test Mixture. A (Experimental Conditions Control), B (Neutralized Control) and C (Method Validation Control) represent the different control test mixtures. N, Nᵥ, Nₒ, Nᵥₒ, Nₐ and A, B, C represent the number of CFU counted per mL in the different test mixtures.

Calculation of N and Nₐ: Because 2 dilutions of the Test Suspension are evaluated, the number of CFU/ml is calculated as the weighted average count, as follows:

$$N = \frac{c}{( {n}_{1} + 0.1 {n}_{2} )} {10}^{- 5}$$

Where c=sum of the V_{c} values taken into account
n₁=number of V_{c}-values taken into account in the lower dilutions (i.e. 10⁻⁵)
n₂=number of V_{c}-values taken into account in the higher dilutions (i.e. 10⁻⁵)
10⁻⁵=dilution factor corresponding to the lower dilution.

Nₒ is the number of CFU/mL in the test mixture at the beginning of the contact time. It is one-tenth of the weighted mean of N due to the 10-fold dilution introduced by the addition of the product and interfering substance. The target range for N is about 1.5×10⁷ to 5.0×10⁷ of each challenge species. The target range for Nₒ is approximately 1.5×10⁶ to 5.0 to 10⁶ CFU/mL of each challenge species.

Calculation of Nₐ: Nₐ is the number of survivors per mL in the test mixture at the end of the contact time and prior to neutralization. It is 10-fold higher than the V, values due to the addition of neutralizer and water, and is calculated as follows:

$${N}_{a} = \frac{c \times 10}{n}$$

where c=sum of the V_{c} values taken into account
n=number of V_{c}-values taken into account

Calculation of Nᵥ and Nᵥₒ: Nᵥ is the number of CFU/mL in the Validation Suspension. It is 10-fold higher than the counts in terms of V_{c}-values due to the 10⁻¹ dilution step. N^{vo} is the number of CFU-mL in the mixtures A, B, C at Time=0. It is one-tenth of the mean of the V_{c}-values of Nᵥ.

$${N}_{v} = \frac{c \times 10}{n}$$

$${N}_{vo} = \frac{c}{n}$$

where c=sum of the V_{c} values taken into account
n=number of V_{c}-values taken into account

The target range for Nᵥₒ is 4.5×10¹ to 1.8×10² CFU/mL of each challenge species.

The target range for Nᵥ is 4.5×10² to 1.8×10³ CFU/mL of each challenge species.

Calculation of A, B, C: A, B, C are the numbers of survivors in the Experimental Conditions Control, the Neutralizer Control and Method Evaluation Control at the end of the appropriate contact times.

$$A , B , C = \frac{c}{n}$$

where c=sum of the V_{c} values taken into account
n=number of V_{c}-values taken into account
   A, B and C must be equal or greater than 0.5×Nᵥₒ.

Calculation of Log₁₀ Reductions: The Log₁₀ reduction (R) of each fungal challenge species is calculated as follows:

R=Log₁₀(Nₒ/Nₐ)

TABLE 79
Summary Of Testing Methodology

| Test A | Test B | | | Test E |
|---|---|---|---|---|
| Neutralization | Neutralization | Test C | | Neutralization |
| Validation & | Validation & | Neutralization | Test D | Validation & |
| Neutralizer | Neutralizer | Validation & Test | Fungicidal | Experimental |
| Effectiveness | Toxicity | Organism Viability | Efficacy | Conditions Control |
| Interfering | NF | TSC | Interfering | Interfering |
| Substance | | | Substance | Substance |
| TSC | Test Organism | Test Organism | Test | Test |
| | | | Organism | Organism |
| ASC-1 | Hold/Expose | Plate Count | Hold/Expose | Hold/Expose |
| Hold/Expose | Plate Count | | ASC-1 | WFI |
| NF | | | Hold/Expose | Hold/Expose |
| Hold/Expose | | | NF | Plate Count |
| Test Organism | | | Hold/Expose | |
| Hold/Expose | | | Plate Count | |
| Plate Count | | | | |

### Example 24

### Evaluation of Antimycobacterial Activity of An Antiseptic Composition Against Mycobacterium Avium and Mycobacterium Terrae

SUMMARY: The following is an exemplary method by which the mycobactericidal activity of an antiseptic composition according to the invention can be tested. An In-Vitro Time-Kill Method is used to assess the mycobactericidal activity of an antiseptic composition when challenged with suspensions of two different mycobacterial strains. An In-vitro Time-Kill evaluation is performed using a modification of the methods described in the European Standard, EN 14348 “Chemical Disinfectants and Antiseptics—Quantitative Suspension Test for the Evaluation of Mycobatericidal Activity of Chemical Disinfectants in the Medical Area Including Instrument Disinfectants—Test Method and Requirements (2005).”

As indicated above, the following method is exemplary, and as such, any measurements, including those of volume, time, temperature, and cell numbers, and any identification of specific growth media noted in the following protocol are included as suitable examples of same and may be adjusted as necessary by the skilled worker in light of the specific circumstances under which the experiments are conducted.

### Methodology:

Test Substance: An antiseptic composition according to the invention.

Mycobacterial strains: Mycobacterium avium (ATCC #15769) and M. terrae (ATCC #15755)

Inoculum Preparation: Prior to testing, inocula from lyophilized vials containing the challenge species are suspended in an appropriate broth such as Middlebrook 7H₉Broth with 10% ADC Enrichment (MADC), inoculated onto the surface of Middlebrook and Cohn 7H10 Agar with 10% OADC Enrichment (MCO) in Petri plates, and incubated at 35°±2° C. for approximately 21 days. Following incubation, the organisms are recovered from the agar plates by suspending the growth in MADC. These suspensions are dispensed into cryovials and maintained at −70° C. for prolonged storage, as necessary. Then at a suitable time, at for example, 21 days prior to testing, inocula from the cryovials containing each species are inoculated on the surface of MCO in the Petri plates. These plates are incubated at 35°±2° C. for approximately 21 days, or until sufficient growth is observed and they produce lawns of the challenge microorganisms on the surface of the agar plates. Microbial growth from these is used to prepare the Test and Validation Suspensions.

Test (N) and Validation (N_{V}) Suspensions: Prior to initiating the Mycobactericidal Assay Procedure and the Method Validation Procedure, a suspension of each challenge species is prepared in sterile Water-for-Irrigation, USP (WFI). A sterile inoculating loop is used to transfer the growth from the solid media into sterile centrifuge tubes containing dry glass beads. The cultures are homogenized by mixing for at least 5 minutes to distribute the cells evenly on the beads and on the internal surface of the tube. WFI is gently added to the tubes containing each culture and mixed thoroughly. After about a 20 minute sedimentation period, the supernatant is removed and transferred to a sterile container. These bacterial suspensions are diluted, as necessary, with additional WFI to produce concentrations of approximately 1.5×10⁹ to 5×10⁹ CFU/ml, for use in the Mycobactericidal Assay Procedure—Test Suspensions (A). Suspensions containing approximately 3×10² to 1.6×10³ CFU/mL are prepared for the Method Validation Procedure—Validation Suspensions (Nᵥ). These suspensions are used on the day of their preparation.

MYCOBACTERICIDAL ASSAY PROCEDURE: Prior to testing, the test product, the Test Suspensions, the interfering substance (Bovine Albumin Solution—BAS), and all diluting fluids are placed in a water bath at 20°±1° C. and allowed to equilibrate for about 15 minutes, or until the temperature of the solutions stabilizes at 20°±1° C.

Initial Population Determination: Test Suspensions—(N): A aliquot (for example 1.0 mL) of a Test Suspension containing approximately 1.5×10⁹ to 5×10⁹ CFU/mL is transferred into a sterile test tube containing about 9.0 mL of WFI and mixed thoroughly. Ten-fold dilutions (e.g., 10⁻², 10⁻³, 10⁻⁴, 10⁻⁵, 10⁻⁶, 10⁻⁷, and 10⁻⁸) of each suspension are prepared in WFI. Using MCO, spread-plates are prepared by plating duplicate aliquots of the final dilutions, for example about 1.0 mL, (e.g., 10⁷ and 10⁻⁸) of each Test Suspension. The plates are incubated at 35°±2° C. for 21 days, or until sufficient growth is observed.

Testing Procedure—Mycobactericidal Test Mixture—(Nₐ)—Test “D”: A aliquot (for example 1.0 mL) of a Test Suspension containing approximately 1.5×10⁹ to 5×10⁹ CFU/mL is transferred into a sterile test tube containing about 1.0 mL of Bovine Albumin Solution (BAS), mixed thoroughly, and placed in the water bath at 20°±1° C. for 2 minutes. Then an approximately 8.0 mL aliquot of test product is added to the tube containing BAS/Test Suspension and the tube replaced in the water bath. Each challenge strain is exposed to the test product/BAS mixture at 20°±1° C. for periods of time such as 5 minutes, 15 minutes, 30 minutes and 60 minutes, timed using a calibrated minute/second timer. After each exposure time has elapsed, a suitable aliquot (for example, 1.0 mL) is removed from each tube containing product/BAS/Test Suspension, placed in separate sterile test tubes containing 8.0 mL of Neutralizing Formulation (NF, for example, as described in Example 5) and 1.0 mL of WFI, and mixed thoroughly using a vortex mixer. The tube is allowed to remain in the water bath for a neutralization time of approximately 5 minutes, timed with a calibrated minute/second timer. Then duplicate aliquots (for example, 1.0 mL) of the neutralized test mixture are spread-plated using MCO. Additionally, a approximately 0.5 mL aliquot of each test mixture Nₐ is serially diluted in approximately 4.5 mL of NF to produce 10⁻¹, 10⁻², and 10⁻³ dilutions of Nₐ. Duplicate 1.0 mL aliquots of each dilution are spread-plated using MCO, as described above. The plates are incubated at 35°±2° C. for 21 days, or until sufficient growth is observed.

Data Collection—V_{c}-values: Following incubation, the colonies on the plates are counted manually using a hand-tally counter. Counts in the range of 14 to 330 CFU are used preferentially in the data calculations. Note: All data are reported as V_{C}-values; a V_{C}-value is the number of CFU counted per 1.0 mL sample.

METHOD VALIDATION: DILUTION-NEUTRALIZATION PROCEDURE: Validation Suspensions containing approximately 3×10² to 1.6×10³ CFU/mL of a challenge species are prepared as described in Test (N) and Validation (N_{V}) Suspensions. Prior to testing, the test product, the Validation Suspensions, the interfering substance, and all diluting fluids are placed in a water bath at 20°±1° C. and allowed to equilibrate for no less than 15 minutes, or until the temperature of the solutions stabilizes at 20°±1° C.

Initial Population Determination—Validation Suspensions—(Nᵥ)—Test “C”: A suitable aliquot, such as 1.0 mL, of a Validation Suspension containing approximately 3×10² to 1.6×10³ CFU/mL is transferred into a sterile tube containing 9.0 mL of WFI and mixed thoroughly. Duplicate 1.0 mL aliquots of this suspension are spread-plated using MCO. The plates are incubated at 35°±2° C. for 21 days, or until sufficient growth is observed.

Experimental Conditions Control—(A)—Test “E”: A 1.0 mL aliquot of a Validation Suspension containing approximately 3×10² to 1.6×10³ CFU/mL is transferred into a sterile tube containing 1.0 mL of BAS, mixed thoroughly, and replaced in the water bath at 20°±1° C. The tube remains in the water bath for 2 minutes, then an 8.0 mL aliquot of sterile WFI is added to the tube and it is replaced in the water bath. Each challenge strain is exposed to the WFI/BAS mixture at 20°±1° C. for 60 minutes, timed using a calibrated minute/second timer, and then duplicate 1.0 mL aliquots of the suspension are spread-plated using MCO. The plates are incubated at 35°±2° C. for 21 days, or until sufficient growth is observed.

Neutralizer Control (Validation of the Non-Toxicity of the Neutralizing Medium)—(B)—Test “B”: A 1.0 mL aliquot of a Validation Suspension containing approximately 3×10² to 1.6×10³ CFU/mL is transferred into a sterile tube containing 8.0 mL of NF and 1.0 mL of sterile WFI, mixed thoroughly, and replaced in the water bath at 20°±1° C. Each challenge species is exposed to the NF/WFI mixture at 20°±1° C. for 5 minutes and then duplicate 1.0 mL aliquots of this suspension are spread-plated using MCO. The plates are incubated at 35°±2° C. for 21 days, or until sufficient growth is observed.

Method Validation—Dilution Neutralization—(C)— Test “A”: A 1.0 mL aliquot of WFI is transferred into a sterile test tube containing 1.0 mL of BAS and mixed thoroughly. An 8.0 mL aliquot of the test product is added to the WFI/BAS mixture, vortexed thoroughly and replaced in the water bath at 20°±1° C. for 60 minutes. Then a 1.0 mL aliquot is removed from the tube containing diluent/BAS/product and transferred into a sterile tube containing 8.0 mL of NF. The tube is mixed thoroughly and replaced in the water bath for 5 minutes. Then a 1.0 mL aliquot of a Validation Suspension containing approximately 3×10² to 1.6×10³ CFU/mL is transferred into the tube containing diluent/BAS/product/NF and mixed thoroughly. The tube is replaced in the water bath at 20°±1° C. for 30 minutes. Then duplicate 1.0 mL aliquots from this tube are spread-plated using MCO. The plates are incubated at 35°±2° C. for 21 days, or until sufficient growth is observed.

Data Collection: V_{C}-values: Following incubation, the colonies on the plates are counted manually using a hand-tally counter. Counts in the range of 14 to 330 CFU are used preferentially in the data calculations.

### Calculations/Reporting of Results:

Table 80 provides a summary of the terms used in the calculation of results.

TABLE 80
Summary of terms

| | | | Number of cells |
|---|---|---|---|
| | | | per mL in the |
| | | Number of cells | test mixture |
| | | per mL in the | at the end of |
| | Number of cells | test mixture | contact time |
| | per mL in | at the beginning | (60 min for A, |
| | the fungal | of contact time | 5 min for B, |
| | suspensions | (Time = 0) | 30 min for C) |
| Test | N - Test | Nₒ | Nₐ |
| | Suspension | (Nₒ = N/10) | |
| Controls | Nᵥ - Validation | Nᵥₒ | A, B, C |
| | Suspension | (Nᵥₒ = Nᵥ/10) | |

All plate counts are reported as V_{c}-values, which is the number of CFU counted per 1.0 mL sample. N and Nᵥ represent the bacterial Test and Validation Suspensions, respectively. Nₐ represents the Mycobatericidal Test Mixture. A (Experimental Conditions Control), B (Neutralized Control) and C (Method Validation Control) represent the different control test mixtures. N, Nᵥ, Nₒ, Nᵥₒ, Nₐ and A, B, C represent the number of CFU counted per mL in the different test mixtures.

Calculation of N and Nₒ: Because 2 dilutions of the Test Suspension are evaluated, the number of CFU/ml is calculated as the weighted average count, as follows:

$$N = \frac{c}{( {n}_{1} + 0.1 {n}_{2} )} {10}^{- 7}$$

Where c=sum of the V_{c} values taken into account
n₁=number of V_{c}-values taken into account in the lower dilutions (i.e. 10⁻⁷)
n₂=number of V_{c}-values taken into account in the higher dilutions (i.e. 10⁻⁸)
10⁻⁷=dilution factor corresponding to the lower dilution.

Nₒ is the number of CFU/mL in the test mixture at the beginning of the contact time. It is one-tenth of the weighted mean of N due to the 10-fold dilution introduced by the addition of the product and interfering substance. The target range for N is 1.5×10⁹ to 5.0×10⁹ of each challenge species. The target range for Nₒ is 1.5×10⁸ to 5.0 to 10⁸ CFU/mL of each challenge species.

Calculation of Nₐ: Nₐ is the number of survivors per mL in the test mixture at the end of the contact time and prior to neutralization. It is 10-fold higher than the V_{c}-values due to the addition of neutralizer and water. Nₐ is calculated for each dilution step Nₐ⁰, N⁻¹, Nₐ⁻², Nₐ⁻³) as follows:

$${N}_{a} = \frac{c \times 10}{n}$$

where c=sum of the V_{c} values taken into account
n=number of V_{c}-values taken into account

Calculation of Nᵥ and Nᵥₒ: Nᵥ is the number of CFU/mL in the Validation Suspension. It is 10-fold higher than the counts in terms of V_{c}-values due to the 10⁻¹ dilution step. Nᵥₒ is the number of CFU-mL in the mixtures A, B, C at Time=0. It is one-tenth of the mean of the V_{c}-values of Nᵥ.

$${N}_{v} = \frac{c \times 10}{n}$$

$${N}_{vo} = \frac{c}{n}$$

where c=sum of the V_{c} values taken into account
n=number of V_{c}-values taken into account

The target range for Nᵥₒ is 3×10¹ to 1.6×10² CFU/mL of each challenge species. The target range for Nᵥ is 3×10² to 1.6×10³ CFU/mL of each challenge species.

Calculation of A, B, C: A, B, C are the numbers of survivors in the Experimental Conditions Control, the Neutralizer Control and Method Evaluation Control at the end of the appropriate contact times.

$$A , B , C = \frac{c}{n}$$

where c=sum of the V_{c} values taken into account
n=number of V_{c}-values taken into account
   A, B and C must be equal or greater than 0.5×Nᵥₒ. Calculation of Log₁₀Reductions: The Log₁₀ reduction (R) of each fungal challenge species is calculated as follows:

R=Log₁₀(Nₒ/Nₐ)

Table 81 provides a summary of the testing methodology that can be used in this example.

TABLE 81
Summary of the testing methodology

| | | | | Test E |
|---|---|---|---|---|
| | | | | Neutralization |
| Test A | Test B | Test C | | Validation & |
| Neutralization | Neutralization | Neutralization | Test D | Experimental |
| Validation & | Validation & | Validation & Test | Mycobatericidal | Conditions |
| Effectiveness | Toxicity | Organism Viability | Efficacy | Control |
| Interfering | NF | WFI | Interfering | Interfering |
| Substance | | | Substance | Substance |
| WFI | Test Organism | Test Organism | Test Organism | Test Organism |
| Antiseptic | Hold/Expose | Plate Count | Hold/Expose | Hold/Expose |
| composition | | | | |
| Hold/Expose | Plate Count | | Antiseptic | WFI |
| | | | composition | |
| NF | | | Hold/Expose | Hold/Expose |
| Hold/Expose | | | NF | Plate Count |
| Test Organism | | | Hold/Expose | |
| Hold/Expose | | | Plate Count | |
| Plate Count | | | | |

### Example 25

### Evaluation of Antiviral Properties of an Antiseptic Composition Against Human Papilloma Virus

SUMMARY: The following is an exemplary method by which the ability of the antiseptic compositions according to the invention to kill human papilloma virus can be tested. As this virus cannot be cultured or worked with in a laboratory setting, this example uses a surrogate virus, SV40 as the test virus. The virucidal activity of an antiseptic composition when challenged with the SV-40 virus is assessed using a Virucidal Suspension Test (In-Vitro Time-Kill). The Test Product is being evaluated at an 80% (v/v) concentration.

SCOPE: This study determines the antiviral efficacy of the Test Product when challenged with the surrogate for Human Papilloma Virus, Simian Virus 40 (SV-40; ATCC #1VR-281), using a modification of the European Standard, EN 14476 “Chemical Disinfectants and Antiseptics—Virucidal Quantitative Suspension Test for Chemical Disinfectants and Antiseptics Used in Human Medicine (2005).” The test is performed under clean conditions with Phosphate Buffered Saline (PBS) as an interfering substance. The Log₁₀ reductions from the initial population of the viral strain are determined following exposure to the test product for 30 seconds, 1 minute and 3 minutes. Plating is performed in 8 replicates.

Test Product: The test product is an antiseptic composition according to the invention.

HOST CELL PREPARATION: BS-C-1 (Cercopithecus aethiops), African green monkey kidney cells (ATCC #CCL-26) are maintained as monolayers in disposable cell culture labware in accordance with BSLI SOP L-2084, “Procedure for Subculturing Cells” and are used for the Virucidal Suspension Test. At 24 to 48 hours prior to testing, host cell cultures are seeded onto the appropriate culture plates. Cell monolayers need to be sufficiently confluent before inoculation with the virus. The Growth median (GM) and Maintenance medium (MM) are 1× Minimum Essential Medium (MEM) and/or 1× Dulbecco's Modified Eagle's Medium (DMEM) with appropriate supplements.

TEST VIRUS PREPARATION: Viruses from BSLI high-titer virus stock are used for this study. On the day of use, aliquots of the stock virus are removed from a −70° C. freezer and thawed for use in testing.

NEUTRALIZATION TEST: The Neutralization test includes the following parameters:

TABLE 82
Neutralization test parameters

| | | | | | Number of |
|---|---|---|---|---|---|
| | | | Interfering | Number | Replicates |
| | Antiseptic | Virus | Substances | of tests | per test |
| Neutralization Control | 80% (v/v) | ~100-1000 | PBS | 3 | 8 |
| (neutralized product vs. | | infectious | | | |
| virus) | | units | | | |
| Cytotoxicity Control | 80% (v/v) | none | PBS | 1 | 8 |
| (neutralized product vs. | | | | | |
| cell culture) | | | | | |
| Virus Control #1 | Lowest non- | Virus 10-fold | PBS | 1 | 8 |
| (neutralized product vs. | toxic dilution of | dilutions | | | |
| cell culture sensitivity to | the neutralized | | | | |
| a virus) | Product | | | | |
| | (~1:1000) | | | | |
| Virus Control #2 | none | Virus 10-fold | PBS | 1 | 8 |
| (virus titration) | | dilutions | | | |
| Virus Control #3 | none | Virus 10-fold | PBS, | 1 | 8 |
| (neutralizer vs. virus) | | dilutions | Neutralizer | | |
| Negative Control | none | none | PBS | 1 | 8 |
| (PBS vs. cell culture) | | | | | |

Neutralization Control: The Neutralization Control determines the efficacy of the Test Product activity suppression and is performed prior to the Virucidal Suspension Test for the virus. A neutralization assay is performed per Sponsor's recommendation based upon the active ingredients of the Test Product. Prior to testing, aliquots of MM, Test Product, and PBS are equilibrated to room temperature overnight. The actual ambient temperature is recorded. MM and PBS are added to a sample of the Test Product (80% v/v concentration of the Test Product). An aliquot of the mixture is then transferred to the appropriate neutralizer and mixed thoroughly. The test virus is then added to the Test Product/MM/PBS/Neutralizer mixture and subsequent virus titration made in MM. Then 3 replicates of the Neutralization Control are performed and each dilution is plated in 8 replicates. In the case of excessive cytotoxicity of the Test Product, Sephadex Gel Filtration is applied.

Cytotoxicity Control: MM and PBS are added to samples of the Test Product (80% v/v concentration of the Test Product). An aliquot of the mixture is transferred to the appropriate neutralizer and mixed thoroughly. Subsequent 10-fold dilutions of the neutralized Test Product are made in MM. The dilutions are plated in 8 replicates. In the case of excessive cytotoxicity of the Test Product, Sephadex Gel Filtration is applied.

The evaluation of the cell sensitivity to the virus and virus infectivity are performed as follows:
1. Virus Control #1 determines whether the Test Product/Neutralizer mixture has significant effect upon the cell culture sensitivity to the virus. The lowest non-toxic dilution of the Product/Neutralizer mixture is plated onto established cell cultures in plate wells designated for the virus titration. After 1 hour of incubation, the Test Product/Neutralizer mixture is removed and virus dilutions are plated. Each dilution is plated in 8 replicates. In the case of excessive cytotoxicity of the Test Product, Sephadex Gel Filtration is applied to the Test Product/Neutralizer mixture.
2. Virus Control #2 determines the sensitivity of cells to the test virus when not treated with the Product/Neutralizer mixture. GM is replaced by PBS in the wells designated for the virus titration. After 1 hour of incubation, the PBS is removed and virus dilutions plated. Each dilution is plated in eight 8 replicates.
3. Virus Control #3 determines the inhibitory effect of the chemical neutralization or Sephadex filtration on the virus infectivity. One of the lower dilutions of the Test Virus is added to the appropriate neutralizer and subsequent dilutions are made in MM. Each dilution is plated in 8 replicates.
4. Negative control. Cell culture monolayers serve as the negative control. The GM is replaced by PBS in all Negative control wells. After 1 hour of incubation, the PBS is replaced by MM with at least 8 replicates.

The plates are incubated in a CO₂ incubator for 5 to 14 days at the appropriate temperature. Cytopathic/cytotoxic effects are monitored daily using an Inverted Compound Microscope.

VIRUCIDAL SUSPENSION TEST: The Virucidal Suspension Test includes the following test conditions and controls:

TABLE 83
Test conditions and controls

| | | Controls | |
|---|---|---|---|
| | | Virus | Cytotoxicity |
| Conditions | Test | Control | Control |
| Concentration of Antiseptic | 80% (v/v) | — | 80% (v/v) |
| Contact Temperature | Ambient | Ambient | Ambient |
| Contact Time | 30 sec., 1 min., | 3 min. | — |
| | 3 min. | | |
| Interfering Substances | PBS | PBS | PBS |
| Number of tests per contact | 1 | 1 | 1 |
| time | | | |
| Number of Replicates per test | 8 | 8 | 8 |

1. Test: Prior to testing, aliquots of MM, Test Product and PBS are equilibrated to room temperature overnight. The actual ambient temperature is recorded. The appropriate amounts of PBS and the Test Virus are added to a sample of the Test Product and mixed thoroughly to achieve the 80% (v/v) concentration of the Test Product. The Test Virus is then exposed to the Test Product for 30 seconds, 1 minute, and 3 minutes, timed using a calibrated minute/second timer. Immediately after each exposure, the Test Virus/Test Product suspension is neutralized and 10-fold dilutions made in MM. Each dilution is plated in 8 replicates. In the case of excessive cytotoxicity of the Test Product, Sephadex Gel Filtration is applied.
2. Virus Control: The appropriate amount of PBS and the Test Virus is added to the MM and exposed for 3 minutes, at room temperature, timed using a calibrated minute/second timer. The mixture is subsequently 10-fold diluted in MM. Each dilution is plated in 8 replicates.
3. Cytotoxicity Control: MM and PBS are added to samples of the Test Product, in simulation of the virus inoculum (80% v/v concentration of the Test Product). The mixture is added to the Neutralizer aliquots and mixed thoroughly. Subsequent 10-fold dilutions of the neutralized Test Product are made in MM. The dilutions are plated in 8 replicates. In the case of excessive cytotoxicity of the Test Product, Sephadex Gel Filtration is applied.
4. Cell culture control: Intact cell culture monolayers serve as a cell culture viability control. The GM is replaced by MM in all Negative control wells (at least 8 replicates).
5. The plates are incubated in a CO₂ incubator for 5 to 14 days at the appropriate temperature. Cytopathic/cytotoxic effect is monitored daily using an Inverted Compound Microscope.

REFERENCE INACTIVATION TEST: The Reference Product Test includes the following parameters:

TABLE 84
Reference product test parameters

| | | Controls | |
|---|---|---|---|
| | Reference | Virus | Cytotoxicity |
| Conditions | Inactivation Test | Control | Control |
| Concentration of | 50% (v/v) | — | 50% (v/v) |
| Antiseptic | | | |
| Contact Temperature | ambient | ambient | ambient |
| Contact Time | 30 min. and 60 min. | 30 min. and | — |
| | | 60 min. | |
| Interfering Substances | PBS | PBS | PBS |
| Number of tests per | 1 | 1 | 1 |
| contact time | | | |
| Number of Replicates | 8 | 8 | 8 |
| per test | | | |

1. Reference Product Test: Formaldehyde 1.4% (v/v) is used as a Reference Product to assess the validity of the test. Prior to testing, aliquots of MM, Reference

Product, and PBS are equilibrated to room temperature. The Test Virus and PBS are mixed with an equal amount of Reference Product to achieve a 50% (v/v) concentration of the Reference Product. Each Test Virus is exposed to the Reference Product for 30 minutes and 60 minutes contact time, timed using a calibrated minute/second timer. Immediately after exposure, the Test Virus/Reference Product suspensions are neutralized and subsequent 10-fold dilutions made in MM. Each dilution is plated in 8 replicates. In any case of excessive cytotoxicity of the Reference Product, Sephadex Gel Filtration is applied.
2. Virus Control: The appropriate amount of PBS and Test Virus are added to the MM and exposed for 30 minutes and 60 minutes at room temperature, timed using a calibrated minute/second timer. The mixture is subsequently 10-fold diluted in MM and each dilution is plated in 8 replicates.
3. Reference Product Cytotoxicity Control: The MM is diluted in PBS in simulation of the virus inoculum and mixed with an equal amount of the Reference Product to achieve 50% (v/v) concentration of the Reference Product. An aliquot of the mixture is transferred to the appropriate neutralizer and mixed thoroughly. Subsequent 10-fold dilutions of the neutralized Reference Product are made in MM. The dilutions are plated in 8 replicates. In the case of excessive cytotoxicity of the Reference Product, Sephadex Gel Filtration is applied.
4. Reference Product Neutralization Control: MM and PBS are added to samples of the Test Product, in simulation of the virus inoculum (50% v/v concentration of the Test Product). The mixture is added to the Neutralizer aliquots and mixed thoroughly. The test virus is added to the Reference Product/MM/PBS/Neutralizer mixture and subsequent virus dilutions made in MM. Each dilution is plated in 8 replicates. In the case of excessive cytotoxicity of the Reference Product, Sephadex Gel Filtration is applied.

The plates are incubated in a CO₂ incubator for 5 to 14 days at the appropriate temperature. Cytopathic/cytotoxic effects are monitored daily using an Inverted Compound Microscope.

CALCULATIONS: Viral and toxicity titers are expressed as −Log₁₀) of the 50% titration end point for infectivity. To calculate the viral titer, a 50% tissue culture infectious dose (TCID₅₀) calculation using the Quantal test (Spearman-Kärber Method) is applied.

Log TCID₅₀=L−d(s−0.5)

where: L=−Log₁₀ of the lowest dilution;
d=difference between dilution steps;
s=sum of proportions of positive wells.

The Log₁₀ of infectivity reduction is calculated as follows:
Log₁₀ Reduction Formula:

Log₁₀ Reduction=(Log₁₀ TCID₅₀ of the Virus Control)−(Log₁₀,TCID₅₀ of the Virucidal Suspension Test)

The virucidal efficacy of the Test Product is presented as a percent of 4 Log₁₀ reduction of viral infectivity (99.99% virucidal efficacy corresponds to 4 Log₁₀, reduction of viral infectivity).

TEST ACCEPTANCE CRITERIA: A valid test requires that:
1. Comparative virus titrations (Virus Control #1, #2 and #3) show differences of <1 Log₁₀;
2. The Test Product is fully neutralized immediately after timed exposure such that the difference between mean value of logarithmic titers of the Neutralization Test and mean value of logarithmic titers of Virus Controls result in ≦0.5 Log₁₀;
3. At least 4 Log₁₀ of TCID₅₀ is recovered from the Virus Control in the Virucidal Suspension Test;
4. Cells in the Negative control wells and Cell Culture Control wells are viable and attached to the bottom of the well;
5. The medium is free of contamination in all wells of the plate; and
6. Cytotoxicity of the Test Product and Reference Product does not affect cell morphology in the dilutions necessary to demonstrate ≧4 Log₁₀, reduction of the virus.

Although the invention has been described with reference to certain specific embodiments, various modifications thereof will be apparent to those skilled in the art without departing from the spirit and scope of the invention. All such modifications as would be apparent to one skilled in the art are intended to be included within the scope of the following claims.

1-43. (canceled)

44. An antiseptic composition comprising a wide spectrum antiseptic component and optionally one or more of an anti-inflammatory agent, an analgesic, or an anaesthetic, said wide spectrum antiseptic component comprising an alcohol, one or more anti-microbial phenol compounds, one or more anti-microbial agents, a solvent, and optionally one or more non-ionic detergents,
with the proviso that when one or more of the anti-inflammatory agent, analgesic, or anaesthetic is absent, the one or more non-ionic detergents are also absent, and when the wide spectrum antiseptic component comprises one or more non-ionic detergents, the antiseptic composition comprises one or more of an anti-inflammatory agent, an analgesic, or an anaesthetic.

45. The antiseptic composition according to claim 44, wherein said antiseptic composition comprises one or more non-ionic detergents and one or more of an anti-inflammatory agent, an analgesic, or an anaesthetic.

46. The antiseptic composition according to claim 45, wherein the one or more of an anti-inflammatory agent, an analgesic, or an anaesthetic is an anti-inflammatory agent.

47. The antiseptic composition according to claim 46, wherein said anti-inflammatory agent is present in an amount of from about 0.01% (w/w) to about 5% (w/w).

48. The antiseptic composition according to claim 46, wherein said anti-inflammatory agent is present in an amount of from about 0.01% (w/w) to about 2% (w/w).

49. The antiseptic composition according to claim 46, wherein said anti-inflammatory agent is a very potent, potent, moderately potent or mild steroid.

50. The antiseptic composition according to claim 46, wherein said anti-inflammatory agent is betamethasone valerate, or hydrocortisone.

51. The antiseptic composition according to claim 44, wherein said wide spectrum antiseptic component comprises from about 50% (w/w) to about 95% (w/w) of the alcohol.

52. The antiseptic composition according to claim 44, wherein said wide spectrum antiseptic component comprises from about 60% (w/w) to about 80% (w/w) of the alcohol.

53. The antiseptic composition according to claim 44, wherein said alcohol is ethanol, methanol, 1-propanol, or combinations thereof.

54. The antiseptic composition according to claim 44, wherein said alcohol is ethanol.

55. The antiseptic composition according to claim 44, wherein said one or more antimicrobial phenol compounds comprise o-phenylphenol.

56. The antiseptic composition according to claim 44, wherein said one or more antimicrobial phenol compounds is o-phenylphenol.

57. The antiseptic composition according to claim 44, wherein said wide spectrum antiseptic component comprises one antimicrobial phenol compound.

58. The antiseptic composition according to claim 55, wherein said one or more anti-microbial phenol compounds are each present in an amount of from about 0.001% (w/w) to about 5% (w/w).

59. The antiseptic composition according to claim 55, wherein said one or more anti-microbial phenol compounds are each present in an amount of from about 0.1% (w/w) to about 0.5% (w/w).

60. The antiseptic composition according to claim 44, wherein said one or more antimicrobial agents comprise benzalkonium chloride.

61. The antiseptic composition according to claim 44, wherein said one or more antimicrobial agents is benzalkonium chloride.

62. The antiseptic composition according to claim 44, wherein said one or more antimicrobial agents are benzalkonium chloride and chlorhexidine gluconate.

63. The antiseptic composition according to claim 44, wherein said wide spectrum antiseptic component comprises one antimicrobial agent.

64. The antiseptic composition according to claim 44, wherein said wide spectrum antiseptic component comprises two antimicrobial agents.

65. The antiseptic composition according to claim 60, wherein said one or more antimicrobial agents are each present in an amount of from about 0.001% (w/w) to about 5% (w/w).

66. The antiseptic composition according to claim 60, wherein said one or more antimicrobial agents are each present in an amount of from about 0.05% (w/w) to about 2% (w/w).

67. The antiseptic composition according to claim 44, wherein said solvent is water.

68. The antiseptic composition according to claim 67, wherein said solvent is deionized, double distilled water.

69. The antiseptic composition according to claim 44, wherein said wide spectrum antiseptic component comprises a non-ionic detergent.

70. The antiseptic composition according to claim 69, wherein said non-ionic detergent is present in an amount of from about 0.2% (w/w) to about 10% (w/w).

71. The antiseptic composition according to claim 69, wherein said non-ionic detergent is present in an amount of from about 0.04% (w/w) to about 0.1% (w/w).

72. The antiseptic composition according to claim 69, wherein said non-ionic detergent is nonoxynol-9, nonoxynol-10 or nonoxynol-11.

73. The antiseptic composition according to claim 69, wherein said non-ionic detergent is nonoxynol-9.

74. The antiseptic composition according to claim 44, wherein said wide spectrum antiseptic component comprises an alcohol, an antimicrobial phenol compound, two antimicrobial agents, a non-ionic detergent, and a solvent.

75. The antiseptic composition according to claim 74, wherein the alcohol is ethanol, the antimicrobial phenol compound is o-phenylphenol, the two antimicrobial agents are benzalkonium chloride and chlorhexidine gluconate, the non-ionic detergent is nonoxynol-9 and the solvent is deionized, double-distilled water.

76. The antiseptic composition according to claim 75, wherein ethanol is present in an amount between about 60% and about 80% (w/w), o-phenylphenol is present in an amount between about 0.1% and about 0.5% (w/w), benzalkonium chloride is present in an amount between about 0.1% and about 2% (w/w), chlorhexidine gluconate is present in an amount between about 0.05% and about 0.5% (w/w), nonoxynol-9 is present in an amount between about 0.04% and about 0.1% (w/w).

77. The antiseptic composition according to claim 44, further comprising one or more formulating agent.

78. The antiseptic composition according to claim 44, wherein said antiseptic composition is formulated as a liquid, semi-liquid, foam or gel.

79. A kit comprising the antiseptic composition according to claim 44, and instructions for use.

80. A method of treating a dermal, fungal, cuticle or genital infection in a subject, comprising administering to said subject an effective amount of an antiseptic composition comprising a wide spectrum antiseptic component, said wide spectrum antiseptic component comprising an alcohol, one or more antimicrobial phenol compounds, one or more antimicrobial agents, a solvent, and optionally one or more non-ionic detergents.

81. The method according to claim 80, wherein said antiseptic composition further comprises one or more of an anti-inflammatory agent, an analgesic, or an anaesthetic.

82. The method according to claim 80, wherein said antiseptic composition further comprises an anti-inflammatory agent.

83. The method according to claim 80, wherein said method is for treating a dermal infection.

84. The method according to claim 80, wherein said dermal infection is caused by a fungus.

85. The method according to claim 84, wherein said fungus is of the genus Trichophyton, Aspergillus, or Candida.

86. The method according to claim 84, wherein said dermal infection is Tinea pedis (athlete's foot), Tinea corporis (ring worm), Tinea versicolor, Tinea cruris (jock itch), Tinea manuum (hand and palm infections).

87. The method according to claim 80, wherein said dermal infection is caused by a bacterium.

88. The method according to claim 87, wherein said bacterium is a Streptococcus, Staphylococcus, Mycobacterium, Escherichia, Enterococcus, Pseudomonas, Proteus, Salmonella, or Propionibacterium.

89. The method according to claim 86, wherein said dermal infection is cellulitis, acne or impetigo.

90. The method according to claim 80, wherein said wide spectrum antiseptic component comprises from about 50% (w/w) to about 95% (w/w) of the alcohol.

91. The method according to claim 80, wherein said wide spectrum antiseptic component comprises from about 60% (w/w) to about 80% (w/w) of the alcohol.

92. The method according to claim 80, wherein said alcohol is ethanol, methanol, 1-propanol, or combinations thereof.

93. The method according to claim 80, wherein said alcohol is ethanol.

94. The method according to claim 80, wherein said one or more antimicrobial phenol compounds comprise o-phenylphenol.

95. The method according to claim 80, wherein said one or more antimicrobial phenol compounds is o-phenylphenol.

96. The method according to claim 80, wherein said wide spectrum antiseptic component comprises one antimicrobial phenol compound.

97. The method according to claim 94, wherein said one or more anti-microbial phenol compounds are each present in a amount of from about 0.001% (w/w) to about 5% (w/w).

98. The method according to claim 94, wherein said one or more anti-microbial phenol compounds are each present in a amount of from about 0.1% (w/w) to about 0.5% (w/w).

99. The method according to claim 80, wherein said one or more antimicrobial agents comprise benzalkonium chloride.

100. The method according to claim 80, wherein said one or more antimicrobial agents is benzalkonium chloride.

101. The method according to claim 80, wherein said one or more antimicrobial agents are benzalkonium chloride and chlorhexidine gluconate.

102. The method according to claim 80, wherein said wide spectrum antiseptic component comprises one antimicrobial agent.

103. The method according to claim 80, wherein said wide spectrum antiseptic component comprises two antimicrobial agents.

104. The method according to claim 99, wherein said one or more antimicrobial agents are each present in an amount of from about 0.001% (w/w) to about 5% (w/w).

105. The method according to claim 99, wherein said one or more antimicrobial agents are each present in an amount of from about 0.005% (w/w) to about 2% (w/w).

106. The method according to claim 80, wherein said solvent is water.

107. The method according to claim 106, wherein said solvent is deionized, double distilled water.

108. The method according to claim 80, wherein said wide spectrum antiseptic component comprises a non-ionic detergent.

109. The method according to claim 108, wherein said non-ionic detergent is present in an amount of from about 0.2% (w/w) to about 10% (w/w).

110. The method according to claim 108, wherein said non-ionic detergent is present in an amount of from about 0.04% (w/w) to about 0.1% (w/w).

111. The method according to claim 108, wherein said non-ionic detergent is nonoxynol-9, nonoxynol-10 or nonoxynol-11.

112. The method according to claim 108, wherein said non-ionic detergent is nonoxynol-9.

113. The method according to claim 82, wherein said anti-inflammatory agent is present in an amount of from about 0.01% (w/w) to about 5% (w/w).

114. The method according to claim 82, wherein said anti-inflammatory agent is present in an amount of from about 0.01% (w/w) to about 2% (w/w).

115. The method according to claim 82, wherein said anti-inflammatory agent is a very potent, potent, moderately potent or mild steroid.

116. The method according to claim 82, wherein said anti-inflammatory agent is betamethasone valerate, or hydrocortisone.

117. The method according to claim 80, wherein said wide spectrum antiseptic component comprises an alcohol, an antimicrobial phenol compound, two antimicrobial agents, a non-ionic detergent, and a solvent.

118. The method according to claim 117, wherein the alcohol is ethanol, the antimicrobial phenol compound is o-phenylphenol, the two antimicrobial agents are benzalkonium chloride and chlorhexidine gluconate, the non-ionic detergent is nonoxynol-9 and the solvent is deionized, double-distilled water.

119. The method according to claim 117, wherein ethanol is present in an amount between about 60% and about 80% (w/w), o-phenylphenol is present in an amount between about 0.1% and about 0.5% (w/w), benzalkonium chloride is present in an amount between about 0.1% and about 2%, chlorhexidine gluconate is present in an amount between about 0.005% to about 0.5% (w/w), nonoxynol-9 is present in an amount between about 0.04% and about 0.1%.

120. The method according to claim 80, wherein said antiseptic composition further comprises one or more formulating agent.

121. The method according to claim 80, wherein said antiseptic composition is formulated as a liquid, semi-liquid, foam or gel.
